(19) United States
(12) Patent Application Publication
(10) Pub. No.: US 2026/0165604 A1
(43) Pub. Date: Jun. 18, 2026

(54) Wearable Band, Strap, or Sleeve with Neuromuscular Activity (e.g. EMG) Sensors

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 19/531,592

(22) Filed: Feb. 5, 2026

Related U.S. Application Data
(63) Continuation-in-part of application No. 19/025,130, filed on Jan. 16, 2025.
(63) Continuation-in-part of application No. 18/369,129, filed on Sep. 15, 2023, now Pat. No. 12,527,521.
Application No. 19/025,130 is a continuation-in-part of application No. 18/900,784, filed on Sep. 29, 2024, now abandoned.
Application No. 18/900,784 is a continuation-in-part of application No. 18/369,129, filed on Sep. 15, 2023, now Pat. No. 12,527,521.
Application No. 18/900,784 is a continuation-in-part of application No. 18/369,129, filed on Sep. 15, 2023, now Pat. No. 12,527,521.
Application No. 18/369,129 is a continuation-in-part of application No. 17/721,866, filed on Apr. 15, 2022, now Pat. No. 11,892,286.
Application No. 17/721,866 is a continuation-in-part of application No. 17/356,377, filed on Jun. 23, 2021, now Pat. No. 11,304,628.
Application No. 17/356,377 is a continuation-in-part of application No. 16/751,245, filed on Jan. 24, 2020, now Pat. No. 11,071,498.
Application No. 16/751,245 is a continuation-in-part of application No. 16/543,056, filed on Aug. 16, 2019, now Pat. No. 10,839,202.
Application No. 16/543,056 is a continuation-in-part of application No. 16/017,439, filed on Jun. 25, 2018, now Pat. No. 10,921,886.
Application No. 16/017,439 is a continuation-in-part of application No. 16/010,448, filed on Jun. 16, 2018, now Pat. No. 10,602,965.
Application No. 16/010,448 is a continuation-in-part of application No. 15/702,081, filed on Sep. 12, 2017, now Pat. No. 10,716,510.
Application No. 15/702,081 is a continuation-in-part of application No. 16/010,448, filed on Jun. 16, 2018, now Pat. No. 10,602,965.
Application No. 16/017,439 is a continuation-in-part of application No. 16/010,448, filed on Jun. 16, 2018, now Pat. No. 10,602,965.
Application No. 16/010,448 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/294,746, filed on Oct. 16, 2016, now Pat. No. 10,627,861.
Application No. 15/294,746 is a continuation-in-part of application No. 14/623,337, filed on Feb. 16, 2015, now Pat. No. 9,582,035.
Application No. 15/294,746 is a continuation-in-part of application No. 14/623,337, filed on Feb. 16, 2015, now Pat. No. 9,582,035.
Application No. 14/623,337 is a continuation-in-part of application No. 15/702,081, filed on Sep. 12, 2017, now Pat. No. 10,716,510.
Application No. 15/702,081 is a continuation-in-part of application No. 15/227,254, filed on Aug. 3, 2016, now Pat. No. 10,321,873.
Application No. 15/227,254 is a continuation-in-part of application No. 15/227,254, filed on Aug. 3, 2016, now Pat. No. 10,321,873.
Application No. 15/227,254 is a continuation-in-part of application No. 14/795,373, filed on Jul. 9, 2015, now abandoned.
Application No. 14/795,373 is a continuation-in-part of application No. 15/130,995, filed on Apr. 17, 2016, now Pat. No. 9,891,718.
Application No. 15/130,995 is a continuation-in-part of application No. 15/079,447, filed on Mar. 24, 2016, now Pat. No. 10,234,934.
Application No. 15/079,447 is a continuation-in-part of application No. 14/736,652, filed on Jun. 11, 2015, now abandoned.
Application No. 14/736,652 is a continuation-in-part of application No. 14/664,832, filed on Mar. 21, 2015, now Pat. No. 9,582,072.
Application No. 14/664,832 is a continuation-in-part of application No. 14/664,832, filed on Mar. 21, 2015, now Pat. No. 9,582,072.
Application No. 14/664,832 is a continuation-in-part of application No. 14/463,741, filed on Aug. 20, 2014, now Pat. No. 9,588,582.
Application No. 14/463,741 is a continuation-in-part of application No. 14/736,652, filed on Jun. 11, 2015, now abandoned.
Application No. 14/736,652 is a continuation-in-part of application No. 14/664,832, filed on Mar. 21, 2015, now Pat. No. 9,582,072.
Application No. 14/664,832 is a continuation-in-part of application No. 14/463,741, filed on Aug. 20, 2014, now Pat. No. 9,588,582.
(60) Provisional application No. 61/878,893, filed on Sep. 17, 2013.
(60) Provisional application No. 61/944,090, filed on Feb. 25, 2014.
(60) Provisional application No. 61/948,124, filed on Mar. 5, 2014.
(60) Provisional application No. 61/976,650, filed on Apr. 8, 2014.
(60) Provisional application No. 62/014,747, filed on Jun. 20, 2014.
(60) Provisional application No. 62/065,032, filed on Oct. 17, 2014.
(60) Provisional application No. 62/086,053, filed on Dec. 1, 2014.
(60) Provisional application No. 62/100,217, filed on Jan. 6, 2015.
(60) Provisional application No. 62/106,856, filed on Jan. 23, 2015.
(60) Provisional application No. 62/111,163, filed on Feb. 3, 2015.
(60) Provisional application No. 62/113,423, filed on Feb. 7, 2015.
(60) Provisional application No. 62/115,691, filed on Feb. 13, 2015.
(60) Provisional application No. 62/150,886, filed on Apr. 22, 2015.
(60) Provisional application No. 62/150,886, filed on Apr. 22, 2015.
(60) Provisional application No. 62/182,473, filed on Jun. 20, 2015.
(60) Provisional application No. 62/187,906, filed on Jul. 2, 2015.
(60) Provisional application No. 62/357,957, filed on Jul. 2, 2016.
(60) Provisional application No. 62/449,735, filed on Jan. 24, 2017.
(60) Provisional application No. 62/538,793, filed on Jul. 30, 2017.
(60) Provisional application No. 62/538,793, filed on Jul. 30, 2017.
(60) Provisional application No. 62/683,237, filed on Jun. 11, 2018.
(60) Provisional application No. 62/683,237, filed on Jun. 11, 2018.
(60) Provisional application No. 62/727,798, filed on Sep. 6, 2018.
(60) Provisional application No. 62/797,266, filed on Jan. 26, 2019.
(60) Provisional application No. 62/797,266, filed on Jan. 26, 2019.

(51) Int. Cl. A61B 5/11; A61B 5/00

(52) U.S. Cl. CPC A61B 5/11; A61B 5/681; A61B 5/6831; A61B 5/742

(57) ABSTRACT

This invention is a band, strap, or sleeve with neuromuscular activity (e.g. EMG) sensors

which is worn on a person's wrist and/or forearm to track the motion of the person's arm, wrist, and fingers and recognize gestures. It can serve as a human-to-computer communication interface. The ventral portion of the device can be wider than the dorsal portion of the device. Also, the device can further comprise: length-adjusting mechanisms at both ends of the band, strap, or sleeve; and tracks or channels on the band, strap, or sleeve along which sensors can slide.

### CROSS-REFERENCE TO RELATED APPLICATIONS

This application is a continuation in part of patent application Ser. No. 19/025,130 filed on 2025-Jan.-16. This application is a continuation in part of patent application Ser. No. 18/369,129 filed on 2023-Sep.-15. patent application Ser. No. 19/025,130 was a continuation in part of patent application Ser. No. 18/900,784 filed on 2024-Sep.-29. patent application Ser. No. 19/025,130 was a continuation in part of patent application Ser. No. 18/369,129 filed on 2023-Aug.-15.

patent application Ser. No. 18/369,129 was a continuation in part of patent application Ser. No. 17/721,866 filed on 2022-Apr.-15 which issued as U.S. Pat. No. 11,892,286 on 2024-Feb.-06. patent application Ser. No. 17/721,866 was a continuation in part of patent application Ser. No. 17/356,377 filed on 2021-Jun.-23 which issued as U.S. Pat. No. 11,304,628 on 2022-Apr.-19. patent application Ser. No. 17/721,866 was a continuation in part of patent application Ser. No. 17/356,377 filed on 2021-Jun.-23 which issued as U.S. Pat. No. 11,304,628 on 2022-Apr.-19. patent application Ser. No. 17/356,377 was a continuation in part of patent application Ser. No. 16/751,245 filed on 2020-Jan.-24 which issued as U.S. Pat. No. 11,071,498 on 2021-Jul.-27.

patent application Ser. No. 16/751,245 was a continuation in part of patent application Ser. No. 16/543,056 filed on 2019-Aug.-16 which issued as U.S. Pat. No. 10,839,202 on 2020-Nov.-17. patent application Ser. No. 16/751,245 claimed the priority benefit of patent provisional application 62/797,266 filed on 2019-Jan.-26. patent application Ser. No. 16/751,245 was a continuation in part of patent application Ser. No. 16/017,439 filed on 2018-Jun.-25 which issued as U.S. Pat. No. 10,921,886 on 2020-Feb.-16. patent application Ser. No. 16/751,245 was a continuation in part of patent application Ser. No. 16/010,448 filed on 2018-Jun.-16 which issued as U.S. Pat. No. 10,602,965 on 2020-Mar.-31. patent application Ser. No. 16/751,245 was a continuation in part of patent application Ser. No. 15/702,081 filed on 2017-Sep.-12 which issued as U.S. Pat. No. 10,716,510 on 2020-Jul.-21.

patent application Ser. No. 16/543,056 claimed the priority benefit of patent provisional application 62/797,266 filed on 2019-Jan.-26. patent application Ser. No. 16/543,056 claimed the priority benefit of patent provisional application 62/727,798 filed on 2018-Sep.-06. patent application Ser. No. 16/543,056 was a continuation in part of patent application Ser. No. 16/010,448 filed on 2018-Jun.-16 which issued as U.S. Pat. No. 10,602,965 on 2020-Mar.-31.

patent application Ser. No. 16/017,439 was a continuation in part of patent application Ser. No. 16/010,448 filed on 2018-Jun.-16 which issued as U.S. Pat. No. 10,602,965 on 2020-Mar.-31. patent application Ser. No. 16/017,439 claimed the priority benefit of patent provisional application 62/683,237 filed on 2018-Jun.-11. patent application Ser. No. 16/017,439 was a continuation in part of patent application Ser. No. 14/795,373 filed on 2015-Jul.-09. patent application Ser. No. 16/017,439 was a continuation in part of patent application Ser. No. 15/725,330 filed on 2017-Oct.-05.

patent application Ser. No. 15/725,330 was a continuation in part of patent application Ser. No. 15/431,769 filed on 2017-Feb.-13. patent application Ser. No. 15/431,769 was a continuation in part of patent application Ser. No. 15/294,746 filed on 2016-Oct.-16. patent application Ser. No. 15/431,769 was a continuation in part of patent application Ser. No. 14/623,337 filed on 2015-Feb.-16. patent application Ser. No. 15/294,746 was a continuation in part of patent application Ser. No. 14/623,337 filed on 2015-Feb.-16.

patent application Ser. No. 14/623,337 claimed the priority benefit of provisional patent application 62/115,691 filed on 2015-Feb.-13. patent application Ser. No. 14/623,337 claimed the priority benefit of provisional patent application 62/113,423 filed on 2015-Feb.-07. patent application Ser. No. 14/623,337 claimed the priority benefit of provisional patent application 62/111,163 filed on 2015-Feb.-03. patent application Ser. No. 14/623,337 claimed the priority benefit of provisional patent application 62/106,856 filed on 2015-Jan.-23. patent application Ser. No. 14/623,337 claimed the priority benefit of provisional patent application 62/100,217 filed on 2015-Jan.-06. patent application Ser. No. 14/623,337 claimed the priority benefit of provisional patent application 61/948,124 filed on 2014-Mar.-05. patent application Ser. No. 14/623,337 claimed the priority benefit of provisional patent application 61/944,090 filed on 2014-Feb.-25.

patent application Ser. No. 16/010,448 claimed the priority benefit of patent provisional application 62/683,237 filed on 2018-Jun.-11. patent application Ser. No. 16/010,448 was a continuation in part of patent application Ser. No. 15/702,081 filed on 2017-Sep.-12 which issued as U.S. Pat. No. 10,716,510 on 2020-Jul.-21. patent application Ser. No. 16/010,448 claimed the priority benefit of patent provisional application 62/538,793 filed on 2017-Jul.-30. patent application Ser. No. 16/010,448 was a continuation in part of patent application Ser. No. 15/227,254 filed on 2016-Aug.-03 which issued as U.S. Pat. No. 10,321,873 on 2019-Jun.-18.

patent application Ser. No. 15/702,081 claimed the priority benefit of patent provisional application 62/538,793 filed on 2017-Jul.-30. patent application Ser. No. 15/702,081 claimed the priority benefit of patent provisional application 62/449,735 filed on 2017-Jan.-24. patent application Ser. No. 15/702,081 was a continuation in part of patent application Ser. No. 15/227,254 filed on 2016-Aug.-03 which issued as U.S. Pat. No. 10,321,873 on 2019-Jun.-18. patent application Ser. No. 15/702,081 was a continuation in part of patent application Ser. No. 14/795,373 filed on 2015-Jul.-09.

patent application Ser. No. 15/227,254 claimed the priority benefit of patent provisional application 62/357,957 filed on 2016-Jul.-02. patent application Ser. No. 15/227,254 was a continuation in part of patent application Ser. No. 15/130,995 filed on 2016-Apr.-17 which issued as U.S. Pat. No. 9,891,718 on 2018-Feb.-13. patent application Ser. No. 15/227,254 was a continuation in part of patent application Ser. No. 15/079,447 filed on 2016-Mar.-24 which issued as U.S. Pat. No. 10,234,934 on 2019-Mar.-19. patent application Ser. No. 15/227,254 was a continuation in part of patent application Ser. No. 14/736,652 filed on 2015-Jun.-11. patent application Ser. No. 15/227,254 was a continuation in part of patent application Ser. No. 14/664,832 filed on 2015-Mar.-21 which issued as U.S. Pat. No. 9,582,072 on 2017-Feb.-28.

patent application Ser. No. 15/130,995 claimed the priority benefit of patent provisional application 62/150,886 filed on 2015-Apr.-22. patent application Ser. No. 15/079,447 claimed the priority benefit of patent provisional application 62/150,886 filed on 2015-Apr.-22. patent application Ser. No. 15/079,447 was a continuation in part of patent application Ser. No. 14/664,832 filed on 2015-Mar.-21 which issued as U.S. Pat. No. 9,582,072 on 2017-Feb.-28. patent application Ser. No. 15/079,447 was a continuation in part of patent application Ser. No. 14/463,741 filed on 2014-Aug.-20 which issued as U.S. Pat. No. 9,588,582 on 2017-Mar.-07.

patent application Ser. No. 14/795,373 claimed the priority benefit of patent provisional application 62/187,906 filed on 2015-Jul.-02. patent application Ser. No. 14/795,373 claimed the priority benefit of patent provisional application 62/182,473 filed on 2015-Jun.-20. patent application Ser. No. 14/795,373 was a continuation in part of patent application Ser. No. 14/736,652 filed on 2015-Jun.-11. patent application Ser. No. 14/795,373 claimed the priority benefit of patent provisional application 62/086,053 filed on 2014-Dec.-01. patent application Ser. No. 14/795,373 claimed the priority benefit of patent provisional application 62/065,032 filed on 2014-Oct.-17.

patent application Ser. No. 14/736,652 was a continuation in part of patent application Ser. No. 14/664,832 filed on 2015-Mar.-21 which issued as U.S. Pat. No. 9,582,072 on 2017-Feb.-28. patent application Ser. No. 14/736,652 claimed the priority benefit of patent provisional application 62/014,747 filed on 2014-Jun.-20. patent application Ser. No. 14/664,832 was a continuation in part of patent application Ser. No. 14/463,741 filed on 2014-Aug.-20 which issued as U.S. Pat. No. 9,588,582 on 2017-Mar.-07. patent application Ser. No. 14/664,832 claimed the priority benefit of patent provisional application 61/976,650 filed on 2014-Apr.-08. patent application Ser. No. 14/463,741 claimed the priority benefit of patent provisional application 61/878,893 filed on 2013-Sep.-17.

The entire contents of these applications are incorporated herein by reference.

FEDERALLY SPONSORED RESEARCH: Not Applicable

SEQUENCE LISTING OR PROGRAM: Not Applicable

BACKGROUND—FIELD OF INVENTION: This invention relates to wearable devices and systems for measuring body motion and/or muscle activity.

### INTRODUCTION

A band, strap, or sleeve with neuromuscular activity (e.g. electromyography) sensors which is worn on a person's wrist and/or forearm can track the motion of a person's arm, wrist, and fingers in order to recognize gestures and serve as a human-to-computer communication interface. A band, strap, or sleeve without its own display screen can function as a watch band which can hold different types and brands of smart watch housings. A band, strap, or sleeve with a built-in display screen can function as a complete wearable computing and biosensing device.

### REVIEW OF THE RELEVANT ART

U.S. patent application 20090327171 (Tan et al., Dec. 31, 2009, “Recognizing Gestures from Forearm EMG Signals”) discloses a machine learning model which recognizes gestures based on data from EMG sensors. U.S. Pat. No. 8,170,656 (Tan et al., May 1, 2012, “Wearable Electromyography-Based Controllers for Human-Computer Interface”) and U.S. Pat. No. 9,037,530 (Tan et al., May 19, 2015, “Wearable Electromyography-Based Human-Computer Interface”) disclose a wearable EMG-based controller which provides a human-computer interface (HCl) for interacting with devices via electrical signals generated by movement of a person's muscles.

U.S. Pat. No. 8,704,757 (Kurashima et al., Apr. 22, 2014, “Input System and Input Apparatus”) discloses myoelectric sensors on an area between a wrist of the person and bases of a second finger to a fifth finger to detect myoelectric signals depending on hand motion. U.S. patent application 20140198034 (Bailey et al., Jul. 17, 2014, “Muscle Interface Device and Method for Interacting with Content Displayed on Wearable Head Mounted Displays”) discloses a muscle interface device and method for interacting with content displayed on a wearable head mounted display.

U.S. patent application 20140198035 (Bailey et al., Jul. 17, 2014, “Wearable Muscle Interface Systems, Devices and Methods That Interact with Content Displayed on an Electronic Display”) and U.S. Pat. No. 10,528,135 (Bailey et al., Jan. 7, 2020, “Wearable Muscle Interface Systems, Devices and Methods That Interact with Content Displayed on an Electronic Display”) disclose a wearable muscle interface device and a wearable head-mounted display, as well as methods to effect interactions between the user and content displayed on the wearable head-mounted display. U.S. patent application 20140240223 (Lake et al., Aug. 28, 2014, “Method and Apparatus for Analyzing Capacitive EMG and IMU Sensor Signals for Gesture Control”) discloses a muscle interface device comprising a sensor worn on a forearm to recognize a gestures made by a user for interacting with a controllable connected device.

U.S. patent application 20140240103 (Lake et al., Aug. 28, 2014, “Methods and Devices for Combining Muscle Activity Sensor Signals and Inertial Sensor Signals for Gesture-Based Control”) discloses a wearable electronic device including a band worn on the forearm with at least one muscle activity sensor, at least one inertial sensor, and a processor. U.S. patent application 20140249397 (Lake et al., Sep. 4, 2014, “Differential Non-Contact Biopotential Sensor”) discloses a differential non-contact sensor system for measuring biopotential signals.

U.S. Pat. No. 8,892,479 (Tan et al., Nov. 18, 2014, “Recognizing Finger Gestures from Forearm EMG Signals”) discloses a machine learning model which recognizes gestures from EMG sensors. U.S. patent application 20150025355 (Bailey et al., Jan. 22, 2015, “Systems, Articles and Methods for Strain Mitigation in Wearable Electronic Devices”) discloses wearable electronic devices which provide adaptive physical coupling between electrically coupled components. U.S. patent application 20150070270 (Bailey et al., Mar. 12, 2015, “Systems, Articles, and Methods for Electromyography-Based Human-Electronics Interfaces”) discloses a human-electronics interface in which at least two wearable electromyography (EMG) devices are operated to control an electronic device.

U.S. patent application 20150084860 (Aleem et al., Mar. 26, 2015, “Systems, Articles, and Methods for Gesture Identification in Wearable Electromyography Devices”) discloses systems, articles, and methods for gesture identification which do not require an extensive training procedure. U.S. patent application 20150109202 (Ataee et al., Apr. 23, 2015, “Systems, Articles, and Methods for Gesture Identification in Wearable Electromyography Devices”) discloses a wearable electromyography (EMG) device with multiple EMG sensors and a processor for gesture identification. U.S. patent application 20150124566 (Lake et al., May 7, 2015, “Systems, Articles and Methods for Wearable Electronic Devices Employing Contact Sensors”) discloses a wearable electronic device with one or more contact sensors (e.g., capacitive sensors and/or biometric sensors).

U.S. Pat. No. 9,278,453 (Assad, Mar. 8, 2016, “Biosleeve Human-Machine Interface”) discloses systems and methods for sensing human muscle action and gestures in order to control devices. U.S. patent application 20160195928 (Wagner et al., Jul. 7, 2016, “Closed Loop Feedback Interface for Wearable Devices”) and U.S. Pat. No. 9,612,661 (Wagner et al., Apr. 4, 2017, “Closed Loop Feedback Interface for Wearable Devices”) disclose a gesture controlled system wearable by a user and operationally connected to a computerized device. U.S. patent application 20160313801 (Wagner et al., Oct. 27, 2016, “Method and Apparatus for a Gesture Controlled Interface for Wearable Devices”) and U.S. Pat. No. 9,720,515 (Wagner et al., Aug. 1, 2017, “Method and Apparatus for a Gesture Controlled Interface for Wearable Devices”) discloses a gesture-controlled interface apparatus with biopotential sensors and a processor.

U.S. Pat. No. 9,872,650 (Vice, Jan. 23, 2018, “Electrodermal Interface System”) discloses a communication system, method, and computer program product supported by electro-dermal monitoring physiological signals at the surface of the skin. U.S. Pat. No. 10,070,799 (Ang et al., Sep. 11, 2018, “Detecting and Using Body Tissue Electrical Signals”) discloses measurement of bio-potentials on a person's skin to track body motions. U.S. Pat. No. 10,095,317 (Lee et al., Oct. 9, 2018, “System for Hand Gesture Detection”) discloses a system for hand gesture detection using a wrist-worn device with skin electrodes.

U.S. Pat. No. 10,152,082 (Bailey, Dec. 11, 2018, “Systems, Articles and Methods for Wearable Electronic Devices That Accommodate Different User Forms”) discloses a wearable electronic device with a set of pod structures arranged in an annular configuration having a variable circumference, with adaptive physical coupling between adjacent pairs of pod structures.

U.S. patent application 20190212817 (Kaifosh et al., Jul. 11, 2019, “Methods and Apparatus for Predicting Musculo-Skeletal Position Information Using Wearable Autonomous Sensors”) discloses a method and apparatus for providing a dynamically-updated computerized musculo-skeletal representation comprising a plurality of rigid body segments connected by joints. U.S. patent application 20190228330 (Kaifosh et al., Jul. 25, 2019, “Handstate Reconstruction Based on Multiple Inputs”) discloses methods and systems for dynamically reconstructing handstate information based on multiple inputs.

U.S. patent application 20230270363 (Qazi et al., Aug. 31, 2023, “Smart Electrodes for Sensing Signals and Processing Signals Using Internally-Housed Signal-Processing Components at Wearable Devices and Wearable Devices Incorporating the Smart Electrodes”) discloses smart electrodes with a conductive exterior surface which contacts a person's skin to receive neuromuscular signals.

### SUMMARY OF THE INVENTION

This invention is a band, strap, or sleeve with neuromuscular activity (e.g. electromyography) sensors which is worn on a person's wrist and/or forearm. It can be used to track the motion of a person's arm, wrist, and fingers in order to recognize gestures and serve as a human-to-computer communication interface. In an example, it can function as a watch band which can hold different types and brands of smart watch housings. In an example, it can further comprise its own display screen and function as a smart watch on its own or as a more-general wearable computing and biosensing device.

In an example, the portion of the device which spans the ventral surface of a person's wrist and/or forearm can be wider than the portion of the device which spans the dorsal surface of the person's wrist and/or forearm. In an example, the device can further comprise length-adjusting mechanisms at both ends of the band, strap, or sleeve (e.g. where the band connects to a display screen or watch housing). In an example, there can be channels or tracks on the band, strap, or sleeve along which sensors can be moved to more accurately record the activity of specific muscles and/or customize sensor configuration to the anatomy of a specific person.

### BRIEF INTRODUCTION TO THE FIGURES

FIG. 1 shows a band with neuromuscular activity sensors wherein the band is wider than a display screen.

FIG. 2 shows a band with neuromuscular activity sensors wherein band ends taper toward a display screen.

FIG. 3 shows a band with neuromuscular activity sensors wherein the band bifurcates on a dorsal side.

FIG. 4 shows a band with neuromuscular activity sensors wherein the band bifurcates at an acute angle.

FIG. 5 shows a band with neuromuscular activity sensors wherein the band bifurcates on a lateral side.

FIG. 6 shows a band with neuromuscular activity sensors wherein bifurcated branches of the band are connected on a ventral side.

FIG. 7 shows a band with neuromuscular activity sensors wherein the band has a circumferential array of holes.

FIG. 8 shows a band with neuromuscular activity sensors wherein the band has two circumferential arrays of holes.

FIG. 9 shows a band with neuromuscular activity sensors wherein the band has a circumferential array of longitudinal holes.

FIG. 10 shows a laterally-symmetric undulating band with neuromuscular activity sensors.

FIG. 11 shows a dual band with neuromuscular activity sensors.

FIG. 12 shows a dual band with neuromuscular activity sensors wherein a display screen is between proximal and distal bands.

FIG. 13 shows a dual band with neuromuscular activity sensors wherein a display screen is on a distal band.

FIG. 14 shows a band with neuromuscular activity sensors wherein proximal and distal sides of the band are asymmetric.

FIG. 15 shows a band with neuromuscular activity sensors with a distal protrusion which holds a display screen.

FIG. 16 shows a laterally-asymmetric undulating band with neuromuscular activity sensors.

FIG. 17 shows a band with neuromuscular activity sensors wherein there are elastic sections at the ends of the band.

FIG. 18 shows a band with neuromuscular activity sensors wherein there are telescoping sections at the ends of the band.

FIG. 19 shows a band with neuromuscular activity sensors wherein there are overlapping sections at the ends of the band.

FIG. 20 shows a band with neuromuscular activity sensors wherein there are coil housings at the ends of the band.

FIG. 21 shows a fabric band or sleeve with neuromuscular activity sensors formed on the fabric.

FIG. 22 shows a three-ring band with neuromuscular activity sensors.

FIG. 23 shows a fabric band or sleeve with neuromuscular activity sensors attached to the fabric.

FIG. 24 shows a shirt sleeve or cuff with neuromuscular activity sensors.

FIG. 25 shows a band with diagonal neuromuscular activity sensors.

FIG. 26 shows a band with offset circumferential rings of neuromuscular activity sensors.

FIGS. 27 and 28 show a band wherein neuromuscular activity sensors slide along a circumferential track.

FIGS. 29 and 30 show a band with modular neuromuscular activity sensors.

FIG. 31 shows a band with expandable chambers which change the amounts by which neuromuscular activity sensors protrude out from the band.

FIG. 32 shows a band with pistons and/or solenoids which change the amounts by which neuromuscular activity sensors protrude out from the band.

FIG. 33 shows a band wherein the amounts by which neuromuscular activity sensors protrude out from the band are changed by rotation.

FIG. 34 shows a band with fluid-filled chambers in neuromuscular activity sensors.

FIG. 35 shows a longitudinally-flared dual band with neuromuscular activity sensors and an arcuate display screen.

FIG. 36 shows a dual band with neuromuscular activity sensors and a display screen.

FIG. 37 shows a longitudinally-flared dual band with neuromuscular activity sensors and a rounded-rectangle display screen.

FIG. 38 shows a bifurcated band with neuromuscular activity sensors and a hexagonal display screen.

FIG. 39 shows a flared bifurcated band with neuromuscular activity sensors and an arcuate display screen.

FIG. 40 shows a band with holes, neuromuscular activity sensors, and an arcuate display screen.

FIG. 41 shows a band with neuromuscular activity sensors wherein the band is an annular sequence of rigid housings and stretchable sections.

FIG. 42 shows a band with neuromuscular activity sensors wherein the band is an annular sequence of rigid housings and telescoping sections.

FIG. 43 shows a fabric band or sleeve with circumferential arrays of neuromuscular activity sensors.

FIG. 44 shows a fabric band or sleeve with neuromuscular activity sensors on ventral and lateral portions.

FIG. 45 shows a band or sleeve with EMG sensors and bending-based motion sensors.

FIG. 46 shows a band or sleeve with EMG sensors, bending-based motion sensors, and a display screen.

FIGS. 47 and 48 show bands with laterally-rotatable neuromuscular activity sensors.

FIG. 49 shows a band with laterally-slidable neuromuscular activity sensors.

FIG. 50 shows a telescoping dual band with neuromuscular activity sensors.

### DETAILED DESCRIPTION OF THE FIGURES

In an example, a wearable device with neuromuscular activity sensors can comprise: a band which is configured to be worn around at least 50% of a circumference of a person's wrist or forearm, wherein the average width of a portion of the band on the ventral surface of the person's wrist or forearm is at least 20% greater than the average width of a portion of the band on the dorsal surface of the person's wrist or forearm; and a plurality of neuromuscular activity sensors on the band. In an example, a device can further comprise a display screen or watch housing. In an example, a width of the band can taper toward the display screen or watch housing. In an example, a band can bifurcate into proximal and distal branches.

In an example, a wearable device with neuromuscular activity sensors can comprise: a band which is configured to be worn around at least 50% of a circumference of a person's wrist or forearm; a length-adjusting mechanism on an end of the band; and a plurality of neuromuscular activity sensors on the band. In an example, a device can further comprise a display screen or watch housing. In an example, there can be a length-adjusting mechanism on each end of the band where the band is connected to the display screen or watch housing. In an example, a length-adjusting mechanism can comprise a roller or axle around which an end of the band loops to overlap a portion of the rest of the band. In an example, a length-adjusting mechanism can be an elastic and/or stretchable section. In an example, a length-adjusting mechanism can be a telescoping section. In an example, a length-adjusting mechanism can be a housing in which an end of the band is coiled.

In an example, a wearable device with neuromuscular activity sensors can comprise: a band which is configured to be worn around at least 50% of a circumference of a person's wrist or forearm; a channel or track on the band; and a plurality of neuromuscular activity sensors on the band, wherein one or more of the neuromuscular activity sensors can be selectively moved along the channel or track. In an example, a device can further comprise a display screen or watch housing. In an example, a channel or track can be around a portion of the circumference of the band. In an example, a channel, track, or ring can be centered within a width of the band. In an example, a channel or track can be across a portion of the width of the band. In an example, sensors can be manually moved along the channel or track. In an example, sensors can be automatically moved along the channel or track by an actuator. In an example, sensors can be moved to more accurately record the activity of specific muscles. In an example, sensors can be moved to customize the sensor configuration to the anatomy of a specific person.

In an example, a band, strap, or sleeve with neuromuscular activity (e.g. EMG) sensors and an integrated display screen can function as a human-to-computer interface for (e.g. in combination with) augmented reality eyewear. In an example, a band, strap, or sleeve with neuromuscular activity (e.g. EMG) sensors can be used for motion capture. In an example, a band, strap, or sleeve with neuromuscular activity (e.g. EMG) sensors can record data which is used to measure, monitor, record, and/or recognize wrist, hand, and/or finger motions. In another example, data from EMG sensors on a band can be analyzed to recognize selected hand gestures, wherein these hand gestures control the operation of cursor on a computer screen. In an example, data from neuromuscular activity (e.g. EMG) sensors in a band can be used for medical monitoring and diagnosis, muscle rehabilitation, exercise and training, prosthetic control, and controlling functions of electronic devices.

In an example, a band, strap, or sleeve with neuromuscular activity (e.g. EMG) sensors can be a separate device from a smart watch housing, to which different types and brands of smart watch housings can be removably-attached. In an example, a band, strap, or sleeve with neuromuscular activity (e.g. EMG) sensors can be a watch strap which spans between 70% and 90% of the circumference of a person's wrist. In another example, a band, strap, or sleeve with neuromuscular activity (e.g. EMG) sensors can function as a watch strap which holds a watch housing on a person's wrist by an attachment mechanism selected from the group consisting of: clasp, clip, friction connector, hinge, hook, hook and loop material, loop, magnet, pin, piston, plug, rotating pin, snap, spring, and telescoping component. In an example, a watch band, strap, or sleeve with neuromuscular activity (e.g. EMG) sensors can be used to hold different types and/or brands of watch housings on a person's wrist (and/or forearm).

In another example, a device including a band (or sleeve), EMG sensors, and display screen can collectively comprise a smart watch. In one embodiment, a band, strap, or sleeve with neuromuscular activity (e.g. EMG) sensors can comprise an arm band. In an example, EMG sensors can be incorporated into a cuff or sleeve of a shirt. In an example, ends of a band can be connected by a snap, buckle, or hook. In an example, the ends of a band, strap, or sleeve with neuromuscular activity (e.g. EMG) sensors can be attached to each other by a hook-and-loop fastener. In another example, a watch band with neuromuscular activity (e.g. EMG) sensors can have an adjustable length (e.g. partial circumference), wherein the length of the watch band is adjusted by changing the degree of overlap on ends of the band where the band connected to the watch housing.

In an example, a band or strap with neuromuscular activity (e.g. EMG) sensors can have one or more housings attached to a display screen (and/or watch housing) in which one or more ends of the band are coiled, wherein the length of the band around a person's wrist (and/or forearm) can be adjusted by changing the amounts of the one or more ends which are coiled within the housing. In another example, an end of a band can be adjustably-coiled within a coil housing, thereby changing the length of the rest of the band which encircles (a portion of) the circumference of the person's wrist (and/or forearm). In an example, an end of band can be adjustably-coiled within a housing by an actuator which automatically rotates an axle (or roller) around which the end is coiled.

In another example, the ends of a band (or strap or sleeve) with neuromuscular activity (e.g. EMG) sensors can be connected to a watch housing, wherein the ends of the band roll around cylindrical components where they connect to the watch housing so as to overlap portions of the rest of the band, and wherein the fit (e.g. tightness or looseness) of the band around the person's wrist can be adjusted by adjusting (e.g. changing) the amount by which the ends overlap the rest of the band. In an example, the length of a band around a person's wrist (and/or forearm) can be adjusted by coiling (or uncoiling) one or both ends of the band within one or two housings.

In one embodiment, a band with neuromuscular activity (e.g. EMG) sensors can comprise two stretchable (e.g. elastic) sections and a non-stretchable section, wherein the stretchable sections are closer to (e.g. adjacent to) a display screen (and/or watch housing) than the non-stretchable section. In an example, a band, strap, or sleeve with neuromuscular activity (e.g. EMG) sensors can have a plurality of electronics housings around at least half of its circumference, wherein these housings are interconnected by elastic and/or stretchable material. In an example, a shirt with neuromuscular activity (e.g. EMG) sensors can comprise: a first portion with a first level of elasticity, closeness-of-fit, and/or stretchability; and a second portion with a second level of elasticity, closeness-of-fit, and/or stretchability, wherein the second level is less than the first level, and wherein EMG sensors are on the second portion. In another example, the portion of a band, strap, or sleeve with neuromuscular activity (e.g. EMG) sensors which spans the dorsal surface of a person's wrist or forearm can be more elastic than the portion of the band which spans the ventral surface of the person's wrist or forearm.

In an example, a band, strap, or sleeve with neuromuscular activity (e.g. EMG) sensors can have a variable circumference. In another example, one or more actuators can automatically adjust the fit of a cuff or sleeve with neuromuscular activity (e.g. EMG) sensors on a person's wrist and/or forearm. In an example, the diameter of a band can be automatically adjusted based on analysis of data from EMG sensors. In an example, when signals from one or more EMG sensors on a band are weak or inaccurate, then the fit (e.g. circumference) of the band can be automatically adjusted by one or more electromagnetic actuators.

In an example, a first portion of a band which spans the ventral surface of a person's wrist or forearm and be wider than a second portion of the band which spans the dorsal surface of the person's wrist or forearm. In one embodiment, a maximum (proximal-to-distal) width of a band, strap, or sleeve can be at least 50% greater than a maximum (proximal-to-distal) width of a display screen (and/or watch housing) which is attached to it. In another example, maximum (proximal-to-distal) width of a band, strap, or sleeve can be at least 20% greater than a maximum (proximal-to-distal) width of a smart watch housing. In an example, the average width of a ventral portion of a band can be at least 20% greater than the average width of a dorsal portion of the band. In another example, a band or strap with neuromuscular activity (e.g. EMG) sensors can have between 2 and 6 (sinusoidal) undulations. In an example, the proximal and distal edges of a band, strap, or sleeve can have (sinusoidal) undulations as they curve around (a portion of) the circumference of a person's wrist (and/or forearm). In an example, EMG sensors can be located on convex segments of a band.

In an example, a band with neuromuscular activity (e.g. EMG) sensors can bifurcate into two branches on the lateral surfaces (e.g. between dorsal and ventral) of a person's wrist (and/or forearm). In another example, a dual band or strap with neuromuscular activity (e.g. EMG) sensors can comprise two undulating (e.g. sinusoidal) bands with different phases and/or amplitudes. In an example, a dual band with neuromuscular activity (e.g. EMG) sensors can comprise a proximal band and a distal, wherein the proximal band is closer to the person's wrist (and/or forearm) than the distal band, and wherein a one or both of the proximal and distal bands can be rotated relative to the other band. In another example, a proximal band can be reversibly telescoped into (or out from) a distal band.

In an example, a band or strap with neuromuscular activity (e.g. EMG) sensors can comprise an alternating series of electronic housings (wherein each housing contains one or more EMG sensors) and elastic (e.g. stretchable fabric) sections, wherein the series alternates (back and forth) between electronic housings and elastic sections along the band or strap. In another example, a band with neuromuscular activity (e.g. EMG) sensors can comprise an alternating sequence of rigid (e.g. polymer or metal) housings and stretchable (e.g. fabric) sections around a person's wrist (and/or forearm), wherein the rigid sections are at least 20% wider than the stretchable sections. In an example, a band with neuromuscular activity (e.g. EMG) sensors can comprise an alternating sequence of rigid (e.g. polymer or metal) housings and stretchable (e.g. fabric) sections around a person's wrist (and/or forearm), wherein the stretchable sections collectively span at least 50% more of the circumference of the wrist (and/or forearm) than the rigid sections.

In one embodiment, a band, strap, or sleeve with neuromuscular activity (e.g. EMG) sensors can comprise a plurality of flexibly-connected rigid components. In an example, a band, strap, or sleeve with neuromuscular activity (e.g. EMG) sensors can comprise an alternating series of flexible sections and rigid sections, wherein the EMG sensors are on the rigid sections. In an example, a plurality of rigid components housing neuromuscular activity (e.g. EMG) sensors which is around the perimeter of a band can be connected by a plurality of elastic strips and/or straps. In an example, a band, strap, or sleeve with neuromuscular activity (e.g. EMG) sensors can comprise a ring of fabric between two metal bands around a person's wrist and/or forearm. In another example, an EMG sensor can be made with electroconductive fabric.

In an example, a band, strap, or sleeve with neuromuscular activity (e.g. EMG) sensors can comprise a plurality of rigid sections around a portion of the circumference of a person's wrist and/or forearm; wherein the rigid sections are flexibly-connected by stretchable, elastic, and/or bendable sections; and wherein the stretchable, elastic, and/or bendable sections further comprise flexible (e.g. undulating) electroconductive pathways which transmit power and/or electronic data between the rigid components. In another example, a band, strap, or sleeve with neuromuscular activity (e.g. EMG) sensors which serves as a watch strap can be in wireless electromagnetic communication with electronic components in a watch housing.

In an example, sensors in a band can be in wireless communication with electronics in a watch housing. In another example, there can be a direct electromagnetic connection (e.g. plug, port, and/or outlet) between a band, strap, or sleeve with neuromuscular activity (e.g. EMG) sensors which serves as a watch strap and a watch housing which is held on a wrist by the band, wherein this connection transmits electrical power (e.g. from a battery) and/or transmits data (e.g. data from EMG sensors). In an example, an EMG sensor can be a surface electromyography sensor. In one embodiment, neuromuscular activity sensors can be capacitive EMG sensors. In an example, neuromuscular activity sensors can be surface EMG sensors. In an example, an EMG sensor can be a soft electrode. In an example, neuromuscular activity sensors can comprise pairs of electrodes.

In another example, an EMG sensor can comprise a first layer of material with a first level of electroconductivity and a second layer of material with a second level of electroconductivity, wherein the first layer is closer to the surface of the person's body than the second layer, and wherein the second level is less than the first level. In an example, an EMG sensor can comprise a first layer of material with a first level of permittivity and a second layer of material with a second level of permittivity, wherein the first layer is closer to the surface of the person's body than the second layer, and wherein the second level is less than the first level. In another example, an EMG sensor can comprise an electroconductive layer which is made from an elastomeric polymer which has been doped, impregnated, and/or coated with carbon black. In an example, an EMG sensor can comprise an electroconductive layer which is made from an elastomeric polymer which has been doped, impregnated, and/or coated with silver particles. In an example, an EMG sensor can include a non-conductive substrate.

In an example, an EMG sensor can comprise a dielectric layer and two electroconductive layers. In an example, an EMG sensor can comprise a first layer of dielectric material which is closer to the person's body and a second layer of electroconductive material which is farther from the person's body, wherein the first layer and the second layer are parallel to each other. In another example, an EMG sensor can comprise an electroconductive layer (e.g. plate) which is coated with dielectric material. In one embodiment, an EMG sensor can have a layer of dielectric material. In another example, EMG sensors on a band can be soft, compressible, and/or compliant. In an example, EMG sensors on a band can have one or more Shore OO values between 40 and 80.

In another example, a band, strap, or sleeve with neuromuscular activity (e.g. EMG) sensors can have EMG sensors with hemispherical and/or dome shapes which protrude out from the inner circumference of the band toward the surface of a person's wrist and/or forearm. In an example, an EMG sensor on a band can have a half-cylindrical shape. In an example, an EMG sensor on a band can have a rounded-rectangular shape. In an example, an EMG sensor on a band can have an elliptical or oval shape. In an example, neuromuscular activity sensors can have circular cross-sectional shapes.

In an example, a band or strap with neuromuscular activity (e.g. EMG) sensors can comprise a pleated and/or articulated band and a non-pleated, non-articulated band. In another example, EMG sensors around the circumference of a band can have different sizes, wherein EMG sensors on a portion of the band which spans the ventral surface of a person's wrist are larger than EMG sensors on the rest of the band. In an example, an EMG sensor can be made by printing elastomeric conductive material (e.g. electroconductive ink) onto a low-conductivity textile or fabric. In another example, neuromuscular activity (e.g. EMG) sensors can be attached to a sleeve by printing. In one embodiment, an EMG sensor can comprise a portion of fabric (e.g. a textile) onto which electroconductive yarns, threads, or filaments have been embroidered. In another example, neuromuscular activity (e.g. EMG) sensors can be formed by sewing, braiding, or embroidering electroconductive yarns, threads, or fibers onto a sleeve after the sleeve is formed.

In an example, a band, strap, or sleeve with neuromuscular activity (e.g. EMG) sensors can comprise a first set of EMG sensors distributed around a first circumferential ring on the body-facing surface of the band and a second set of EMG sensors distributed around a second circumferential ring on the body-facing surface of the band, wherein the first and second rings are parallel to each other. In an example, EMG sensors can be on the inner circumference of a band. In an example, neuromuscular activity (e.g. EMG) sensors on a band can be aligned along a single (e.g. proximally-to-distal central) circumferential ring around the band. In an example, neuromuscular activity (e.g. EMG) sensors on a band can be configured in a ring-and-row array, wherein a ring is around the circumference of a person's wrist (and/or forearm) and a row is orthogonal to the ring.

In an example, a band, strap, or sleeve with neuromuscular activity (e.g. EMG) sensors can span the entire circumference of a person's wrist or forearm. In an example, EMG sensors on a band can collectively span between 50% and 80% of the circumference of a wrist or forearm. In an example, at least 50% of EMG sensors on band can be configured to contact the ventral side of a wrist or forearm. In another example, EMG sensors on a portion of a band which spans the dorsal surface of a person's wrist or forearm can farther apart than EMG sensors on a portion of the band which spans the ventral surface of the person's wrist or forearm. In an example, EMG sensors on a portion of a band which spans the ventral surface of a person's wrist can protrude out farther from the band than EMG sensors on the rest of the band. In another example, EMG sensors on a ventral-facing side of a band can be closer together than EMG sensor on the lateral side of the band. In one embodiment, the majority of EMG sensors on band can be configured to contact the ventral side of a wrist or forearm.

In another example, pair-wise-adjacent EMG sensors around the circumference of a band can be separated by different distances. In an example, a neuromuscular activity (e.g. EMG) sensor can have a first configuration in which its longitudinal axis is orthogonal to a circumference of a band and a second configuration in which its longitudinal axis intersects the circumference at an acute angle. In an example, a band, strap, or sleeve with modular neuromuscular activity (e.g. EMG) sensors can have a track, channel, or ring around its inner perimeter, wherein one or more modular sensors can be removably attached to the band, strap, or sleeve on the track, channel, or ring.

In an example, a shirt with modular neuromuscular activity (e.g. EMG) sensors can include a pocket, channel, or pouch into which one or more sensors can be removably inserted. In an example, one or more EMG sensors can be attached (permanently or temporarily) to a shirt, sleeve, and/or cuff by a mechanism selected from the group consisting of: a buckle, a button, a chain, a clamp, a clasp, a clip, a hook, a hook-and-eye mechanism, a magnet, a pin, a plug, a snap, a strap, a string, a tie, a zipper, an adhesive, an elastic band, an electronic plug, insertion into a channel, insertion into a pocket, insertion into a pouch, and tape.

In an example, having modular neuromuscular activity (e.g. EMG) sensors which can be removably-attached to different locations on a band, strap, or sleeve can enable customizing sensor configuration to more accurately record the activity of different muscles at different times. In another example, one or more selected neuromuscular activity (e.g. EMG) sensors can be moved by one or more electromagnetic actuators to customize sensor configuration for a specific person or activity. In an example, the number, type, location, orientation, and/or configuration of EMG sensors on a band can be selected, configured, customized, and/or adjusted so as to best collect muscle activity data during a particular type of (athletic) activity.

In another example, a band, strap, or sleeve with neuromuscular activity (e.g. EMG) sensors can further comprise a plurality of electromagnetic actuators, wherein a selected subset of EMG sensors can be moved around (a portion of) the circumference of the band by activating a selected subset of actuators. In an example, a band, strap, or sleeve with neuromuscular activity (e.g. EMG) sensors can further comprise a track, channel, or ring around its inner perimeter, wherein one or more EMG sensors can be moved to different radial locations by sliding along the track, channel, or ring; wherein the track, channel, or ring spans the center of the width of the band.

In an example, a band, strap, or sleeve with neuromuscular activity (e.g. EMG) sensors can further comprise one or more circumferential tracks, channels, or rings around its inner perimeter, wherein one or more EMG sensors are automatically moved (e.g. slid) along the one or more tracks, channels, or rings by one or more electromagnetic actuators to compensate (e.g. correct) for erroneous data from one or more EMG sensors. In an example, a band, strap, or sleeve with neuromuscular activity (e.g. EMG) sensors can further comprise one or more circumferential tracks, channels, or rings around its inner perimeter, wherein one or more EMG sensors can be moved by sliding along the one or more tracks, channels, or rings.

In another example, a channel or track along which an EMG sensor slides can be parallel to a circumference of the band. In an example, a neuromuscular activity (e.g. EMG) sensor can be moved circumferentially around (a portion of) a person's wrist (and/or forearm) by moving (e.g. sliding) the sensor along a circumferential track or pathway. In an example, an EMG sensor can be moved around a portion of the inner perimeter of a band which is worn around a person's wrist and/or forearm. In an example, device can further comprise an electromagnetic actuator which automatically moves one or more neuromuscular activity (e.g. EMG) sensors around (a portion of) the circumference of a band.

In an example, the distances between sensors on a band can be selectively-adjusted by moving them along one or more channels, tracks, or rings. In one embodiment, the radial location of one or more EMG sensors on a person's wrist can be automatically adjusted by an electromagnetic actuator in response to lack of direct contact between the sensor and the person's wrist. In another example, the radial locations of EMG sensors on the circumference of a band can be selectively and independently adjusted by electromagnetic actuators on the band. In an example, a lateral channel or track on a band can span some (or all) of the width of a person's wrist (and/or forearm). In an example, a neuromuscular activity (e.g. EMG) sensor can slide along a lateral channel or track. In an example, device can further comprise an electromagnetic actuator which automatically moves one or more neuromuscular activity (e.g. EMG) sensors across a (portion of) the width of a band.

In another example, a band, strap, or sleeve with neuromuscular activity (e.g. EMG) sensors can comprise a first set of EMG sensors which are flat on the body-facing surface of the band and a second set of EMG sensors which protrude out from the body-facing surface of the band. In an example, a band, strap, or sleeve with neuromuscular activity (e.g. EMG) sensors can further comprise a plurality of electromagnetic actuators. wherein the actuators change the distance by which the EMG sensors protrude out from the body-facing surface of the band.

In an example, a selected subset of EMG sensors on a band can be automatically moved out from the band toward a person's wrist (and/or forearm) by an electromagnetic actuator (e.g. miniature electric motor). In an example, adjacent EMG sensors around the circumference of a band can be separated by different distances, wherein EMG sensors on a portion of the band which spans the ventral surface of a person's wrist are farther apart than EMG sensors on the rest of the band. In another example, EMG sensors on a band can protrude out from the body-facing surface of the band. In an example, the heights of EMG sensors on a band can be selectively and independently adjusted by electromagnetic actuators on the band.

In another example, the amount of pressure exerted by one or more EMG sensors on a person's wrist can be adjusted by an electromagnetic actuator. In an example, the amount of pressure exerted by one or more EMG sensors on a person's wrist can be automatically adjusted by an electromagnetic actuator in response to lack of electromagnetic communication between the sensor and the person's wrist. In an example, a selected set of one or more neuromuscular activity (e.g. EMG) sensors can be rotated. In one embodiment, an EMG sensor on a band can be automatically rotated by an electromagnetic actuator (e.g. miniature electric motor).

In an example, a neuromuscular activity (e.g. EMG) sensor on a band can be automatically moved by an electromagnetic actuator (e.g. miniature electric motor). In an example, an actuator on a band can be a brush-type DC motor. In an example, an actuator on a band can be an electromagnetic actuator. In another example, a selected set of one or more neuromuscular activity (e.g. EMG) sensors can be changed from a first configuration to a second configuration by activation of one or more hydraulic pistons or electromagnetic solenoids between the sensors and the band. In an example, the distance between a neuromuscular activity (e.g. EMG) sensor on a band and a person's wrist (and/or forearm) can be adjusted by activation of a piston or solenoid between the sensor and the band. In another example, the radial location of one or more EMG sensors on a person's wrist can be adjusted by an electromagnetic solenoid.

In an example, a band, strap, or sleeve can have two expandable sections can collectively span between 5% and 30% of the circumference of the person's wrist (and/or forearm). In another example, an EMG sensor can comprise a low-conductivity chamber and/or lumen which is filled with a high-conductivity fluid. In an example, the length (and fit) of a band or strap with neuromuscular activity (e.g. EMG) sensors can be adjusted via a plurality of expandable sections around the circumference of the band or strap. In an example, the pressure between a neuromuscular activity (e.g. EMG) sensor and a person's wrist (and/or forearm) can be adjusted by pumping a flowable substance into, or out of, an expandable chamber between the sensor and the band.

In one embodiment, a selected set of one or more neuromuscular activity (e.g. EMG) sensors can be changed from a first configuration to a second configuration by expansion (e.g. inflation) of one or more expandable chambers between the sensors and the band. In an example, the distance between a neuromuscular activity (e.g. EMG) sensor on a band and a person's wrist (and/or forearm) can be adjusted by inflation or deflation of an expandable chamber between the sensor and the band. In an example, the pressure between a neuromuscular activity (e.g. EMG) sensor and a person's wrist (and/or forearm) can be adjusted by inflation or deflation of an expandable chamber between the sensor and the band. In another example, the radial locations of EMG sensors on a band can be adjusted by one or more inflatable chambers between them and the main body of the band.

In an example, a band with neuromuscular activity (e.g. EMG) sensors can comprise two adjustable-length piezoelectric sections and a non-stretchable section, wherein the piezoelectric sections are closer to (e.g. adjacent to) a display screen (and/or watch housing) than the non-stretchable section. In another example, an actuator used in a band can be a piezoelectric actuator. In an example, the amount of pressure exerted by one or more EMG sensors on a person's wrist can be adjusted by a piezoelectric actuator. In another example, a band, strap, or sleeve with neuromuscular activity (e.g. EMG) sensors can further comprise one or more springs which exert compressive force on the circumference of the band. In an example, the extent to which one or more neuromuscular activity (e.g. EMG) sensors protrude out from a band can be selectively-adjusted by changing the tension of one or more springs.

In an example, a band or strap can have two expandable (e.g. telescoping) sections which connect the rest of the band to sides of a display screen (and/or watch housing). In an example, a band or strap with neuromuscular activity (e.g. EMG) sensors can have a plurality of electronics housings around at least half of its circumference, wherein these housings are interconnected by telescoping members. In one embodiment, a section of a band with an adjustable length can comprise telescoping components. In an example, the length of a band around a person's wrist (and/or forearm) can be adjusted by moving telescoping components on the band relative to each other. In another example, a band, strap, or sleeve with neuromuscular activity (e.g. EMG) sensors can further comprise a data transmitter. In an example, a band, strap, or sleeve with neuromuscular activity (e.g. EMG) sensors can include one or more electronic circuits. In another example, device can further comprise a battery, an amplifier, a data processor, and/or a data transceiver.

In an example, a band with EMG sensor can further an integrated display screen. In another example, a band, strap, or sleeve with neuromuscular activity (e.g. EMG) sensors can further comprise a touchscreen. In an example, a display screen can be integrated into a wearable device (e.g. band, watch, or watch band) with neuromuscular activity (e.g. EMG) sensors. In an example, a wearable computing device for the wrist and/or arm can comprise a hexagonal display screen.

In an example, an EMG sensor can be made by melting or adhering elastomeric conductive material onto a low-conductivity textile or fabric. In an example, an EMG sensor can be made from polydimethylsiloxane (PDMS) which has been doped, impregnated, and/or coated with carbon nanotubes. In one embodiment, an EMG sensor can be made with a high-conductivity material selected from the group consisting of: aluminum or aluminum alloy; carbon nanotubes, graphene, or other carbon-based material; copper or copper alloy; gold; nickel; silver; and steel. In another example, an EMG sensor can be made with polydimethylsiloxane (PDMS) which has been doped or impregnated with aluminum, carbon (in one or more various configurations and formulations), copper, gold, nickel, silver, and/or steel. In an example, an EMG sensor can have a low-conductivity (or non-conductive) layer made from acetate, acrylic, cotton, denim, elastane, latex, linen, Lycra, neoprene, nylon, nylon, polyester, wool, silicone, polydimethylsiloxane (PDMS), silk, spandex, or rayon. In another example, an EMG sensor can include a layer made from thermoplastic polymer. In an example, an EMG sensor can comprise a portion of fabric (e.g. a textile) in which electroconductive yarns, threads, or filaments have been woven.

In another example, a band, strap, or sleeve with neuromuscular activity (e.g. EMG) sensors can further comprise one or more accelerometers. In an example, a band, strap, or sleeve with neuromuscular activity (e.g. EMG) sensors can further comprise one or more motion sensors. In an example, data from both EMG sensors and inertial motion sensors can be jointly analyzed in order to measure and/or model body motion and/or muscle activity. In an example, a band, strap, or sleeve with neuromuscular activity (e.g. EMG) sensors can further comprise a camera on the portion of the band which spans the ventral surface of a person's wrist. In an example, a band, strap, or sleeve with neuromuscular activity (e.g. EMG) sensors can further comprise a heart rate sensor. In an example, a band, strap, or sleeve with neuromuscular activity (e.g. EMG) sensors can further comprise one or more haptic and/or tactile actuators.

In another example, a band, strap, or sleeve with neuromuscular activity (e.g. EMG) sensors and an integrated display screen can function as a human-to-computer interface. In an example, a band, strap, or sleeve with neuromuscular activity (e.g. EMG) sensors can function as a Human-to-Computer control interface. In another example, data from EMG sensors on a band can be analyzed to recognize selected hand gestures, wherein these hand gestures control the operation of a separate device. In one embodiment, data from EMG sensors on a band can be analyzed to recognize selected hand gestures.

In another example, a band or sleeve which functions as a watch strap can span between 50% and 90% of the circumference of a person's wrist (and/or forearm). In an example, a band, strap, or sleeve with neuromuscular activity (e.g. EMG) sensors can be a watch band. In an example, a band, strap, or sleeve with neuromuscular activity (e.g. EMG) sensors can comprise a wrist band. In an example, a band, strap, or sleeve with neuromuscular activity (e.g. EMG) sensors which functions as a watch strap which can hold different types and/or brands of watch housings on a person's wrist. In an example, a watch housing can be removably-attached to a band, strap, or sleeve with neuromuscular activity (e.g. EMG) sensors.

In another example, a band, strap, or sleeve with neuromuscular activity (e.g. EMG) sensors and an integrated display screen can function generally as a wearable computing devices with sensors and a screen. In an example, a band, strap, or sleeve with neuromuscular activity (e.g. EMG) sensors can comprise a sleeve or cuff. In another example, neuromuscular activity (e.g. EMG) sensors can be distributed around the inner circumference of a shirt sleeve (or cuff). In an example, ends of a band can be connected by Velcro. In an example, the ends of a band, strap, or sleeve with neuromuscular activity (e.g. EMG) sensors can be attached to each other by magnetism.

In one embodiment, a watch band with neuromuscular activity (e.g. EMG) sensors can have an adjustable length (e.g. partial circumference); wherein the watch band is connected to the watch housing by two components (one on each side of the watch housing); wherein the two ends of the watch band are inserted and reversed in orientation through these two components, respectively, to overlap the rest of the watch band; and wherein the length of the watch band is adjusted by changing the amount by which the two ends of the watchband inserted through the two components overlap the rest of the watch band.

In an example, a band or strap with neuromuscular activity (e.g. EMG) sensors can have one or more housings in which one or more ends of the band are coiled, wherein the length of the band around a person's wrist (and/or forearm) can be adjusted by changing the amounts of the one or more ends which are coiled within the housing. In another example, an end of a band can curve (e.g. loop) around a roller member (e.g. axle) in a coil housing to double back on (and overlap a portion of) the rest of the band. In one embodiment, the ends of a band (or strap or sleeve) with neuromuscular activity (e.g. EMG) sensors can be connected to a watch housing, wherein the ends of the band roll around cylindrical components where they connect to the watch housing so as to overlap portions of the rest of the band.

In another example, the ends of a band (or strap or sleeve) with neuromuscular activity (e.g. EMG) sensors can be connected to a watch housing, wherein the ends of the band roll around cylindrical components where they connect to the watch housing so as to overlap portions of the rest of the band, wherein the fit (e.g. tightness or looseness) of the band around the person's wrist can be adjusted by adjusting (e.g. changing) the amount by which the ends overlap the rest of the band, wherein the band can be made tighter by increasing the amount of overlap between the ends of the band and the rest of the band, and wherein the band can be made looser by decreasing the amount of overlap between the ends of the band and the rest of the band.

In an example, a band or strap with neuromuscular activity (e.g. EMG) sensors can comprise a plurality of stretchable and/or elastic sections. In an example, a band, strap, or sleeve can have two elastic and/or stretchable sections which connect to a display screen (and/or watch housing). In an example, a band, strap, or sleeve with neuromuscular activity (e.g. EMG) sensors can have a variable circumference, wherein there are two elastic and/or stretchable sections on the circumference of the band. In an example, a watch band with neuromuscular activity (e.g. EMG) sensors can have a variable length (e.g. partial circumference), wherein there are two elastic and/or stretchable sections on the watch band, and where these two section are on either side of a watch housing. In an example, the elasticity level of a band can be adjusted.

In an example, a band, strap, or sleeve with neuromuscular activity (e.g. EMG) sensors can have an adjustable circumference. In another example, the circumference of a band can be adjusted. In an example, the fit (e.g. tightness or looseness) of a band, strap, or sleeve with neuromuscular activity (e.g. EMG) sensors can be adjusted by changing the amount of overlap between one or both end portions of the band and the rest of the band.

In another example, a band, strap, or sleeve with neuromuscular activity (e.g. EMG) sensors can be a watch strap which is wider than the watch housing. In one embodiment, a maximum (proximal-to-distal) width of a band, strap, or sleeve can be at least 20% greater than a maximum (proximal-to-distal) width of a display screen (and/or watch housing) on the band, strap, or sleeve. In an example, a portion of a band, strap, or sleeve which spans the ventral surface of a person's wrist (and/or forearm) can be wider than the width of a watch housing. In one embodiment, the average (proximal-to-distal) width of a band, strap, or sleeve can be at least 20% greater than the average (proximal-to-distal) width of a display screen (and/or watch housing). In an example, the maximum width of a first portion of a band which spans the ventral surface of a person's wrist or forearm and be at least 25% wider than the maximum width of a second portion of the band which spans the dorsal surface of the person's wrist or forearm.

In another example, a band, strap, or sleeve can have a (sinusoidal) undulations as it curves around (a portion of) the circumference of a person's wrist (and/or forearm). In an example, a band, strap, or sleeve with neuromuscular activity (e.g. EMG) sensors can comprise a series of convex and concave segments. In another example, there can be a (partial) circumferential array (e.g. a ring) of holes around the (partial) circumference of a band or strap.

In an example, a bifurcating band can bifurcate into two bands on the dorsal surface of a person's wrist (and/or forearm), forming two (parallel) branches which span the lateral and ventral surfaces of the wrist (and/or forearm). In another example, a dual band with neuromuscular activity (e.g. EMG) sensors can comprise a proximal band and a distal band, wherein both bands are around at least 50% of a circumference of a person's wrist (and/or forearm), and wherein the proximal band is closer to the person's wrist (and/or forearm) than the distal band. In an example, a proximal band can be inserted into, under, or over a distal band to a first extent in the first configuration and the proximal band can be inserted into, under, or over the distal band to a second extent in the second configuration, wherein the second extent is less than the first extent. In an example, a proximal band can is closer to the person's shoulder and a distal band can be farther from the person's shoulder.

In an example, a band or strap with neuromuscular activity (e.g. EMG) sensors can comprise an alternating series of rigid components and flexible (e.g. elastic or stretchable) components, wherein the series alternates between rigid and flexible components around (at least a portion of) the circumference of a person's wrist (and/or forearm). In an example, a band with neuromuscular activity (e.g. EMG) sensors can comprise an alternating sequence of rigid (e.g. polymer or metal) housings and stretchable (e.g. fabric) sections around a person's wrist (and/or forearm), wherein the rigid sections collectively span at least 20% more of the circumference of the wrist (and/or forearm) than the stretchable sections.

In one embodiment, a band with neuromuscular activity (e.g. EMG) sensors can comprise an alternating sequence of rigid (e.g. polymer or metal) housings and stretchable (e.g. fabric) sections around a person's wrist (and/or forearm), wherein the stretchable sections collectively span at least 20% more of the circumference of the wrist (and/or forearm) than the rigid sections. In an example, a band, strap, or sleeve with neuromuscular activity (e.g. EMG) sensors can comprise a single piece of flexible material. In another example, a band, strap, or sleeve with neuromuscular activity (e.g. EMG) sensors can have a plurality of electronics housings around at least half of its circumference, wherein EMG sensors are housed in these housings. In one embodiment, neuromuscular activity (e.g. EMG) sensors can be located on rigid segments of a band. In another example, a band, strap, or sleeve with neuromuscular activity (e.g. EMG) sensors can comprise a ring of fabric between two polymer bands around a person's wrist and/or forearm. In an example, circumferential sections of band, strap, or sleeve which is worn on a person's wrist (and/or forearm) can be made from a breathable fabric and/or textile.

In another example, a band, strap, or sleeve with neuromuscular activity (e.g. EMG) sensors which serves as a watch strap can be in direct (e.g. plug in) electromagnetic communication with electronic components in a watch housing. In an example, a battery, and/or data processing components in a watch housing can be in electromagnetic communication and/or power transmission connection with neuromuscular activity (e.g. EMG) sensors on a band. In an example, stretchable sections of a band can have flexible electroconductive pathways (e.g. sinusoidal wires or electroconductive threads) which transmit power to rigid housings and/or transmit data from the sensors.

In an example, an EMG sensor can be a capacitive electromyography sensor In an example, an EMG sensor can be a surface EMG sensor. In an example, neuromuscular activity sensors can be electromyography (e.g. EMG) sensors. In another example, a band, strap, or sleeve with neuromuscular activity (e.g. EMG) sensors can comprise a plurality of paired electrodes on the inner circumference of the band. In an example, an EMG sensor can comprise a pair of electrodes.

In another example, an EMG sensor can be a capacitive sensor with a first layer comprising less electroconductive (and/or non-conductive) material and a second layer of more electroconductive material, wherein the first layer is closer to the surface of a person's body than the second layer. In an example, an EMG sensor can comprise a first layer of material with a first level of permittivity and a second layer of material with a second level of permittivity, wherein the first layer is closer to the surface of the person's body than the second layer. In another example, an EMG sensor can comprise an electroconductive layer which is made from a silicon-based polymer which has been doped, impregnated, and/or coated with conductive material. In an example, an EMG sensor can comprise an electroconductive layer which is made from an elastomeric polymer which has been doped, impregnated, and/or coated with carbon nanotubes. In an example, an EMG sensor can comprise an electroconductive layer which is made from PDMS which has been doped, impregnated, and/or coated with conductive material. In one embodiment, an EMG sensor can include an electroconductive substrate.

In an example, an EMG sensor can comprise a dielectric layer of low-conductivity material between two layers of high-conductivity material. In an example, an EMG sensor can comprise a first layer of dielectric material which is closer to the person's body and a second layer of electroconductive material which is farther from the person's body. In an example, an EMG sensor can comprise an electroconductive layer (e.g. plate) which is coated with non-electroconductive material. In another example, the fabric (e.g. textile) of a shirt can function as a dielectric layer for capacitive EMG sensors in the shirt. In an example, EMG sensors on a band can have one or more Shore OO values between 10 and 30.

In another example, a band, strap, or sleeve with neuromuscular activity (e.g. EMG) sensors can have EMG sensors with frustal shapes which protrude out from the inner circumference of the band toward the surface of a person's wrist and/or forearm. In an example, an EMG sensor can have a shape selected from the group consisting of: hemispherical, rectangular, rounded-rectangular, circular, trapezoidal, and elliptical. In another example, an EMG sensor on a band can have a hemispherical shape. In an example, an EMG sensor on a band can have a rounded-square shape. In an example, EMG sensors on a band can be flat on the body-facing surface of the band. In an example, neuromuscular activity sensors can have hemispherical or half-cylindrical shapes.

In an example, the body-facing surfaces of EMG sensors in a band can be arcuate. In an example, EMG sensors around the circumference of a band can have different sizes, wherein EMG sensors on a portion of the band which spans the ventral surface of a person's wrist are smaller than EMG sensors on the rest of the band. In another example, an EMG sensor can be made by printing electroconductive ink on fabric. In one embodiment, neuromuscular activity (e.g. EMG) sensors can be formed by printing electroconductive ink onto a sleeve (or cuff). In another example, neuromuscular activity (e.g. EMG) sensors can be attached to a sleeve by sewing or embroidering. In an example, neuromuscular activity (e.g. EMG) sensors can be formed by weaving, sewing, braiding, or embroidering electroconductive yarns, threads, or fibers into the fabric used to create a sleeve (or cuff).

In an example, an array of EMG sensors on a band can comprise two circumferential rings. In an example, EMG sensors on a band can be configured in nested (e.g. concentric) rings. In an example, neuromuscular activity (e.g. EMG) sensors on a band can be aligned along a single (e.g. proximally-to-distal central) circumferential ring around the person's wrist (and/or forearm). In an example, pairs of EMG sensors around the circumference of a band can be pair-wise colinear. In an example, EMG sensors can be on the inner circumference of a band and collectively span between 40% and 70% of the circumference of a wrist or forearm to which the band is attached. In another example, EMG sensors on a band can collectively span between 75% and 90% of the circumference of a wrist or forearm.

In an example, between 40% and 60% of EMG sensors on band can be configured to contact the ventral side of a wrist or forearm. In another example, EMG sensors on a portion of a band which spans the ventral surface of a person's wrist can be less densely-distributed than EMG sensors on the rest of the band. In an example, EMG sensors on a portion of a band which spans the ventral surface of a person's wrist can protrude out less from the band than EMG sensors on the rest of the band. In another example, neuromuscular activity (e.g. EMG) sensors can be on ventral and lateral portions of the perimeter of a band, wherein a ventral portion is the portion which spans the ventral surface of the person's wrist (and/or forearm) and the lateral portions span the sides of the person's wrist (and/or forearm) between ventral and dorsal surfaces of the person's wrist (and/or forearm). In an example, EMG sensors can be evenly spaced around the entire circumference of a person's wrist or forearm. In an example, a longitudinal axis of a longitudinal neuromuscular activity (e.g. EMG) sensor can be orthogonal to a circumference of a band.

In one embodiment, a band, strap, or sleeve with modular neuromuscular activity (e.g. EMG) sensors can be created by attaching, clipping, connecting, plugging, inserting, and/or snapping modular sensors onto the band, strap, or sleeve at different locations at different times. In an example, a band, strap, or sleeve with neuromuscular activity (e.g. EMG) sensors can have a ring-and-row array of attachment locations (e.g. ports) to which one or more modular EMG sensors can be removably attached. In an example, modular neuromuscular activity (e.g. EMG) sensors can be attached to a band, strap, or sleeve by pins or snaps.

In an example, a customized article of clothing with modular neuromuscular activity (e.g. EMG) sensors can be created by attaching, clipping, connecting, plugging, inserting, and/or snapping modular sensors onto the article of clothing at selected locations and/or different locations at different times. In another example, modular neuromuscular activity (e.g. EMG) sensors which can be removably-attached to different locations on a band, strap, or sleeve can enable customizing the configuration of sensors to monitor a selected set of muscles used in a selected activity. In an example, selected sensors on a band can be moved laterally and/or radially in order to customize sensor configuration for a specific sport of activity. In another example, the number, type, location, orientation, and/or configuration of EMG sensors on a band can be selected, configured, customized, and/or adjusted so as to best collect muscle activity data for a specific person.

In an example, a band, strap, or sleeve with neuromuscular activity (e.g. EMG) sensors can further comprise a plurality of electromagnetic actuators, wherein the actuators change the radial locations of the EMG sensors on the circumference of the band, and wherein there is a one-to-one correspondence between EMG sensors and actuators. In another example, a band, strap, or sleeve with neuromuscular activity (e.g. EMG) sensors can further comprise a track, channel, or ring around its inner perimeter, wherein one or more EMG sensors can be moved to different radial locations by sliding along the track, channel, or ring.

In an example, a band, strap, or sleeve with neuromuscular activity (e.g. EMG) sensors can further comprise one or more circumferential tracks, channels, or rings around its inner perimeter, wherein one or more EMG sensors are automatically moved (e.g. slid) along the one or more tracks, channels, or rings by one or more electromagnetic actuators to compensate (e.g. correct) for rotation of the band around a person's wrist and/or forearm. In an example, a band, strap, or sleeve with neuromuscular activity (e.g. EMG) sensors can further comprise two parallel tracks, channels, or rings around its inner perimeter, wherein one or more EMG sensors can be moved by one or more electromagnetic actuators to different radial locations by sliding along the tracks, channels, or rings.

In an example, a circumferential channel or track on a band can span some (or all) of the circumference of a person's wrist (and/or forearm). In an example, a selected subset of EMG sensors on a band can be automatically moved around a portion of the inner circumference of the band by an electromagnetic actuator (e.g. miniature electric motor). In an example, an EMG sensor on a band can be automatically moved around a portion of the inner circumference of the band by an electromagnetic actuator (e.g. miniature electric motor).

In another example, device can further comprise an electromagnetic actuator which automatically moves one or more neuromuscular activity (e.g. EMG) sensors around (a portion of) the circumference of a person's wrist (and/or forearm). In one embodiment, the radial location of one or more EMG sensors on a person's wrist can be adjusted by an electromagnetic actuator. In another example, the radial location of one or more EMG sensors on a person's wrist can be automatically adjusted by an electromagnetic actuator in response to lack of electromagnetic communication between the sensor and the person's wrist. In an example, the radial locations of one or more EMG sensors on the circumference of a band can be adjusted.

In an example, a lateral channel or track on a band can span some (or all) of the width of the band. In an example, a selected subset of EMG sensors on a band can be automatically moved across a portion of the width of the band by an electromagnetic actuator (e.g. miniature electric motor). In an example, device can further comprise an electromagnetic actuator which automatically moves one or more neuromuscular activity (e.g. EMG) sensors across a (portion of) the width of a person's wrist (and/or forearm)

In an example, a band, strap, or sleeve with neuromuscular activity (e.g. EMG) sensors can further comprise a plurality of (electromagnetic) actuators, wherein the actuators change the distance by which the EMG sensors protrude out from the body-facing surface of the band, and wherein there is a one-to-one correspondence between EMG sensors and actuators. In another example, a band, strap, or sleeve with neuromuscular activity (e.g. EMG) sensors can include one or more electromagnetic actuators which change the distances by which one or more sensors protrude out from the band toward the wrist.

In an example, a wearable device (e.g. a band, smart watch, or watch band) with neuromuscular activity (e.g. EMG) sensors can comprise: a band, strap, or sleeve which is configured to be worn around at least 50% of a circumference of a person's wrist (and/or forearm); and a plurality of helically-threaded neuromuscular activity (e.g. EMG) sensors on the band, wherein rotation of a helically-threaded neuromuscular activity (e.g. EMG) sensor changes the distance by which the sensor protrudes out from the band toward the person's wrist (and/or forearm).

In another example, an EMG sensor on a band can be automatically moved out from the band toward a person's wrist (and/or forearm) by an electromagnetic actuator (e.g. miniature electric motor). In an example, the distances between EMG sensors and the main body of a band can be adjusted by changing the tensions of springs between them and the main body of the band. In another example, when signals from one or more EMG sensors on a band are weak or inaccurate, then distances between those EMG sensors and the main body of the band can be automatically adjusted by one or more electromagnetic actuators.

In an example, the amount of pressure exerted by one or more EMG sensors on a person's wrist can be adjusted. In an example, the proximity and/or force of one or more EMG sensors relative to a wrist or forearm can be changed by one or more hydraulic mechanisms. In one embodiment, a selected subset of EMG sensors on a band can be automatically rotated by an electromagnetic actuator (e.g. miniature electric motor). In an example, the amount by which an EMG sensor with helical threads which protrudes out from a band toward a person's wrist and/or forearm can be adjusted by rotating the EMG sensor through a threaded opening in the band. In another example, a selected subset of neuromuscular activity (e.g. EMG) sensors on a band can be automatically moved by an electromagnetic actuator (e.g. miniature electric motor). In an example, an actuator on a band can be a hydraulic actuator. In another example, one or more actuators on a band (or strap) can contract or expand when activated.

In an example, a wearable device (e.g. a band, smart watch, or watch band) with neuromuscular activity (e.g. EMG) sensors can comprise: a band, strap, or sleeve which is configured to be worn around at least 50% of a circumference of a person's wrist (and/or forearm); a plurality of neuromuscular activity (e.g. EMG) sensors on the band; a plurality of hydraulic pistons or electromagnetic solenoids between the sensors and the band, wherein selective activation of one or more of the pistons or solenoids pushes one or more of the sensors away from the band toward the person's wrist (and/or forearm). In an example, the extent to which one or more neuromuscular activity (e.g. EMG) sensors protrude out from a band can be selectively-adjusted by activation of hydraulic pistons or electromagnetic solenoids.

In an example, a band or strap with neuromuscular activity (e.g. EMG) sensors can comprise a plurality of expandable sections. In an example, a band, strap, or sleeve with neuromuscular activity (e.g. EMG) sensors can include one or more expandable (e.g. fluid-filled) chambers which change the distances by which one or more sensors protrude out from the band toward the wrist. In an example, expandable section of a band or strap can be made with elastic and/or stretchable material. In an example, the length of an expandable section of a band can be adjusted by an electromagnetic actuator. In another example, the proximity and/or force of one or more EMG sensors relative to a wrist or forearm can be changed by filling one or more expandable chambers with a flowable substance (e.g. fluid or gas).

In an example, a wearable device (e.g. a band, smart watch, or watch band) with neuromuscular activity (e.g. EMG) sensors can comprise: a band, strap, or sleeve which is configured to be worn around at least 50% of a circumference of a person's wrist (and/or forearm); a plurality of neuromuscular activity (e.g. EMG) sensors on the band; and a plurality of expandable (e.g. inflatable) chambers between the sensors and the band, wherein selective expansion (e.g. inflation) of one or more of the expandable chambers pushes one or more of the sensors away from the band toward the person's wrist (and/or forearm). In another example, the extent to which one or more neuromuscular activity (e.g. EMG) sensors protrude out from a band can be selectively-adjusted by expansion of one or more expandable (e.g. inflatable) chambers. In an example, the proximity and/or force of one or more EMG sensors relative to a wrist or forearm can be changed by inflating or deflating one or more inflatable chambers.

In one embodiment, a band or strap with neuromuscular activity (e.g. EMG) sensors can comprise a plurality of piezoelectric sections. In an example, a band, strap, or sleeve with neuromuscular activity (e.g. EMG) sensors can further comprise a plurality of piezoelectric sensors. In an example, an expandable section of a band can be piezoelectric, wherein the section is expanded or contracted by the transmission of electrical energy through the section. In an example, the length (and fit) of a band or strap with neuromuscular activity (e.g. EMG) sensors can be adjusted via one or more piezoelectric sections around the circumference of the band or strap. In another example, a band, strap, or sleeve with neuromuscular activity (e.g. EMG) sensors can have a plurality of electronics housings around at least half of its circumference, wherein these housings are interconnected by springs. In an example, the proximity and/or force of one or more EMG sensors relative to a wrist or forearm can be changed by adjusting one or more spring mechanisms.

In another example, a band or strap with neuromuscular activity (e.g. EMG) sensors can comprise a plurality of telescoping sections. In an example, a band, strap, or sleeve with neuromuscular activity (e.g. EMG) sensors can comprise an alternating circumferential (or partially-circumferential) series of electronic housings (wherein each housing contains one or more EMG sensors) and telescoping components (which are compelled apart by tension from internal springs), wherein the series alternates (back and forth) between electronic housings and telescoping components around (a portion of) the circumference of the band. In another example, telescoping members can be held in tension by springs. In an example, the size and/or tension of the circumference of a band can be changed by adjusting a telescoping section on this circumference.

In an example, a band, strap, or sleeve with neuromuscular activity (e.g. EMG) sensors can include a multiplexer. In an example, a wearable computing device for the wrist and/or arm can comprise: a band, strap, or sleeve; a display screen; a plurality of neuromuscular activity (e.g. EMG) sensors; a battery; an amplifier; a data processor; and a data transmitter. In an example, a band, strap, or sleeve with neuromuscular activity (e.g. EMG) sensors can further comprise a battery.

In one embodiment, a band, strap, or sleeve with neuromuscular activity (e.g. EMG) sensors can be a separate device from a display screen, to which different types and brands of display screens can be removably-attached. In another example, a band, strap, or sleeve with neuromuscular activity (e.g. EMG) sensors can hold a circular display screen and/or watch housing on a person's wrist. In an example, a wearable computing device for the wrist and/or arm can comprise a flexible display screen. In another example, display screen (and/or watch housing) can be a separate device which can be removably-attached to the band.

In an example, an EMG sensor can be made from conductive core yarn, copper thread coated with polyester, polyester yarn coated with metal, steel fiber yarn, synthetic filament fiber yarn, yarn coated with carbon, yarn coated with copper, or yarn coated with silver. In an example, an EMG sensor can be made from polydimethylsiloxane (PDMS) which has been doped, impregnated, and/or coated with silver. In an example, an EMG sensor can be made with a low-conductivity material which has been doped, impregnated, or coated with high-conductivity material. In an example, an EMG sensor can comprise two electrodes which are made from gold, silver, copper, steel, titanium, electrically-conductive rubber, or electrically-conductive silicone.

In an example, an EMG sensor can include a layer made from a thermoplastic polymer which has been doped, impregnated, and/or coated with electroconductive material (e.g. carbon black, silver particles, or aluminum particles). In another example, electroconductive threads, fibers, yarns, strands, filaments, traces, layers, inks, and/or resins in a band can be made from one or more materials selected from the group consisting of: aluminum (Al), aluminum alloy, brass (Ms), carbon nanotubes, carbon-based material, ceramic particles, copper (Cu), copper alloy, copper-clad aluminum, fluorine, gold (Au), graphene, magnesium, nickel, niobium (Nb), organic solvent, polyaniline, polymer, rubber, silicone, silver (Ag), silver chloride (AgCI), silver-plated brass (Ms/Ag), silver-plated copper (Cu/Ag), and steel. In an example, an EMG sensor can comprise a portion of fabric (e.g. a textile) with an array of electroconductive yarns, threads, or filaments.

In another example, a band, strap, or sleeve with neuromuscular activity (e.g. EMG) sensors can further comprise one or more gyroscopes. In another example, a band, strap, or sleeve with neuromuscular activity (e.g. EMG) sensors can further comprise one or more strain, bend, and/or stretch sensors. In an example, data from both EMG sensors and strain-based motion sensors can be jointly analyzed in order to measure and/or model body motion and/or muscle activity. In an example, a band, strap, or sleeve with neuromuscular activity (e.g. EMG) sensors can further comprise a camera which is triggered (e.g. activated) by a selected hand gesture detected based on data from the EMG sensors. In an example, a band, strap, or sleeve with neuromuscular activity (e.g. EMG) sensors can further comprise a temperature sensor.

In one embodiment, a band, strap, or sleeve with neuromuscular activity (e.g. EMG) sensors can be used for gesture recognition. In an example, a band, strap, or sleeve with neuromuscular activity (e.g. EMG) sensors can record data which is used to measure, monitor, record, and/or recognize wrist, hand, and/or finger gestures. In an example, data from EMG sensors on a band can be analyzed to recognize selected hand gestures, wherein these hand gestures control the operation of augmented reality eyewear. In another example, data from EMG sensors on a wrist-worn band can be analyzed to monitor, estimate, model, and/or recognize finger and hand orientations and configurations.

In an example, a band or strap with neuromuscular activity (e.g. EMG) sensors can function as a watch strap. In another example, a band, strap, or sleeve with neuromuscular activity (e.g. EMG) sensors can be a watch strap which spans between 50% and 75% of the circumference of a person's wrist. In another example, a band, strap, or sleeve with neuromuscular activity (e.g. EMG) sensors can function as a watch band. In an example, a band, strap, or sleeve with neuromuscular activity (e.g. EMG) sensors which serves as a watch strap can be a continuous band, wherein the two ends of the band are connected to (the two opposite sides of) a watch housing.

In an example, a band, strap, or sleeve with neuromuscular activity (e.g. EMG) sensors and an integrated display screen can function as a smart watch. In an example, a forearm-worn computing device and/or motion recognition device with a display screen can be more generic than that which is currently categorized as a smart watch. In an example, neuromuscular activity (e.g. EMG) sensors can be formed by adhering electroconductive strips or patches onto a sleeve (or cuff). In one embodiment, a band, strap, or sleeve with neuromuscular activity (e.g. EMG) sensors can comprise a bracelet. In another example, the ends of a band, strap, or sleeve with neuromuscular activity (e.g. EMG) sensors can be attached to each other by a buckle or snap. In an example, the ends of a band, strap, or sleeve with neuromuscular activity (e.g. EMG) sensors can be connected to each other on the ventral surface of a person's wrist and/or forearm. In another example, the length of the end of a band which doubles back and overlaps the rest of the band can be adjusted.

In another example, a band or strap with neuromuscular activity (e.g. EMG) sensors can have two housings attached to two sides, respectively, of a display screen (and/or watch housing), wherein the two ends of a band are inserted and coiled within the housings, and wherein the length of the band around a person's wrist (and/or forearm) can be adjusted by changing the amount of the ends which are coiled within the housings. In an example, an end of band can be adjustably-coiled within a housing by an actuator in response to data from sensors on the band.

In an example, the ends of a band (or strap or sleeve) with neuromuscular activity (e.g. EMG) sensors can be connected to a watch housing, wherein the ends of the band roll around cylindrical components where they connect to the watch housing so as to overlap portions of the rest of the band, and wherein the length of the band around the person's wrist can be adjusted by adjusting (e.g. changing) the amount by which the ends overlap the rest of the band. In an example, the fit (e.g. tightness or looseness) of a band (or strap or sleeve) with neuromuscular activity (e.g. EMG) sensors can be adjusted by changing the amount of overlap between one or both end portions of the band and the rest of the band, wherein end portions of the band connect to a watch housing on the dorsal surface of a person's wrist (or forearm), wherein at least one end portion of the band curves (e.g. wraps or rolls, I love wrap and roll!) around a cylindrical component where it attached to the watch face to overlap a portion of the rest of the band, and wherein the fit of the band is adjusted by changing the amount (e.g. length) by which the at least one end portion overlaps the rest of the band.

In another example, a band with neuromuscular activity (e.g. EMG) sensors can comprise three stretchable (e.g. elastic) sections and two non-stretchable sections around the circumference of a person's wrist (and/or forearm). In an example, a band, strap, or sleeve with neuromuscular activity (e.g. EMG) sensors can further comprise one or more elastic sections which exert compressive force on the circumference of the band. In an example, a band, strap, or sleeve with neuromuscular activity (e.g. EMG) sensors can have non-uniform elasticity levels around its circumference. In an example, the length (and fit) of a band or strap with neuromuscular activity (e.g. EMG) sensors can be adjusted via a plurality of stretchable and/or elastic sections around the circumference of the band or strap.

In an example, the elasticity of a band can be automatically adjusted based on analysis of data from EMG sensors. In an example, one or more actuators can automatically adjust the fit of a band, strap, or sleeve with neuromuscular activity (e.g. EMG) sensors on a person's wrist and/or forearm. In one embodiment, the diameter of a band can be adjusted. In another example, the size and/or tension of the circumference of a band can be adjusted by an electromagnetic actuator.

In an example, a band, strap, or sleeve with neuromuscular activity (e.g. EMG) sensors can function as a watch strap, wherein the portion of the band, strap, or sleeve which spans the ventral surface of a person's wrist has a first average width, wherein the portions of the band, strap, or sleeve which span the lateral surfaces of the person's wrist have a second average width, and wherein the first average width is greater than the second average width. In another example, a maximum (proximal-to-distal) width of a band, strap, or sleeve can be at least 50% greater than a maximum (proximal-to-distal) width of a display screen (and/or watch housing) on the band, strap, or sleeve. In an example, EMG sensors can be located on wide sections of a band. In an example, the average width of a ventral portion of a band can be 30% to 80% greater than the average width of a dorsal portion of the band. In an example, the widths of the ends of a band or strap can taper toward where they connect to a display screen (and/or watch housing). In an example, an array of EMG sensors on a band can have (e.g. sinusoidal) undulations. In another example, EMG sensors can be located on concave segments of a band.

In an example, a band with neuromuscular activity (e.g. EMG) sensors can bifurcate into two branches on a dorsal surface of a person's wrist (and/or forearm). In another example, a dual band or strap with neuromuscular activity (e.g. EMG) sensors can comprise proximal and distal undulating (e.g. sinusoidal) bands with different phases and/or amplitudes. In an example, a dual band with neuromuscular activity (e.g. EMG) sensors can comprise a proximal band and a distal, wherein the proximal band is closer to the person's wrist (and/or forearm) than the distal band, and wherein a display screen (and/or watch housing) is attached to the distal band. In an example, a proximal band can be reversibly slid under (or out from under) a distal band. In one embodiment, a proximal band can overlap a distal band to a first extent in the first configuration and the proximal band can overlap the distal band to a second extent in the second configuration, wherein the second extent is less than the first extent.

In an example, a band or strap with neuromuscular activity (e.g. EMG) sensors can function as a watch strap with an alternating series of electronic housings (wherein each housing contains one or more EMG sensors) and elastic (e.g. stretchable fabric) sections wherein the series alternates (back and forth) between electronic housings and elastic sections along the strap. In an example, a band with neuromuscular activity (e.g. EMG) sensors can comprise an alternating sequence of rigid (e.g. polymer or metal) housings and stretchable (e.g. fabric) sections around a person's wrist (and/or forearm), wherein the rigid sections collectively span at least 50% more of the circumference of the wrist (and/or forearm) than the stretchable sections.

In an example, a band, strap, or sleeve can comprise a series of flexibly-connected rigid components (e.g. links or housings). In an example, a band, strap, or sleeve with neuromuscular activity (e.g. EMG) sensors can comprise an alternating circumferential (or partially-circumferential) series of electronic housings (wherein each housing contains one or more EMG sensors) and elastic (e.g. stretchable fabric) sections wherein the series alternates (back and forth) between electronic housings and elastic sections around (a portion of) the circumference of the band. In an example, a plurality of electronics housings around the perimeter of a band, strap, or sleeve with neuromuscular activity (e.g. EMG) sensors can be connected by a single elastic strip and/or strap. In an example, a band or sleeve, strap, or sleeve which is worn on a person's wrist (and/or forearm) can be made from a breathable fabric and/or textile. In another example, a band, strap, or sleeve with neuromuscular activity (e.g. EMG) sensors can comprise low-conductivity fibers and highly-conductive fibers which are braided or woven together.

In an example, a band, strap, or sleeve with neuromuscular activity (e.g. EMG) sensors can comprise a circumferential array of rigid components which are flexibly-connected by stretchable, elastic, and/or bendable sections; wherein the stretchable, elastic, and/or bendable sections further comprise flexible (e.g. undulating) electroconductive pathways which transmit power and/or electronic data between the rigid components. In another example, a band, strap, or sleeve with neuromuscular activity (e.g. EMG) sensors which serves as a watch strap can be in electromagnetic communication with electronics in a watch housing. In an example, a battery, and/or data processing components in a watch housing can be in electromagnetic communication and/or power transmission connection with neuromuscular activity (e.g. EMG) sensors via flexible electroconductive pathways. In an example, there can be a direct electromagnetic connection (e.g. plug, port, and/or outlet) between a band, strap, or sleeve with neuromuscular activity (e.g. EMG) sensors which serves as a watch strap and a watch housing which is held on a wrist by the band.

In one embodiment, an EMG sensor can be a capacitive sensor. In an example, an EMG sensor can measure electromagnetic energy from electrical potentials traveling along muscles. In another example, neuromuscular activity sensors can be resistive EMG sensors. In an example, an EMG sensor can be a bipolar sensor comprising a ground electrode and a sensor electrode. In another example, neuromuscular activity sensors can comprise electrodes.

In an example, an EMG sensor can comprise a first layer of material with a first level of electroconductivity and a second layer of material with a second level of electroconductivity, wherein the first layer is closer to the surface of the person's body than the second layer, and wherein the second level is greater than the first level. In an example, an EMG sensor can comprise a first layer of material with a first level of permittivity and a second layer of material with a second level of permittivity, wherein the first layer is closer to the surface of the person's body than the second layer, and wherein the second level is greater than the first level. In an example, an EMG sensor can comprise an electroconductive layer which is made from an elastomeric polymer which has been doped, impregnated, and/or coated with aluminum particles. In an example, an EMG sensor can comprise an electroconductive layer which is made from an elastomeric polymer which has been doped, impregnated, and/or coated with conductive material. In an example, an EMG sensor can include a high-permittivity layer and/or coating.

In another example, an EMG sensor can be coated with dielectric material. In an example, an EMG sensor can comprise a dielectric layer which contacts a person's skin and a conductive layer on top of the dielectric layer which does not contact the person's skin. In another example, an EMG sensor can comprise a layer of dielectric material between two layers of electroconductive material. In an example, an EMG sensor can comprise an electroconductive layer and two dielectric layers. In another example, the fabric (e.g. textile) of a sleeve or cuff can function as a dielectric layer for capacitive EMG sensors in the sleeve or cuff. In an example, EMG sensors on a band can have one or more Shore OO values between 20 and 50.

In one embodiment, a band, strap, or sleeve with neuromuscular activity (e.g. EMG) sensors can have EMG sensors with half-cylindrical shapes which protrude out from the inner circumference of the band toward the surface of a person's wrist and/or forearm. In an example, an EMG sensor on a band can have a circular shape. In an example, an EMG sensor on a band can have a hexagonal shape. In an example, an EMG sensor on a band can have an arcuate shape. In an example, neuromuscular activity sensors can have arcuate (e.g. circular) or polygonal (e.g. quadrilateral) cross-sectional shapes. In an example, neuromuscular activity sensors can have longitudinal (e.g. oblong or rectangular) cross-sectional shapes.

In another example, the body-facing surfaces of EMG sensors in a band can concave, wherein the concavities face (e.g. are open toward) the person's body. In an example, EMG sensors around the circumference of a band can have different sizes. In another example, an EMG sensor can be made by printing electroconductive ink. In another example, an EMG sensor can be made by embroidering conductive material onto a low-conductivity textile or fabric. In an example, neuromuscular activity (e.g. EMG) sensors can be formed by selectively weaving, sewing, braiding, or embroidering electroconductive yarns, threads, or fibers into selected areas of the fabric used to create a sleeve before the sleeve is formed.

In one embodiment, a band with neuromuscular activity (e.g. EMG) sensors can comprise a first series of sensors distributed around a first circumferential line of the band and a second series of sensors distributed around a second circumferential line of the band. In an example, EMG sensors can be at different polar coordinate locations around a circumference of a band. In an example, neuromuscular activity (e.g. EMG) sensors can be distributed around a person's wrist (and/or forearm) in at least one circumference ring which intersects the display screen (and/or watch housing) and in also at least one circumferential ring which is at least one inch proximal to the proximal edge of the display screen (and/or watch housing) In an example, neuromuscular activity (e.g. EMG) sensors on a band can be aligned along one or more circumferential rings around the band and/or the person's wrist (and/or forearm). In an example, pairs of EMG sensors around the circumference of a band can be pair-wise coplanar.

In an example, EMG sensors on a band can collectively span between 40% and 60% of the circumference of a wrist or forearm. In another example, at least 30% of EMG sensors on band can be configured to contact the ventral side of a wrist or forearm. In an example, EMG sensors on a band can be disproportionately located (e.g. clustered) on the ventral and lateral surfaces of a wrist or forearm. In another example, EMG sensors on a portion of a band which spans the ventral surface of a person's wrist can be more densely-distributed than EMG sensors on the rest of the band. In an example, EMG sensors on a ventral-facing side of a band can be closer together than EMG sensor on the dorsal-facing side of the band. In another example, the majority of EMG sensors on band can be configured to contact the ventral side and lateral sides of a wrist or forearm.

In an example, EMG sensors can be evenly spaced on the ventral surface and lateral surfaces of a person's wrist or forearm. In one embodiment, a longitudinal axis of a longitudinal neuromuscular activity (e.g. EMG) sensor can intersect a circumference of a band at an angle between 30 and 60 degrees. In an example, a band, strap, or sleeve with modular neuromuscular activity (e.g. EMG) sensors can have a plurality of attachment receptors (e.g. ports or snaps) around its circumference, wherein modular sensors can be removably attached to different attachment receptors at different times. In an example, a modular neuromuscular activity (e.g. EMG) sensor can be removably-attached to a band at different locations at different times by an attachment mechanism selected from the group consisting of: clasp, clip, hook, magnet, pin, plug, port, and snap. In an example, modular neuromuscular activity (e.g. EMG) sensors can be modular.

In an example, different subsets of EMG sensors on a band can be selectively activated at different times based on which activities the person wearing the band is doing at those different times. In another example, modular neuromuscular activity (e.g. EMG) sensors which can be removably-attached to different locations on a band, strap, or sleeve can enable customizing the configuration of sensors to the anatomy of a specific person. In an example, selected sensors on a band can be moved laterally and/or radially in order to customize sensor configuration to the anatomy of a specific person.

In another example, a band, strap, or sleeve with neuromuscular activity (e.g. EMG) sensors can further comprise a circumferential track, channel, or ring around its inner perimeter, wherein the distances between one or more EMG sensors on the band can be adjusted by sliding one or more EMG sensors along the track, channel, or ring. In another example, a band, strap, or sleeve with neuromuscular activity (e.g. EMG) sensors can further comprise a plurality of electromagnetic actuators, wherein the actuators change the radial locations of the EMG sensors on the circumference of the band.

In an example, a band, strap, or sleeve with neuromuscular activity (e.g. EMG) sensors can further comprise a track, channel, or ring around its inner perimeter, wherein the distances between one or more EMG sensors on the band can be adjusted by sliding one or more EMG sensors along the track, channel, or ring. In an example, a band, strap, or sleeve with neuromuscular activity (e.g. EMG) sensors can further comprise one or more circumferential tracks, channels, or rings around its inner perimeter, wherein one or more EMG sensors are moved (e.g. slid) along the one or more tracks, channels, or rings by one or more electromagnetic actuators. In an example, a band, strap, or sleeve with neuromuscular activity (e.g. EMG) sensors can further comprise two parallel tracks, channels, or rings around its inner perimeter, wherein one or more EMG sensors can be moved to different radial locations by sliding along the tracks, channels, or rings.

In one embodiment, a circumferential channel or track on a band can span some (or all) of the circumference of the band. In an example, an EMG sensor can be moved along a track, channel, or ring around a portion of the inner perimeter of a band which is worn around a person's wrist and/or forearm. In an example, device can further comprise an electromagnetic actuator which automatically moves one or more neuromuscular activity (e.g. EMG) sensors along a circumferential channel, track, or ring. In an example, distances between sensors on a wearable band can be selectively adjusted by moving them along one or more channels, tracks, or rings on the band in order to more-accurately record the activity of specific muscles. In another example, the radial location of one or more EMG sensors on a person's wrist can be adjusted. In an example, the radial locations of EMG sensors on a band can be adjusted by one or more electromagnetic actuators.

In another example, a channel or track along which an EMG sensor slides can be orthogonal to a circumference of the band. In an example, a neuromuscular activity (e.g. EMG) sensor can be moved laterally across (a portion of) a person's wrist (and/or forearm) by moving (e.g. sliding) the sensor along a lateral track or pathway. In another example, an EMG sensor on a band can be automatically moved across a portion of the width of the band by an electromagnetic actuator (e.g. miniature electric motor). In an example, device can further comprise an electromagnetic actuator which automatically moves one or more neuromuscular activity (e.g. EMG) sensors along a lateral channel, track, or ring.

In an example, a band, strap, or sleeve with neuromuscular activity (e.g. EMG) sensors can further comprise a plurality of electromagnetic actuators. wherein a selected subset of EMG sensors can be moved by activating a selected subset of actuators (e.g. to change the distance by which the EMG sensors protrude out from the body-facing surface of the band). In an example, a selected set of one or more neuromuscular activity (e.g. EMG) sensors can have a first configuration in which they protrude a first distance from the band and second configuration in which they protrude a second distance from the band, wherein the second distance is greater than the first distance.

In one embodiment, adjacent EMG sensors around the circumference of a band can be separated by different distances, wherein EMG sensors on a portion of the band which spans the ventral surface of a person's wrist are closer together than EMG sensors on the rest of the band. In an example, distances between two or more EMG sensors in a band can be adjusted. In an example, the distances by which EMG sensors protrude out from a band can be selectively and independently adjusted by selective activation of electromagnetic actuators on the band. In another example, when signals from one or more EMG sensors on a band are weak or inaccurate, then distances between those EMG sensors and the person's body can be automatically adjusted by one or more electromagnetic actuators.

In an example, the amount of pressure exerted by one or more EMG sensors on a person's wrist can be automatically adjusted by an electromagnetic actuator in response to lack of direct contact between the sensor and the person's wrist. In another example, the proximity and/or force of one or more EMG sensors relative to a wrist or forearm can be changed by one or more pneumatic mechanisms. In another example, a wearable device (e.g. a band, smart watch, or watch band) can have selectively-rotatable neuromuscular activity (e.g. EMG) sensors. In an example, different subsets of EMG sensors on a band can be selectively activated at different times. In an example, an actuator on a band can be a brushless DC motor. In an example, an actuator on a band can be a MEMS device.

In one embodiment, a band, strap, or sleeve with neuromuscular activity (e.g. EMG) sensors can include one or more solenoids which change the distances by which one or more sensors protrude out from the band toward the wrist. In an example, the amount of pressure exerted by one or more EMG sensors on a person's wrist can be adjusted by an electromagnetic solenoid. In an example, the pressure between a neuromuscular activity (e.g. EMG) sensor on a band and a person's wrist (and/or forearm) can be adjusted by activation of a piston or solenoid between the sensor and the band.

In another example, a band, strap, or sleeve can have two expandable (e.g. elastic and/or stretchable) sections which connect to sides of a display screen (and/or watch housing). In an example, a wearable device (e.g. a band, smart watch, or watch band) with neuromuscular activity (e.g. EMG) sensors can comprise: a band, strap, or sleeve which is configured to be worn around at least 50% of a circumference of a person's wrist (and/or forearm); a plurality of neuromuscular activity (e.g. EMG) sensors on the band, wherein there are (expandable) fluid-filled chambers within the sensors. In another example, the distance between a neuromuscular activity (e.g. EMG) sensor on a band and a person's wrist (and/or forearm) can be adjusted by expansion or shrinkage of an expandable chamber between the sensor and the band. In an example, the pressure between a neuromuscular activity (e.g. EMG) sensor and a person's wrist (and/or forearm) can be adjusted by expansion or shrinkage of an expandable chamber between the sensor and the band.

In an example, a band or strap with neuromuscular activity (e.g. EMG) sensors can comprise a plurality of inflatable sections. In an example, the amount of pressure exerted by one or more EMG sensors on a person's wrist can be adjusted by inflation of an inflatable chamber. In an example, the length (and fit) of a band or strap with neuromuscular activity (e.g. EMG) sensors can be adjusted via a plurality of inflatable sections around the circumference of the band or strap. In another example, the radial location of one or more EMG sensors on a person's wrist can be adjusted by inflation of an inflatable chamber.

In one embodiment, a band with neuromuscular activity (e.g. EMG) sensors can comprise one or more adjustable-length piezoelectric sections. In an example, a stretchable section of a band can be piezoelectric. In an example, stretchable sections of a band can be made with piezoelectric material, wherein the stretchability and/or lengths of the stretchable sections can be adjusted by transmission of electrical energy through them. In an example, the radial location of one or more EMG sensors on a person's wrist can be adjusted by a piezoelectric actuator. In another example, the amount of tension in springs, pistons, or other tensile components in a band can be adjusted in order to change the amount of force required to expand the band. In an example, the size and/or tension of the circumference of a band can be adjusted by changing the tension of a spring.

In an example, a band or strap with neuromuscular activity (e.g. EMG) sensors can comprise one or more telescoping sections, wherein components of a telescoping section are connected by springs, pistons, or other tensile components so that they are held in tension and force is required to change the extent to which one component is inserted into another component. In one embodiment, a band, strap, or sleeve with neuromuscular activity (e.g. EMG) sensors can function as a watch strap with an alternating series of electronic housings (wherein each housing contains one or more EMG sensors) and telescoping components (which are compelled apart by tension from internal springs), wherein the series alternates (back and forth) between electronic housings and telescoping components along the strap. In an example, the length (and fit) of a band or strap with neuromuscular activity (e.g. EMG) sensors can be changed by adjusting one or more telescoping sections around the circumference of the band or strap.

In another example, a band, strap, or sleeve with neuromuscular activity (e.g. EMG) sensors can further comprise a data processor. In an example, a band, strap, or sleeve with neuromuscular activity (e.g. EMG) sensors can include an amplification circuit. In an example, an EMG sensor can further comprise a signal amplifier. In an example, a band, strap, or sleeve with neuromuscular activity (e.g. EMG) sensors can further comprise one or more batteries. In one embodiment, a band, strap, or sleeve with neuromuscular activity (e.g. EMG) sensors can further comprise a (built-in) display screen. In an example, a band, strap, or sleeve with neuromuscular activity (e.g. EMG) sensors can hold a quadrilateral display screen and/or watch housing on a person's wrist. In an example, a wearable computing device for the wrist and/or arm can comprise a folding display screen. In an example, display screen (and/or watch housing) can be an integral part of a device.

In one embodiment, an EMG sensor can be made from gold, steel, stainless steel, silver, titanium, electrically conductive rubber, and/or electrically conductive silicone. In an example, an EMG sensor can be made from thermoplastic urethane which has been doped, impregnated, and/or coating with silver and carbon nanotubes. In an example, an EMG sensor can be made with an elastomeric polymer (e.g. PDMS) which has been doped, impregnated, and/or coated with electroconductive material (e.g. carbon, silver, or aluminum). In an example, an EMG sensor can have a low-conductivity (or non-conductive) coating made from acetate, acrylic, cotton, denim, elastane, latex, linen, Lycra, neoprene, nylon, nylon, polyester, wool, silicone, polydimethylsiloxane (PDMS), silk, spandex, or rayon. In an example, an EMG sensor can include a layer made from an elastomeric polymer. In an example, an EMG sensor can be made from electroconductive threads, fibers, yarns, strands, filaments, traces, and/or layers.

In one embodiment, an EMG sensor can comprise a woven array of electroconductive yarns, threads, or filaments. In an example, a band, strap, or sleeve with neuromuscular activity (e.g. EMG) sensors can further comprise one or more inertial motion sensors. In an example, a band, strap, or sleeve with neuromuscular activity (e.g. EMG) sensors can further comprises a plurality of bending-based (e.g. strain-based) motion sensors. In an example, data from EMG sensors and inertial motion sensors can be jointly analyzed using an Artificial Neural Network (ANN) or other form of machine learning. In an example, a band, strap, or sleeve with neuromuscular activity (e.g. EMG) sensors can further comprise a camera. In an example, a band, strap, or sleeve with neuromuscular activity (e.g. EMG) sensors can further comprise an oxygenation level sensor.

FIG. 1 shows two views of a wearable device (e.g. a band, smart watch, or watch band) with neuromuscular activity (e.g. EMG) sensors comprising: a band (or strap) 103 which is configured to be worn around at least 50% of a circumference of a person's wrist (and/or forearm) 101; a plurality of neuromuscular activity (e.g. EMG) sensors (including sensor 104) on the band; and a display screen (and/or watch housing) 102 which is held on the wrist (and/or forearm) by the band, wherein a maximum (proximal-to-distal) width of the band (or strap) is at least 20% greater than a maximum (proximal-to-distal) width of the display screen (and/or watch housing). In an example, the device can further comprise a battery, an amplifier, a data processor, and/or a data transceiver. The upper portion of FIG. 1 shows a dorsal (e.g. top-down) view of the device and the lower portion of FIG. 1 shows a lateral (e.g. side) view of the device. In this example, proximal means closer to a person's shoulder and distal means farther from the person's shoulder.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, a wearable device (e.g. a band, smart watch, or watch band) with neuromuscular activity (e.g. EMG) sensors can comprise: a band (or strap) which is configured to be worn around at least 50% of a circumference of a person's wrist (or forearm); and a plurality of neuromuscular activity (e.g. EMG) sensors on the band. In an example, a maximum (proximal-to-distal) width of the band (or strap) is at least 20% greater than a maximum (proximal-to-distal) width of a smart watch housing. In an example, a watch housing can be removably attached to the band (or strap). In an example, the sensors can be in wireless communication with electronics in the watch housing.

In an example, a maximum (proximal-to-distal) width of a band (or strap) can be at least 50% greater than a maximum (proximal-to-distal) width of a display screen (and/or watch housing). In an example, an average (proximal-to-distal) width of a band (or strap) can be at least 20% greater than an average (proximal-to-distal) width of a display screen (and/or watch housing). In an example, the average width of a ventral portion of a band can be at least 20% greater than the average width of a dorsal portion of the band.

In an example, a band (or strap) can comprise a continuous piece of flexible polymer material. In an example, a band (or strap) can comprise a series of flexibly-connected rigid components (e.g. links or housings). In an example, a band (or strap) can be made from a breathable fabric and/or textile. In an example, a band (or strap) can comprise an alternating series of rigid components and flexible (e.g. elastic) components (e.g. alternating between rigid and flexible components) around (at least a portion of) the circumference of a person's wrist (and/or forearm). In an example, flexible components in this series can further comprise flexible electroconductive pathways. Relevant variations discussed elsewhere in this disclosure or priority-linked disclosures can also be applied to this example.

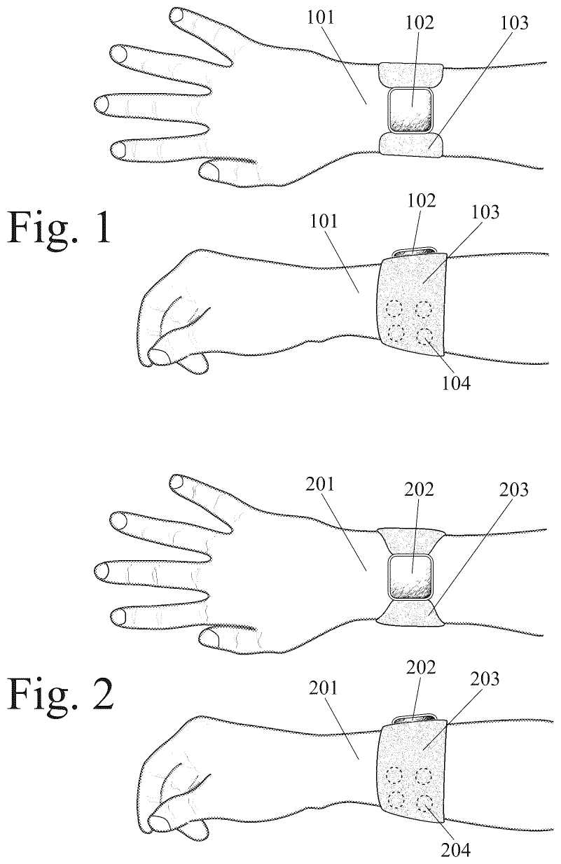

FIG. 2 shows a wearable device (e.g. a band, smart watch, or watch band) with neuromuscular activity (e.g. EMG) sensors which is like the one shown in FIG. 1 except that the width of the band ends taper toward where they connect to the display screen (and/or watch housing).

Specifically, FIG. 2 shows two views of a wearable device (e.g. a band, smart watch, or watch band) with neuromuscular activity (e.g. EMG) sensors comprising: a band (or strap) 203 which is configured to be worn around at least 50% of a circumference of a person's wrist (and/or forearm) 201; a plurality of neuromuscular activity (e.g. EMG) sensors (including sensor 204) on the band; and a display screen (and/or watch housing) 202 which is held on the wrist (and/or forearm) by the band, wherein the width of the ends of the band taper toward where they connect to the display screen (and/or watch housing). In an example, the device can further comprise a battery, an amplifier, a data processor, and/or a data transceiver. The upper portion of FIG. 2 shows a dorsal (e.g. top-down) view of the device and the lower portion of FIG. 2 shows a lateral (e.g. side) view of the device. In this example, proximal means closer to a person's shoulder and distal means farther from the person's shoulder.

In this example, the widths of the ends of the band on the dorsal surface of a person's wrist (and/or forearm) taper as they approach connection with the display screen (and/or watch housing). In this example, the widths of the ends of the band to which the display screen (and/or watch housing) are attached are the same as the width of the display screen (and/or watch housing), but the average width of the rest of the band is greater than the width of the display. In this example, the combined shape of the dorsal ends of the band and the display screen (and/or watch display) is substantially convex. In other words, this combined shape is convex overall, with a relatively-straight central section formed by the proximal and distal sides of the display screen (and/or watch display). Relevant variations discussed elsewhere in this disclosure or priority-linked disclosures can also be applied to this example.

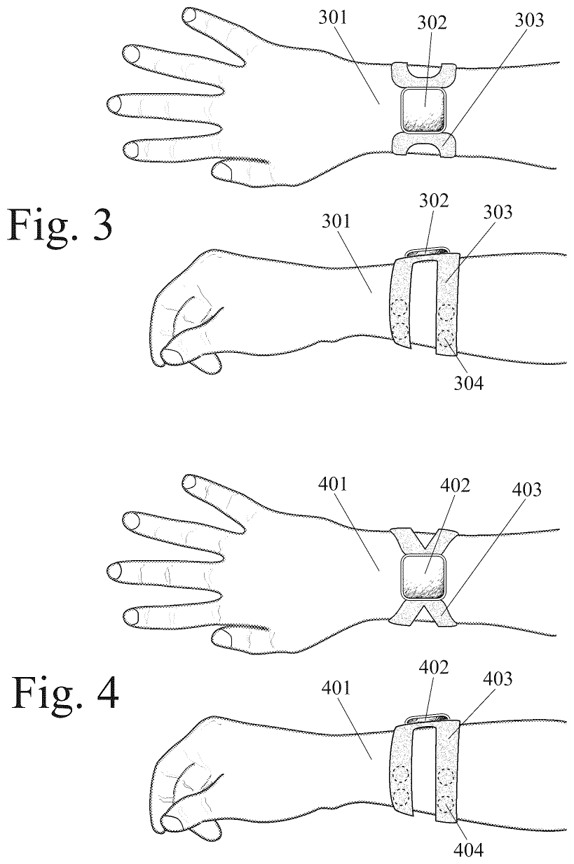

FIG. 3 shows two views of a wearable device (e.g. a band, smart watch, or watch band) with neuromuscular activity (e.g. EMG) sensors comprising: a bifurcating band (or strap) 303 which is configured to be worn around at least 50% of a circumference of a person's wrist (and/or forearm) 301; a plurality of neuromuscular activity (e.g. EMG) sensors (including sensor 304) on the band; and a display screen (and/or watch housing) 302 which is held on the wrist (and/or forearm) by the band, wherein the band bifurcates on a dorsal surface of the person's wrist (and/or forearm). In an example, the device can further comprise a battery, an amplifier, a data processor, and/or a data transceiver. The upper portion of FIG. 3 shows a dorsal (e.g. top-down) view of the device and the lower portion of FIG. 3 shows a lateral (e.g. side) view of the device.

In this example, the widths of ends of a bifurcating band are greater than the width of a display screen (and/or watch housing) where these ends connect to the display screen (and/or watch housing). In an example, a bifurcating band can bifurcate into two bands on a dorsal surface of a person's wrist (and/or forearm), forming two (parallel) branches which span the ventral surface of the wrist (and/or forearm). In an example, a bifurcating band can bifurcate into two bands on the dorsal surface of a person's wrist (and/or forearm), forming two (parallel) branches which span the lateral and ventral surfaces of the wrist (and/or forearm). In an example, the distance between two branches on the ventral surface of the wrist (and/or forearm) can be greater than 75% of the width of a display screen (and/or watch housing). In an example, the distance between two branches on the ventral surface of the wrist (and/or forearm) can be less than 125% of the width of a display screen (and/or watch housing). In an example, the distance between two branches on the ventral surface of the wrist (and/or forearm) can be greater than 75% and less than 125% of the width of a display screen (and/or watch housing).

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have arcuate (e.g. circular) or polygonal (e.g. quadrilateral) cross-sectional shapes. In an example, neuromuscular activity sensors can have hemispherical or half-cylindrical shapes.

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, a band (or strap) can comprise a continuous piece of flexible polymer material. In an example, a band (or strap) can comprise a series of flexibly-connected rigid components (e.g. links or housings). In an example, a band (or strap) can be made from a breathable fabric and/or textile. In an example, a band (or strap) can comprise an alternating series of rigid components and flexible (e.g. elastic) components (e.g. alternating between rigid and flexible components) around (at least a portion of) the circumference of a person's wrist (and/or forearm). In an example, flexible components in this series can further comprise flexible electroconductive pathways. Relevant variations discussed elsewhere in this disclosure or priority-linked disclosures can also be applied to this example.

FIG. 4 shows a wearable device (e.g. a band, smart watch, or watch band) with neuromuscular activity (e.g. EMG) sensors which is like the one shown in FIG. 3, except that branches of the bifurcating band split and/or converge at an acute angle on the dorsal surface of the person's wrist (and/or forearm) where they connect to a display screen (and/or watch housing).

FIG. 4 shows two views of a wearable device (e.g. a band, smart watch, or watch band) with neuromuscular activity (e.g. EMG) sensors comprising: a bifurcating band (or strap) 403 which is configured to be worn around at least 50% of a circumference of a person's wrist (and/or forearm) 401, wherein branches of the bifurcating band split and/or converge at an acute angle on the dorsal surface of the person's wrist (and/or forearm); a plurality of neuromuscular activity (e.g. EMG) sensors (including sensor 404) on the band; and a display screen (and/or watch housing) 402 which is held on the wrist (and/or forearm) by the band. In an example, the device can further comprise a battery, an amplifier, a data processor, and/or a data transceiver. The upper portion of FIG. 4 shows a dorsal (e.g. top-down) view of the device and the lower portion of FIG. 4 shows a lateral (e.g. side) view of the device. Relevant variations discussed elsewhere in this disclosure or priority-linked disclosures can also be applied to this example.

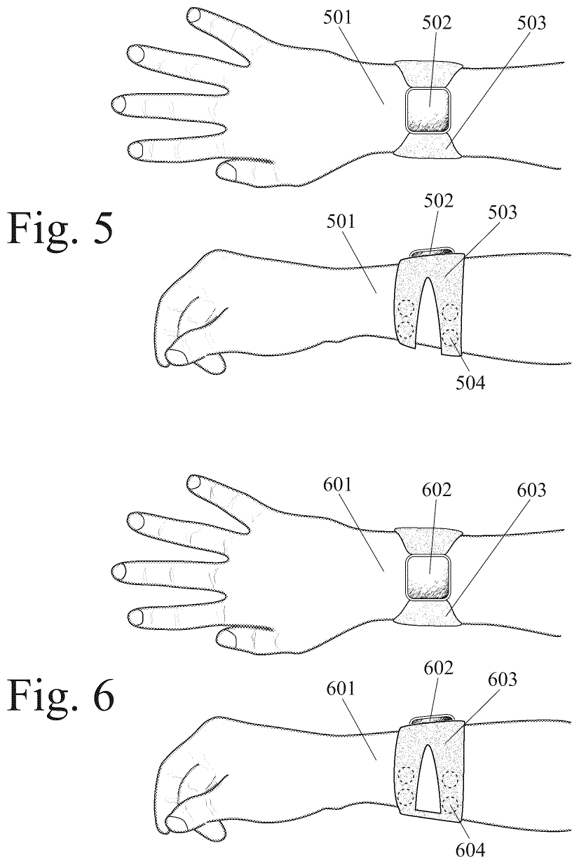

FIG. 5 shows a wearable device (e.g. a band, smart watch, or watch band) with neuromuscular activity (e.g. EMG) sensors which is like the one shown in FIG. 3, except that branches of the bifurcating band split and/or converge on lateral surfaces of the person's wrist (and/or forearm).

FIG. 5 shows two views of a wearable device (e.g. a band, smart watch, or watch band) with neuromuscular activity (e.g. EMG) sensors comprising: a bifurcating band (or strap) 503 which is configured to be worn around at least 50% of a circumference of a person's wrist (and/or forearm) 501, wherein branches of the bifurcating band split and/or converge on the lateral surfaces of the person's wrist (and/or forearm); a plurality of neuromuscular activity (e.g. EMG) sensors (including sensor 504) on the band; and a display screen (and/or watch housing) 502 which is held on the wrist (and/or forearm) by the band. In an example, the device can further comprise a battery, an amplifier, a data processor, and/or a data transceiver. The upper portion of FIG. 5 shows a dorsal (e.g. top-down) view of the device and the lower portion of FIG. 5 shows a lateral (e.g. side) view of the device. Relevant variations discussed elsewhere in this disclosure or priority-linked disclosures can also be applied to this example.

FIG. 6 shows a wearable device (e.g. a band, smart watch, or watch band) with neuromuscular activity (e.g. EMG) sensors which is like the one shown in FIG. 3, except that branches of the bifurcating band split and/or converge on lateral surfaces of the person's wrist (and/or forearm) and there is a connecting strip between the branches on the ventral surface of the person's wrist.

FIG. 6 shows two views of a wearable device (e.g. a band, smart watch, or watch band) with neuromuscular activity (e.g. EMG) sensors comprising: a bifurcating band (or strap) 603 which is configured to be worn around at least 50% of a circumference of a person's wrist (and/or forearm) 601, wherein branches of the bifurcating band split and/or converge on the lateral surfaces of the person's wrist (and/or forearm), and wherein there is a connecting strip between the branches on a ventral surface of the person's wrist (and/or forearm); a plurality of neuromuscular activity (e.g. EMG) sensors (including sensor 604) on the band; and a display screen (and/or watch housing) 602 which is held on the wrist (and/or forearm) by the band. In an example, the device can further comprise a battery, an amplifier, a data processor, and/or a data transceiver. The upper portion of FIG. 6 shows a dorsal (e.g. top-down) view of the device and the lower portion of FIG. 6 shows a lateral (e.g. side) view of the device. Relevant variations discussed elsewhere in this disclosure or priority-linked disclosures can also be applied to this example.

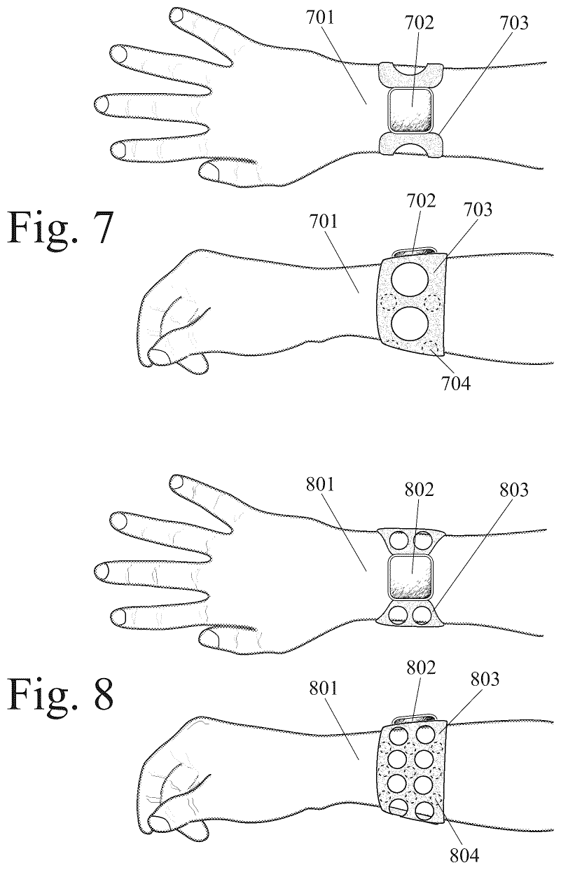

FIG. 7 shows two views of a wearable device (e.g. a band, smart watch, or watch band) with neuromuscular activity (e.g. EMG) sensors comprising: a band (or strap) 703 which is configured to be worn around at least 50% of a circumference of a person's wrist (and/or forearm) 701, wherein there is a (partial) circumferential array (e.g. a ring) of holes around the (partial) circumference of the band; a plurality of neuromuscular activity (e.g. EMG) sensors (including sensor 704) on the band; and a display screen (and/or watch housing) 702 which is held on the wrist (and/or forearm) by the band. In an example, the device can further comprise a battery, an amplifier, a data processor, and/or a data transceiver. The upper portion of FIG. 7 shows a dorsal (e.g. top-down) view of the device and the lower portion of FIG. 7 shows a lateral (e.g. side) view of the device.

In an example, a holes in a band (or strap) can be arcuate. In an example, a hole in a band (or strap) can be circular, oval, or elliptical. In an example, a holes in a band (or strap) can be polygonal (e.g. hexagonal) in shape. In an example, a hole in a band (or strap) can span between 25% and 75% of the (proximal-to-distal) width of the band. In an example, there can be a single (partial) circumferential array (e.g. single ring) of holes around the (partial) circumference of a band (or strap). In an example, a single circumferential array can span the center of the width of the band. In an example, there can be two (partial) circumferential arrays (e.g. two rings) of holes around the (partial) circumference of a band (or strap), wherein the planes of the arrays are parallel to each other. In an example, there can be a three or more (partial) circumferential arrays (e.g. three rings) of holes around the (partial) circumference of a band (or strap). In an example, there can be four holes around a (partial) circumference of a band (or strap). In an example, this device can comprise a holy band (e.g. I Chronicles 25). In an example, there can be 2 to 6 holes around a (partial) circumference of a band (or strap).

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes.

In an example, neuromuscular activity (e.g. EMG) sensors can be located between holes on a band (or strap). In an example, neuromuscular activity (e.g. EMG) sensors can be located at radial locations between holes on a band (or strap). In an example, neuromuscular activity (e.g. EMG) sensors can be located proximally and distally relative to holes on a band (or strap). In an example, pairs of electrodes can be located proximally and distally relative to holes on a band (or strap). In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, a band (or strap) can comprise a continuous piece of flexible polymer material. In an example, a band (or strap) can comprise a series of flexibly-connected rigid components (e.g. links or housings). In an example, a band (or strap) can be made from a breathable fabric and/or textile. In an example, a band (or strap) can comprise an alternating series of rigid components and flexible (e.g. elastic) components (e.g. alternating between rigid and flexible components) around (at least a portion of) the circumference of a person's wrist (and/or forearm). In an example, flexible components in this series can further comprise flexible electroconductive pathways. Relevant variations discussed elsewhere in this disclosure or priority-linked disclosures can also be applied to this example.

FIG. 8 shows a wearable device (e.g. a band, smart watch, or watch band) with neuromuscular activity (e.g. EMG) sensors which is like the one shown in FIG. 7, except that there are two parallel (partial) circumferential arrays (e.g. rings) of holes around the (partial) circumference of the band.

FIG. 8 shows two views of a wearable device (e.g. a band, smart watch, or watch band) with neuromuscular activity (e.g. EMG) sensors comprising: a band (or strap) 803 which is configured to be worn around at least 50% of a circumference of a person's wrist (and/or forearm) 801, wherein there are two parallel (partial) circumferential arrays (e.g. rings) of holes around the (partial) circumference of the band; a plurality of neuromuscular activity (e.g. EMG) sensors (including sensor 804) on the band; and a display screen (and/or watch housing) 802 which is held on the wrist (and/or forearm) by the band. In an example, the device can further comprise a battery, an amplifier, a data processor, and/or a data transceiver. The upper portion of FIG. 8 shows a dorsal (e.g. top-down) view of the device and the lower portion of FIG. 8 shows a lateral (e.g. side) view of the device. Relevant variations discussed elsewhere in this disclosure or priority-linked disclosures can also be applied to this example.

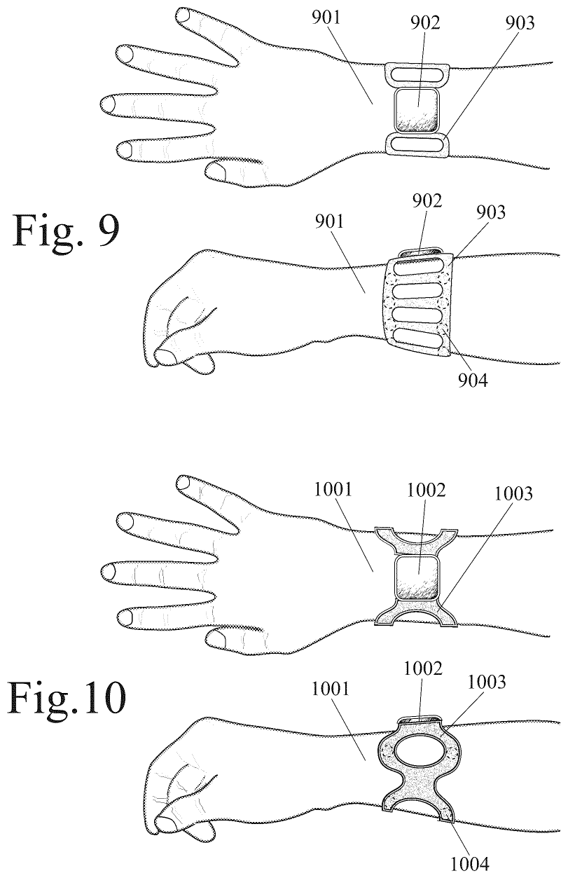

FIG. 9 shows a wearable device (e.g. a band, smart watch, or watch band) with neuromuscular activity (e.g. EMG) sensors which is like the one shown in FIG. 7, except that holes in the band have longitudinal axes which are substantially orthogonal to the (partial) circumference of the band.

FIG. 9 shows two views of a wearable device (e.g. a band, smart watch, or watch band) with neuromuscular activity (e.g. EMG) sensors comprising: a band (or strap) 903 which is configured to be worn around at least 50% of a circumference of a person's wrist (and/or forearm) 901, wherein there are a plurality of holes in the band with longitudinal axes which are substantially orthogonal to the (partial) circumference of the band; a plurality of neuromuscular activity (e.g. EMG) sensors (including sensor 904) on the band; and a display screen (and/or watch housing) 902 which is held on the wrist (and/or forearm) by the band. In an example, the device can further comprise a battery, an amplifier, a data processor, and/or a data transceiver. The upper portion of FIG. 9 shows a dorsal (e.g. top-down) view of the device and the lower portion of FIG. 9 shows a lateral (e.g. side) view of the device. Relevant variations discussed elsewhere in this disclosure or priority-linked disclosures can also be applied to this example.

FIG. 10 shows a wearable device (e.g. a band, smart watch, or watch band) with neuromuscular activity (e.g. EMG) sensors which is like the one shown in FIG. 7, except that the proximal and distal sides of the band are undulating (e.g. sinusoidal).

FIG. 10 shows two views of a wearable device (e.g. a band, smart watch, or watch band) with neuromuscular activity (e.g. EMG) sensors comprising: a band (or strap) 1003 which is configured to be worn around at least 50% of a circumference of a person's wrist (and/or forearm) 1001, wherein there are a plurality of holes in the band, and wherein the proximal and distal sides of the band are undulating (e.g. sinusoidal); a plurality of neuromuscular activity (e.g. EMG) sensors (including sensor 1004) on the band; and a display screen (and/or watch housing) 1002 which is held on the wrist (and/or forearm) by the band. In an example, the device can further comprise a battery, an amplifier, a data processor, and/or a data transceiver. The upper portion of FIG. 10 shows a dorsal (e.g. top-down) view of the device and the lower portion of FIG. 10 shows a lateral (e.g. side) view of the device.

In an example, undulations of the proximal and distal sides of the band can be symmetric with respect to a central circumference of the band. In an example, there can be one hole for each full phase undulation (e.g. two-phase sinusoidal wave) of the band. In an example, the band can have between 2 and 6 undulations. In an example, the band can have more than six undulations. Relevant variations discussed elsewhere in this disclosure or priority-linked disclosures can also be applied to this example.

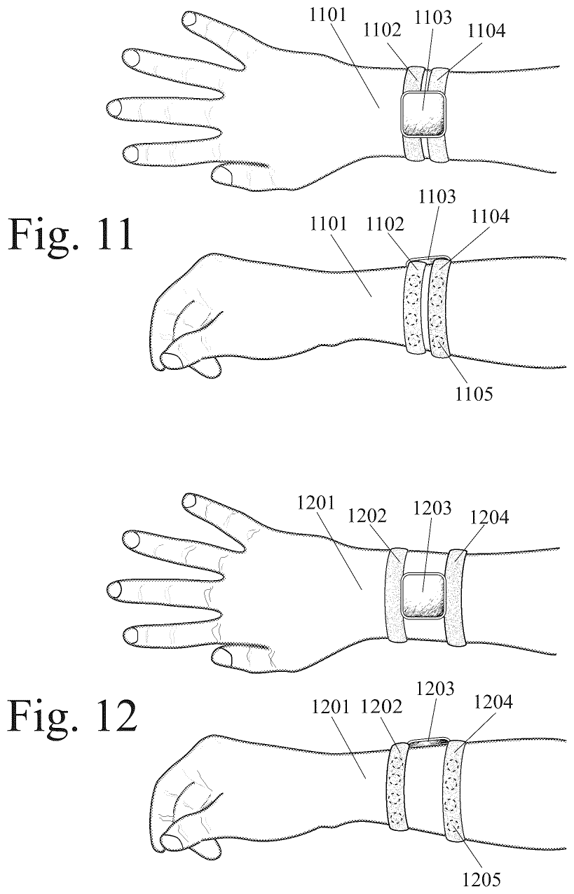

FIG. 11 shows two views of a wearable device (e.g. a band, smart watch, or watch band) with neuromuscular activity (e.g. EMG) sensors comprising: a dual band (or strap) which is configured to be worn around at least 50% of a circumference of a person's wrist (and/or forearm) 1101, wherein the dual band further comprises a proximal band 1104 and a distal band 1102; a plurality of neuromuscular activity (e.g. EMG) sensors (including sensor 1105) on the dual band; and a display screen (and/or watch housing) 1103 which is held on the wrist (and/or forearm) by the dual band. In an example, the device can further comprise a battery, an amplifier, a data processor, and/or a data transceiver. The upper portion of FIG. 11 shows a dorsal (e.g. top-down) view of the device and the lower portion of FIG. 11 shows a lateral (e.g. side) view of the device. In this example, proximal means closer to a person's shoulder and distal means farther from the person's shoulder.

In an example, the proximal and distal bands can be substantially parallel to each other. In an example, the proximal and distal bands can be the same width. In an example, the combined widths of the proximal and distal bands can be less than the width of the display screen (and/or watch housing). In an example, there can be a gap between the proximal and distal bands. In an example, the combined widths of the proximal band, the gap, and the distal band can be the same as the width of the display screen (and/or watch housing). In an example, the size of the gap between the proximal and distal bands can be adjusted. In an example, the combined widths of the proximal band, the gap, and the distal band can be greater than the width of the display screen (and/or watch housing). In an example, there can be neuromuscular activity (EMG) sensors on both the proximal and distal bands.

In an example, proximal and distal bands can each comprise a continuous piece of flexible polymer material. In an example, proximal and distal bands can each comprise a series of flexibly-connected rigid components (e.g. links or housings). In an example, proximal and distal bands can be made from a breathable fabric and/or textile. In an example, proximal and distal bands can comprise an alternating series of rigid components and flexible (e.g. elastic) components (e.g. alternating between rigid and flexible components) around (at least a portion of) the circumference of a person's wrist (and/or forearm). In an example, flexible components in a series can further comprise flexible electroconductive pathways.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm). Relevant variations discussed elsewhere in this disclosure or priority-linked disclosures can also be applied to this example.

FIG. 12 shows a wearable device (e.g. a band, smart watch, or watch band) with neuromuscular activity (e.g. EMG) sensors which is like the one shown in FIG. 11, except that a gap between proximal and distal bands is larger.

FIG. 12 shows two views of a wearable device (e.g. a band, smart watch, or watch band) with neuromuscular activity (e.g. EMG) sensors comprising: a dual band (or strap) which is configured to be worn around at least 50% of a circumference of a person's wrist (and/or forearm) 1201, wherein the dual band further comprises a proximal band 1204 and a distal band 1202; a plurality of neuromuscular activity (e.g. EMG) sensors (including sensor 1205) on the dual band; and a display screen (and/or watch housing) 1203 which is held on the wrist (and/or forearm) by the dual band. In an example, the device can further comprise a battery, an amplifier, a data processor, and/or a data transceiver. The upper portion of FIG. 12 shows a dorsal (e.g. top-down) view of the device and the lower portion of FIG. 12 shows a lateral (e.g. side) view of the device. In this example, proximal means closer to a person's shoulder and distal means farther from the person's shoulder.

In an example, the gap between proximal and distal bands can be wider than either the proximal band or the distal band. In an example, the gap between proximal and distal bands can be equal to the width of the display screen (and/or watch housing). In an example, the proximal and distal sides of the display screen (and/or watch) band can be removably connected to the proximal and distal bands, respectively. Relevant variations discussed elsewhere in this disclosure or priority-linked disclosures can also be applied to this example.

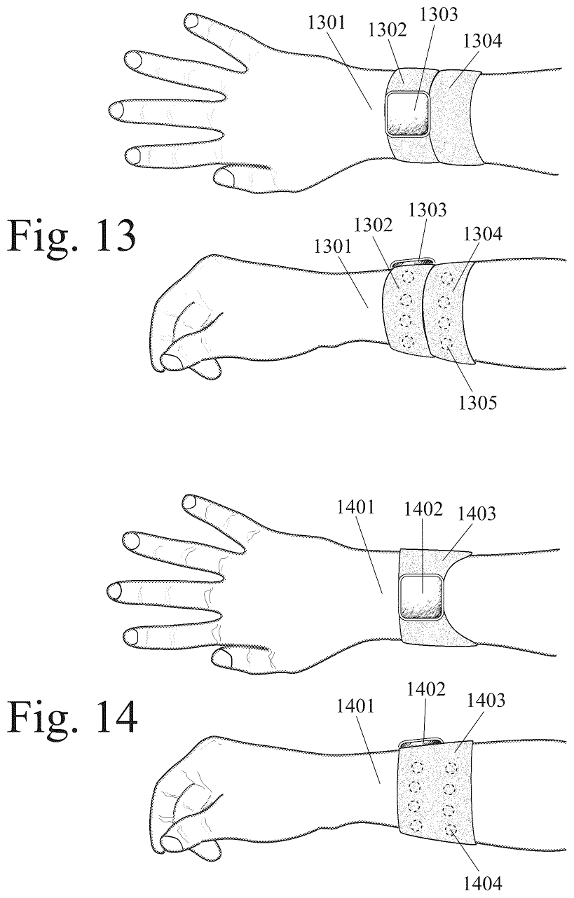

FIG. 13 shows a wearable device (e.g. a band, smart watch, or watch band) with neuromuscular activity (e.g. EMG) sensors which is like the one shown in FIG. 11, except that the display screen (and/or watch housing) is only attached to the distal band. Also, in this example there is no gap between the proximal and distal bands.

FIG. 13 shows two views of a wearable device (e.g. a band, smart watch, or watch band) with neuromuscular activity (e.g. EMG) sensors comprising: a dual band (or strap) which is configured to be worn around at least 50% of a circumference of a person's wrist (and/or forearm) 1301, wherein the dual band further comprises a proximal band 1304 and a distal band 1302; a plurality of neuromuscular activity (e.g. EMG) sensors (including sensor 1305) on the dual band; and a display screen (and/or watch housing) 1303 which is held on the wrist (and/or forearm) by the dual band. In an example, the device can further comprise a battery, an amplifier, a data processor, and/or a data transceiver. The upper portion of FIG. 13 shows a dorsal (e.g. top-down) view of the device and the lower portion of FIG. 13 shows a lateral (e.g. side) view of the device. In this example, proximal means closer to a person's shoulder and distal means farther from the person's shoulder.

In this example, the display screen (and/or watch housing) is only attached to the distal band. In this example, there is no gap between the proximal and distal bands. In another example, there can be a gap between the proximal and distal bands. In an example, one or both of the proximal and distal bands can be rotated relative to the other band. In an example, the bands are non-movably connected to each other and neither band rotates. In an example, the proximal band can be reversibly slid under (or out from under) the distal band. In an example, the proximal band can be reversibly telescoped into (or out from) the distal band. In an example, the proximal and distal bands can be the same width. In another example, the proximal band can be wider than the distal band. Relevant variations discussed elsewhere in this disclosure or priority-linked disclosures can also be applied to this example.

FIG. 14 shows two views of a wearable device (e.g. a band, smart watch, or watch band) with neuromuscular activity (e.g. EMG) sensors comprising: an asymmetric band (or strap) 1403 which is configured to be worn around at least 50% of a circumference of a person's wrist (and/or forearm) 1401, wherein the distal edge or the band is asymmetric relative to the proximal edge of the band; a plurality of neuromuscular activity (e.g. EMG) sensors (including sensor 1404) on the band; and a display screen (and/or watch housing) 1402 which is held on the wrist (and/or forearm) by the band. In an example, the device can further comprise a battery, an amplifier, a data processor, and/or a data transceiver. The upper portion of FIG. 14 shows a dorsal (e.g. top-down) view of the device and the lower portion of FIG. 14 shows a lateral (e.g. side) view of the device. In this example, proximal means closer to a person's shoulder and distal means farther from the person's shoulder.

In an example, the distal edge of a band (or strap) can be asymmetric with respect to the proximal edge of the band. In an example, the distal edge of the band can be coplanar (e.g. a circle or oval in a single plane), but the proximal edge of the band can be arcuate (spanning multiple planes). In an example, the proximal edge of the band can be proximally-concave, wherein the concavity opens in a proximal direction. In an example, the proximal edge of the band can curve (in a proximal direction away from where it connects to a display screen (and/or watch housing). In an example, the width of a portion of the band which spans the ventral surface of a person's wrist (and/or forearm) can be at least 20% greater than the width of a portion of the band which spans the dorsal surface of the person's wrist because the proximal edge of the band is proximally-concave.

In an example, a band (or strap) can comprise a continuous piece of flexible polymer material. In an example, a band (or strap) can comprise a series of flexibly-connected rigid components (e.g. links or housings). In an example, a band (or strap) can be made from a breathable fabric and/or textile. In an example, a band (or strap) can comprise an alternating series of rigid components and flexible (e.g. elastic) components (e.g. alternating between rigid and flexible components) around (at least a portion of) the circumference of a person's wrist (and/or forearm). In an example, flexible components in this series can further comprise flexible electroconductive pathways.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array. Relevant variations discussed elsewhere in this disclosure or priority-linked disclosures can also be applied to this example.

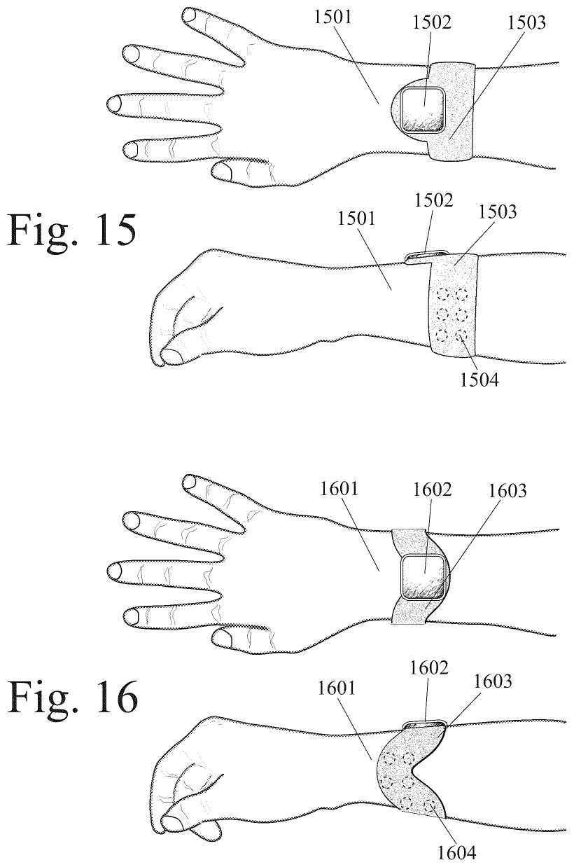

FIG. 15 shows two views of a wearable device (e.g. a band, smart watch, or watch band) with neuromuscular activity (e.g. EMG) sensors comprising: an asymmetric band (or strap) 1503 which is configured to be worn around at least 50% of a circumference of a person's wrist (and/or forearm) 1501, wherein the distal edge or the band is asymmetric relative to the proximal edge of the band; a plurality of neuromuscular activity (e.g. EMG) sensors (including sensor 1504) on the band; and a display screen (and/or watch housing) 1502 which is held on the wrist (and/or forearm) by the band. In an example, the device can further comprise a battery, an amplifier, a data processor, and/or a data transceiver. The upper portion of FIG. 15 shows a dorsal (e.g. top-down) view of the device and the lower portion of FIG. 15 shows a lateral (e.g. side) view of the device. In this example, proximal means closer to a person's shoulder and distal means farther from the person's shoulder.

In an example, the distal edge of a band (or strap) can be asymmetric with respect to the proximal edge of the band. In an example, the proximal edge of the band can be coplanar (e.g. a circle or oval in a single plane), but the distal edge of the band can be arcuate (spanning multiple planes). In an example, there can be a protrusion (e.g. bump or bulge) which protrudes distally out from the band on the dorsal side of the person's wrist (and/or forearm). In an example, a display screen (and/or watch housing) can be connected to this protrusion. In an example, this protrusion can have an arcuate (e.g. semicircular) shape. In an example, this protrusion can have a polygonal (e.g. quadrilateral) shape.

In an example, a band (or strap) can comprise a continuous piece of flexible polymer material. In an example, a band (or strap) can comprise a series of flexibly-connected rigid components (e.g. links or housings). In an example, a band (or strap) can be made from a breathable fabric and/or textile. In an example, a band (or strap) can comprise an alternating series of rigid components and flexible (e.g. elastic) components (e.g. alternating between rigid and flexible components) around (at least a portion of) the circumference of a person's wrist (and/or forearm). In an example, flexible components in this series can further comprise flexible electroconductive pathways.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array. Relevant variations discussed elsewhere in this disclosure or priority-linked disclosures can also be applied to this example.

FIG. 16 shows two views of a wearable device (e.g. a band, smart watch, or watch band) with neuromuscular activity (e.g. EMG) sensors comprising: an undulating band (or strap) 1603 which is configured to be worn around at least 50% of a circumference of a person's wrist (and/or forearm) 1601; a plurality of neuromuscular activity (e.g. EMG) sensors (including sensor 1604) on the band; and a display screen (and/or watch housing) 1602 which is held on the wrist (and/or forearm) by the band. In an example, the device can further comprise a battery, an amplifier, a data processor, and/or a data transceiver. The upper portion of FIG. 16 shows a dorsal (e.g. top-down) view of the device and the lower portion of FIG. 16 shows a lateral (e.g. side) view of the device. In this example, proximal means closer to a person's shoulder and distal means farther from the person's shoulder.

In an example, a band (or strap) can have a shape which undulates as it curves around (a portion of) the circumference of a person's wrist (and/or forearm). In an example, a band (or strap) can have sinusoidal undulations as it curves around, a portion of) the circumference of a person's wrist (and/or forearm). In an example, the proximal and distal edges of a band (or strap) can have (sinusoidal) undulations as they curve around (a portion of) the circumference of a person's wrist (and/or forearm). In an example, an undulating band (or strap) can have a constant width as it curves around a person's wrist (and/or forearm). In an example, the proximal and distal edges of the band can both be sinusoidal with the same sinusoidal phase with respect to radial locations. In another example, an undulating band (or strap) can have a variable width. In another example, the proximal and distal edges of the band can be sinusoidal with the opposite (e.g. symmetric) sinusoidal phases with respect to radial locations.

In an example, a band (or strap) can comprise a continuous piece of flexible polymer material. In an example, a band (or strap) can comprise a series of flexibly-connected rigid components (e.g. links or housings). In an example, a band (or strap) can be made from a breathable fabric and/or textile. In an example, a band (or strap) can comprise an alternating series of rigid components and flexible (e.g. elastic) components (e.g. alternating between rigid and flexible components) around (at least a portion of) the circumference of a person's wrist (and/or forearm). In an example, flexible components in this series can further comprise flexible electroconductive pathways.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array. Relevant variations discussed elsewhere in this disclosure or priority-linked disclosures can also be applied to this example.

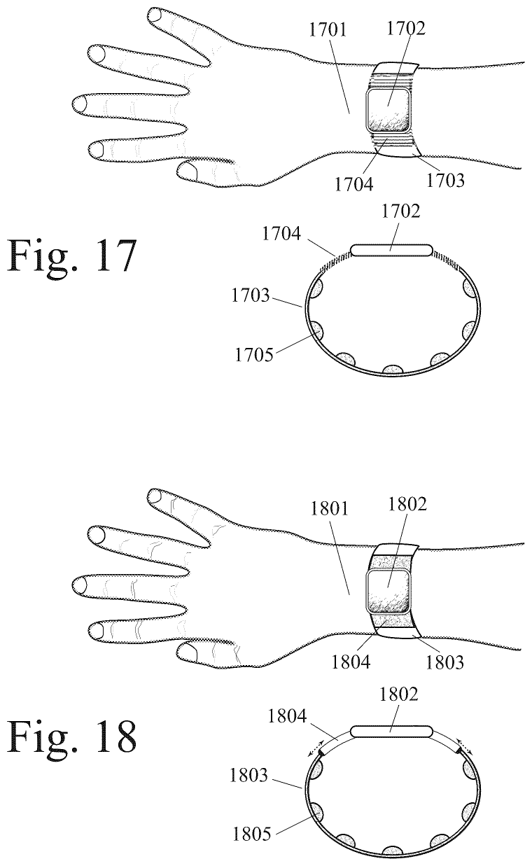

FIG. 17 shows two views of a wearable device (e.g. a band, smart watch, or watch band) with neuromuscular activity (e.g. EMG) sensors comprising: a band (or strap) 1703 which is configured to be worn around at least 50% of a circumference of a person's wrist (and/or forearm) 1701, wherein the band (or strap) further comprises a plurality of expandable sections (including 1704); a plurality of neuromuscular activity (e.g. EMG) sensors (including sensor 1705) on the band; and a display screen (and/or watch housing) 1702 which is held on the wrist (and/or forearm) by the band. In an example, the device can further comprise a battery, an amplifier, a data processor, and/or a data transceiver. The upper portion of FIG. 17 shows a dorsal (e.g. top-down) view of the device and the lower portion of FIG. 17 shows a lateral (e.g. side) view of the device. In this example, proximal means closer to a person's shoulder and distal means farther from the person's shoulder.

In an example, a band (or strap) can have two expandable sections can collectively span between 5% and 30% of the circumference of the person's wrist (and/or forearm). In an example, a band (or strap) can have two expandable sections which connect to a display screen (and/or watch housing). In an example, a band (or strap) can have two expandable sections which connect to sides of a display screen (and/or watch). In an example, a band (or strap) can comprise a non-expandable section on the portion of the band which spans the ventral surface of a person's wrist (and/or forearm) and two expandable sections which between the non-expandable section and a display screen (and/or watch housing) on the dorsal surface of the person's wrist (and/or forearm). In an example, a band (or strap) can comprise a non-expandable section which is not directly connected to a display screen (and/or watch housing) and a plurality of expandable sections which connect the non-expandable section to the display screen (and/or watch housing). In another example, a band (or strap) can have only one expandable section which connects to only one side of a display screen (and/or watch).

In an example, an expandable section of a band can be elastic and/or stretchable. In an example, an expandable section of a band can be made with elastic and/or stretchable material. In an example, an expandable section of a band can be made with elastic and/or stretchable fabric. In an example, an expandable section of a band can be piezoelectric. In an example, an expandable section of a band can be piezoelectric, wherein the section is expanded or contracted by the transmission of electrical energy through the section. In an example, the length of an expandable section can be changed by the transmission of electrical energy. In another example, the length of an expandable section of a band can be adjusted by an electromagnetic actuator. In another example, an expandable section can comprise telescoping components, wherein the length of the section can be adjusted by inserting or extending telescoping components relative to each other.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array. Relevant variations discussed elsewhere in this disclosure or priority-linked disclosures can also be applied to this example.

FIG. 18 shows a wearable device (e.g. a band, smart watch, or watch band) with neuromuscular activity (e.g. EMG) sensors which is like the one shown in FIG. 17, except that the expandable sections are telescoping.

FIG. 18 shows two views of a wearable device (e.g. a band, smart watch, or watch band) with neuromuscular activity (e.g. EMG) sensors comprising: a band (or strap) 1803 which is configured to be worn around at least 50% of a circumference of a person's wrist (and/or forearm) 1801, wherein the band (or strap) further comprises a plurality of telescoping expandable sections (including 1804); a plurality of neuromuscular activity (e.g. EMG) sensors (including sensor 1805) on the band; and a display screen (and/or watch housing) 1802 which is held on the wrist (and/or forearm) by the band. In an example, the device can further comprise a battery, an amplifier, a data processor, and/or a data transceiver. The upper portion of FIG. 18 shows a dorsal (e.g. top-down) view of the device and the lower portion of FIG. 18 shows a lateral (e.g. side) view of the device. In this example, proximal means closer to a person's shoulder and distal means farther from the person's shoulder.

In this example, an expandable section comprises telescoping components. In this example, the length an expandable section (and thus the overall length of the band) can be adjusted by moving telescoping components relative to each other. For example, the length of an expandable section can be increased by extending a first telescoping component out from second telescoping component and the length of the expandable section can be decreased by inserting the first telescoping component into the second telescoping component. In an example, an expandable section can further comprise a spring, piston, or elastic mechanism which causes tension (e.g. resists compression and/or extension) between the first and second telescoping components. In an example, a non-expandable section of a band can be inserted into an opening in a telescoping component of the band, thereby changing the overall length of the band. Relevant variations discussed elsewhere in this disclosure or priority-linked disclosures can also be applied to this example.

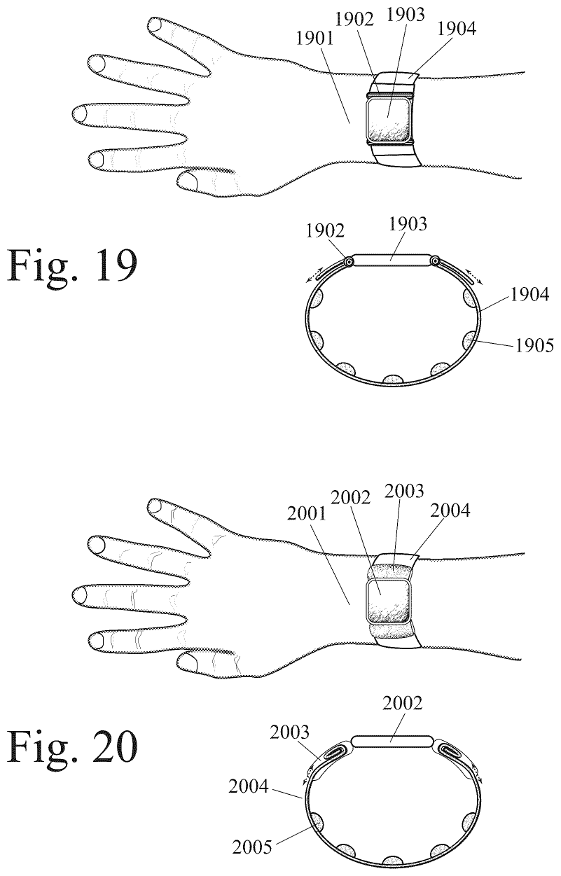

FIG. 19 shows two views of a wearable device (e.g. a band, smart watch, or watch band) with neuromuscular activity (e.g. EMG) sensors comprising: a band (or strap) 1904 which is configured to be worn around at least 50% of a circumference of a person's wrist (and/or forearm) 1901; at least one longitudinal member (e.g. roller or axle) 1902, wherein an end of the band curves (e.g. loops) around the longitudinal member to overlap a portion the band; a plurality of neuromuscular activity (e.g. EMG) sensors (including sensor 1905) on the band; and a display screen (and/or watch housing) 1903 which is held on the wrist (and/or forearm) by the band. In an example, the device can further comprise a battery, an amplifier, a data processor, and/or a data transceiver. The upper portion of FIG. 19 shows a dorsal (e.g. top-down) view of the device and the lower portion of FIG. 19 shows a lateral (e.g. side) view of the device. In this example, proximal means closer to a person's shoulder and distal means farther from the person's shoulder.

In an example, a longitudinal member can be cylindrical. In an example, a longitudinal member can be a cylindrical roller or axle. In an example, the longitudinal axis of a longitudinal member can be orthogonal to the circumference of the band. In an example, an end of the band can curve (loop) around a longitudinal member to double back on (and overlap a portion of) the rest of the band. In an example, the length of the end of the band which doubles back and overlaps the rest of the band can be adjusted. In an example, the length of the rest of the band which (partially) encircles a person's wrist (and/or forearm) can be adjusted by adjusting the length (amount) of the band which doubles back and overlaps the rest of the band. In an example, there can be two longitudinal members (e.g. rollers or axles) on the device, one on each side of a display screen (and/or watch housing). In an example, a band can be connected to the sides of a display screen (and/or watch housing) by curving (looping) around two such longitudinal members.

In an example, the fit of the band around a person's wrist (and/or forearm) can be adjusted by changing the length of the band which doubles back around a longitudinal member and overlaps the rest of the band. In an example, pulling more of the end of a band around the longitudinal member reduces the length of the rest of the band which encircles a person's wrist (and/or forearm). In an example, pulling less of the end of a band around the longitudinal member increases the length of the rest of the band which encircles a person's wrist (and/or forearm). In an example, this enables adjustment of the band (e.g. making it tighter or looser) without substantively changing the configuration of neuromuscular activity (EMG) sensors on a ventral surface of the person's wrist (and/or forearm).

In an example, the device can also comprise a locking mechanism (e.g. a loop, latch, pin, buckle, hook-and-loop, or magnetic mechanism) which keeps (e.g. locks) an end portion of a band which has curved (looped) around a longitudinal member in place where it overlaps the rest of the band. In an example, the device can also comprise a locking mechanism (e.g. a loop, latch, pin, buckle, or magnetic mechanism) which holds an end portion of a band which has curved (looped) around a longitudinal member against the rest of the band.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array. Relevant variations discussed elsewhere in this disclosure or priority-linked disclosures can also be applied to this example.

FIG. 20 shows a wearable device (e.g. a band, smart watch, or watch band) with neuromuscular activity (e.g. EMG) sensors which is like the one shown in FIG. 19, except that at least one end of a band is adjustably-coiled within a coil housing in order to change the fit of the band on a person's wrist (and/or forearm).

FIG. 20 shows two views of a wearable device (e.g. a band, smart watch, or watch band) with neuromuscular activity (e.g. EMG) sensors comprising: a band (or strap) 2004 which is configured to be worn around at least 50% of a circumference of a person's wrist (and/or forearm) 2001; at least one coil housing 2003, wherein an end of the band is adjustably-coiled within the coil housing to change the length of the rest of the band which encircles (a portion of) the circumference of the person's wrist (and/or forearm); a plurality of neuromuscular activity (e.g. EMG) sensors (including sensor 2005) on the band; and a display screen (and/or watch housing) 2002 which is held on the wrist (and/or forearm) by the band. In an example, the device can further comprise a battery, an amplifier, a data processor, and/or a data transceiver. The upper portion of FIG. 20 shows a dorsal (e.g. top-down) view of the device and the lower portion of FIG. 20 shows a lateral (e.g. side) view of the device. In this example, proximal means closer to a person's shoulder and distal means farther from the person's shoulder.

In an example, the length of a band which (partially) encircles a person's wrist (and/or forearm) can be decreased by coiling more of the end of the band within a coil housing. In an example, the length of a band which (partially) encircles a person's wrist (and/or forearm) can be decreased by coiling more of the end of the band around a roller or axle within a coil housing. In an example, the length of a band which (partially) encircles a person's wrist (and/or forearm) can be increased by coiling less of the end of the band within a coil housing. In an example, the length of a band which (partially) encircles a person's wrist (and/or forearm) can be increased by coiling less of the end of the band around a roller or axle within a coil housing.

In an example, a device can have one coil housing within which one end of a band is coiled. In an example, a device can have two coil housings within which two ends, respectively (e.g. one end in each coil housing), of a band are coiled. In an example, a coil housing can be located on one side of a display screen (and/or watch housing). In an example, a coil housing can be between a band and a display screen (and/or watch housing). In an example, one or more housings can be the means by which a band is connected to a display screen (and/or watch housing).

In an example, an end of band can be coiled within a coil housing by rotating an axle or roller around which the end is coiled. In an example, an end of band can be adjustably-coiled within a coil housing by manually rotating an axle or roller around which the end is coiled. In an example, an end of band can be adjustably-coiled within a coil housing by an actuator which automatically rotates an axle or roller around which the end is coiled. In an example, an end of band can be adjustably-coiled within a coil housing by an actuator which automatically rotates an axle or roller around which the end is coiled in response to data from the neuromuscular activity (EMG) sensors or other sensors on the band. In an example, an axle or roller around which an end of a band is coiled in a coil housing can be connected to a spring which keeps the end of the band in tension. Relevant variations discussed elsewhere in this disclosure or priority-linked disclosures can also be applied to this example.

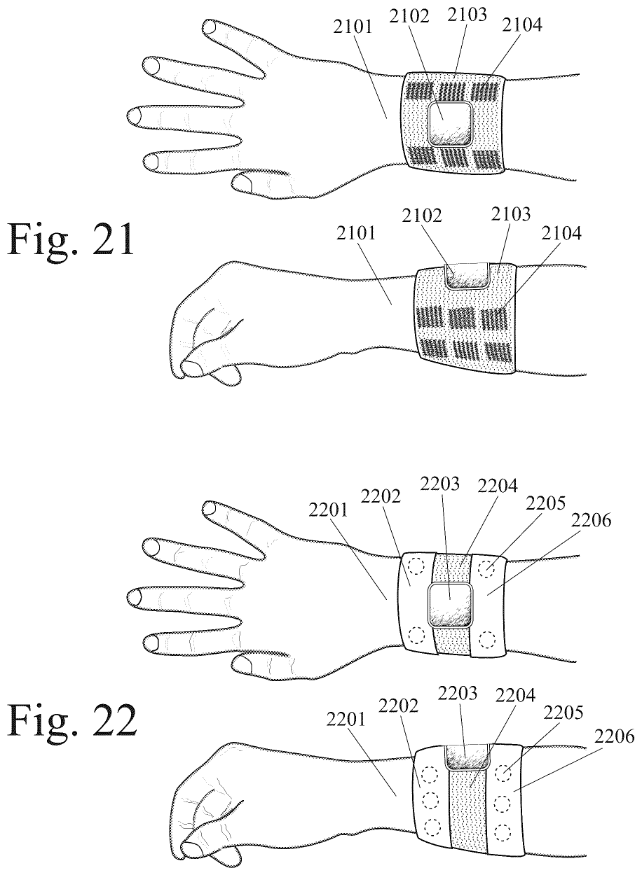

FIG. 21 shows two views of a wearable device with neuromuscular activity (e.g. EMG) sensors comprising: a fabric sleeve (or cuff) 2103 which is configured to be worn around at least 50% of a circumference of a person's wrist (and/or forearm) 2101, wherein the fabric sleeve (or cuff) further comprises a plurality of neuromuscular activity (e.g. EMG) sensors (including sensor 2104); and a display screen (and/or watch housing) 2102 which is held on the wrist (and/or forearm) by the fabric sleeve. In an example, the device can further comprise a battery, an amplifier, a data processor, and/or a data transceiver. The upper portion of FIG. 21 shows a dorsal (e.g. top-down) view of the device and the lower portion of FIG. 21 shows a lateral (e.g. side) view of the device. In this example, proximal means closer to a person's shoulder and distal means farther from the person's shoulder.

In an example, neuromuscular activity (EMG) sensors can be formed by weaving, sewing, braiding, or embroidering electroconductive yarns, threads, or fibers into the fabric used to create the sleeve (or cuff). In an example, neuromuscular activity (EMG) sensors can be formed by selectively weaving, sewing, braiding, or embroidering electroconductive yarns, threads, or fibers into selected areas of the fabric used to create the sleeve before the sleeve is formed. In an example, neuromuscular activity (EMG) sensors can be formed by sewing, braiding, or embroidering electroconductive yarns, threads, or fibers onto the sleeve after the sleeve is formed. In an example, neuromuscular activity (EMG) sensors can be formed by printing electroconductive ink onto the sleeve. In an example, neuromuscular activity (EMG) sensors can be formed by adhering electroconductive strips or patches onto the sleeve. In an example, the sleeve (or cuff) can be made with elastic material. In an example, the sleeve (or cuff) can be made with elastic yarns, threads, or fibers.

In an example, neuromuscular activity (e.g. EMG) sensors can be distributed around the entire (circumferential or partially-circumferential) inner perimeter of the sleeve. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the sleeve, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, a display screen (and/or watch housing) can be an integral part of a device. In an example, a device including the sleeve, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can function as a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a sleeve which functions as a watch band (or strap) can span between 50% and 90% of the circumference of a person's wrist (and/or forearm). Relevant variations discussed elsewhere in this disclosure or priority-linked disclosures can also be applied to this example.

FIG. 22 shows two views of a wearable device (e.g. a band, smart watch, or watch band) with neuromuscular activity (e.g. EMG) sensors comprising: a three-ring band which is configured to be worn around at least 50% of a circumference of a person's wrist (and/or forearm) 2201, wherein the three-ring band further comprises a proximal polymer or metal ring 2206, a distal polymer or metal ring 2202, and a middle fabric ring 2204 between the proximal and distal rings; a plurality of neuromuscular activity (e.g. EMG) sensors (including sensor 2205) on the band; and a display screen (and/or watch housing) 2203 which is held on the wrist (and/or forearm) by the band. In an example, the device can further comprise a battery, an amplifier, a data processor, and/or a data transceiver. The upper portion of FIG. 22 shows a dorsal (e.g. top-down) view of the device and the lower portion of FIG. 22 shows a lateral (e.g. side) view of the device. In this example, proximal means closer to a person's shoulder and distal means farther from the person's shoulder.

In an example, the proximal ring and/or the distal ring can be made from a polymer. In an example, the proximal ring and/or the distal ring can be made from interconnected metal links, chains, or housings. In an example, the middle ring can be made from an air-permeable fabric and/or textile. In an example, the middle ring can be made from a stretchable and/or elastic fabric and/or textile. In an example, the middle ring can span between 75% and 90% of the circumference of a person's wrist (and/or forearm). In an example, the three rings can all have the same width. In an example, the middle ring can be wider than either the proximal ring or the distal ring.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, neuromuscular activity (e.g. EMG) sensors on a band can be arrayed in one or more rings. In an example, neuromuscular activity (e.g. EMG) sensors on a band can be aligned along one or more circumferential rings around the band and/or the person's wrist (and/or forearm). In an example, neuromuscular activity (e.g. EMG) sensors on a band can be arrayed along a single ring. In an example, neuromuscular activity (e.g. EMG) sensors on a band can be aligned along a single (e.g. proximally-to-distal central) circumferential ring around the band and/or the person's wrist (and/or forearm). In an example, neuromuscular activity (e.g. EMG) sensors on a band can be arrayed along two rings. In an example, neuromuscular activity (e.g. EMG) sensors on a band can be aligned along two circumferential rings around the band and/or the person's wrist (and/or forearm).

In an example, an array of neuromuscular activity (e.g. EMG) sensors on a band can comprise a ring-and-row array, wherein a ring is around the circumference of a person's wrist (and/or forearm) and a row is orthogonal to a ring. In an example, there can be three or more neuromuscular activity (e.g. EMG) sensors in each ring of such an array. In an example, there can be six or more neuromuscular activity (e.g. EMG) sensors in each ring of such an array. In an example, there can be two or more neuromuscular activity (e.g. EMG) sensors in each row of such an array. In an example, there can be three or more electrodes in each ring of such an array.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

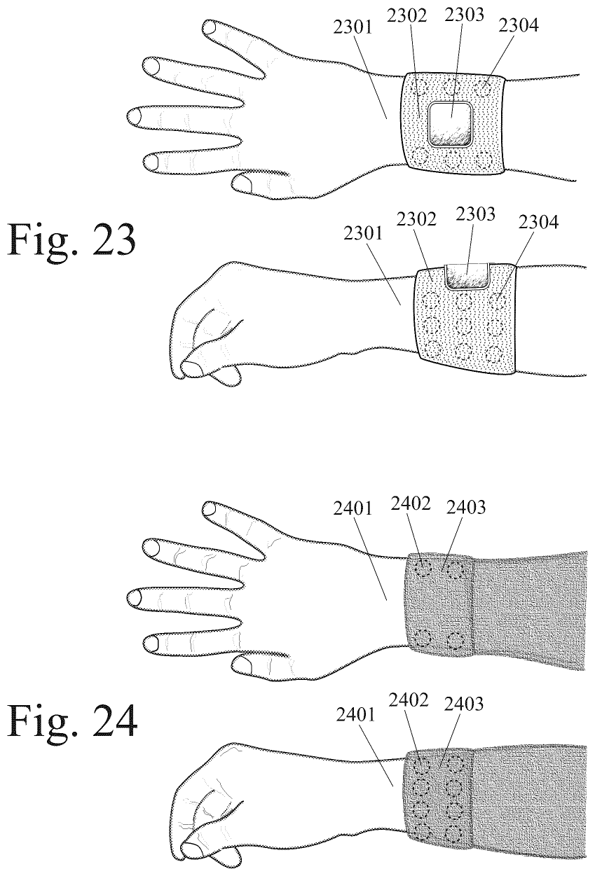

FIG. 23 shows two views of a wearable device with neuromuscular activity (e.g. EMG) sensors comprising: a fabric sleeve (or cuff) 2302 which is configured to be worn around at least 50% of a circumference of a person's wrist (and/or forearm) 2301; a plurality of neuromuscular activity (e.g. EMG) sensors (including sensor 2304) attached to the sleeve; and a display screen (and/or watch housing) 2303 which is held on the wrist (and/or forearm) by the fabric sleeve. In an example, the device can further comprise a battery, an amplifier, a data processor, and/or a data transceiver. The upper portion of FIG. 23 shows a dorsal (e.g. top-down) view of the device and the lower portion of FIG. 23 shows a lateral (e.g. side) view of the device. In this example, proximal means closer to a person's shoulder and distal means farther from the person's shoulder.

In an example, neuromuscular activity (EMG) sensors can be attached to a sleeve (or cuff) by adhesion (e.g. gluing). In an example, neuromuscular activity (EMG) sensors can be attached to a sleeve by sewing. In an example, neuromuscular activity (EMG) sensors can be attached to the sleeve by printing. In an example, neuromuscular activity (EMG) sensors can be attached to a sleeve by snaps. In an example, neuromuscular activity (EMG) sensors can be attached to a sleeve by pins.

In an example, neuromuscular activity (e.g. EMG) sensors can be distributed around the entire (circumferential or partially-circumferential) inner perimeter of the sleeve. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the sleeve, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, a display screen (and/or watch housing) can be an integral part of a device. In an example, a device including the sleeve, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can function as a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a sleeve which functions as a watch band (or strap) can span between 50% and 90% of the circumference of a person's wrist (and/or forearm). Relevant variations discussed elsewhere in this disclosure or priority-linked disclosures can also be applied to this example.

FIG. 24 shows two views of a wearable device with neuromuscular activity (e.g. EMG) sensors comprising: a shirt sleeve (or cuff) 2403 which is configured to be worn on person's arm 2401; and a plurality of neuromuscular activity (e.g. EMG) sensors (including sensor 2402) on the sleeve (or cuff). In an example, the device can further comprise a battery, an amplifier, a data processor, and/or a data transceiver. The upper portion of FIG. 24 shows a dorsal (e.g. top-down) view of the device and the lower portion of FIG. 24 shows a lateral (e.g. side) view of the device.

In an example, neuromuscular activity (EMG) sensors can be attached to a shirt sleeve (or cuff) by adhesion (e.g. gluing). In an example, neuromuscular activity (EMG) sensors can be attached to a sleeve by sewing or embroidering. In an example, neuromuscular activity (EMG) sensors can be attached to the sleeve by printing. In an example, neuromuscular activity (EMG) sensors can be attached to a sleeve by snaps. In an example, neuromuscular activity (EMG) sensors can be attached to a sleeve by pins. In an example, neuromuscular activity (EMG) sensors can be woven into the fabric used to a shirt, before the shirt is formed. In an example, neuromuscular activity (EMG) sensors can be made by weaving electroconductive threads, yarns, or fibers into fabric used to a shirt, before the shirt is formed.

In an example, neuromuscular activity (e.g. EMG) sensors can be distributed around the entire (circumferential or partially-circumferential) inner perimeter of the shirt sleeve (or cuff). In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the sleeve, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm). Relevant variations discussed elsewhere in this disclosure or priority-linked disclosures can also be applied to this example.

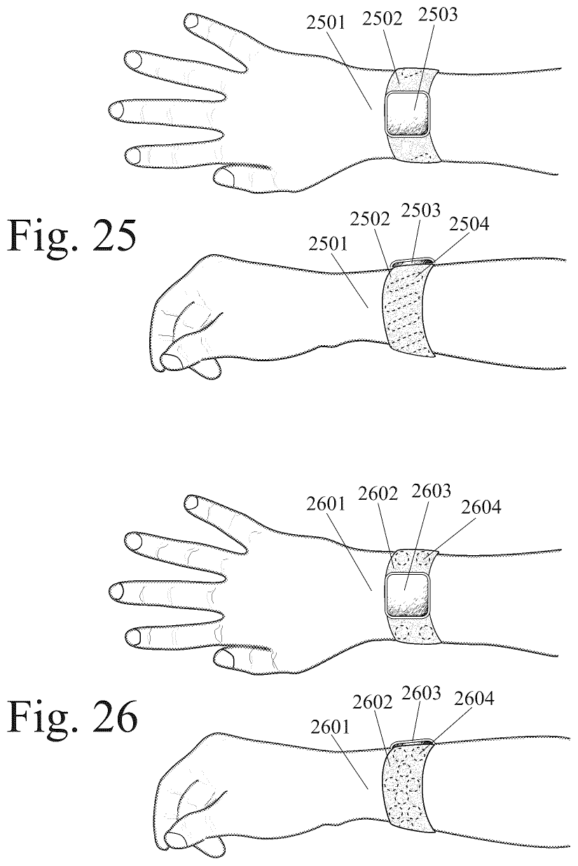

FIG. 25 shows two views of a wearable device (e.g. a band, smart watch, or watch band) with neuromuscular activity (e.g. EMG) sensors comprising: a band (or strap) 2502 which is configured to be worn around at least 50% of a circumference of a person's wrist (and/or forearm) 2501; a plurality of longitudinal neuromuscular activity (e.g. EMG) sensors (including sensor 2504) on the band, wherein longitudinal axes of the sensors intersect a circumference of the band at acute angles; and a display screen (and/or watch housing) 2503 which is held on the wrist (and/or forearm) by the band. In an example, the device can further comprise a battery, an amplifier, a data processor, and/or a data transceiver. The upper portion of FIG. 25 shows a dorsal (e.g. top-down) view of the device and the lower portion of FIG. 25 shows a lateral (e.g. side) view of the device.

In an example, longitudinal axes of longitudinal neuromuscular activity (EMG) sensors can intersect a circumference of the band at one or more angles between 30 and 60 degrees. In an example, longitudinal neuromuscular (EMG) sensors can be span the width of a band diagonally (e.g. not straight across between a proximal edge and a distal edge). In an example, longitudinal neuromuscular activity (EMG) sensors can have oblong, oval, or elliptical cross-sectional shapes. In an example, longitudinal neuromuscular activity (EMG) sensors can have quadrilateral cross-sectional shapes. In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

FIG. 26 shows two views of a wearable device (e.g. a band, smart watch, or watch band) with neuromuscular activity (e.g. EMG) sensors comprising: a band (or strap) 2602 which is configured to be worn around at least 50% of a circumference of a person's wrist (and/or forearm) 2601; a plurality of neuromuscular activity (EMG) sensors (including sensor 2604), wherein the plurality includes a first series of sensors distributed around a first circumferential line of the band and a second series of sensors distributed around a second circumferential line of the band, and wherein sensors in the first series and sensors in the second series are not laterally aligned (e.g. are not aligned along lines which are orthogonal with the circumference of the band); and a display screen (and/or watch housing) 2603 which is held on the wrist (and/or forearm) by the band. In an example, the device can further comprise a battery, an amplifier, a data processor, and/or a data transceiver. The upper portion of FIG. 26 shows a dorsal (e.g. top-down) view of the device and the lower portion of FIG. 26 shows a lateral (e.g. side) view of the device.

In an example, a plurality of neuromuscular activity (EMG) sensors can include two rings of sensors which are not laterally-aligned. In an example, a plurality of neuromuscular activity (EMG) sensors can include two staggered rings of sensors. In an example, a plurality of neuromuscular activity (EMG) sensors can include three or more rings of sensors which are not all laterally-aligned. In an example, a plurality of neuromuscular activity (EMG) sensors can include three staggered rings of sensors. In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

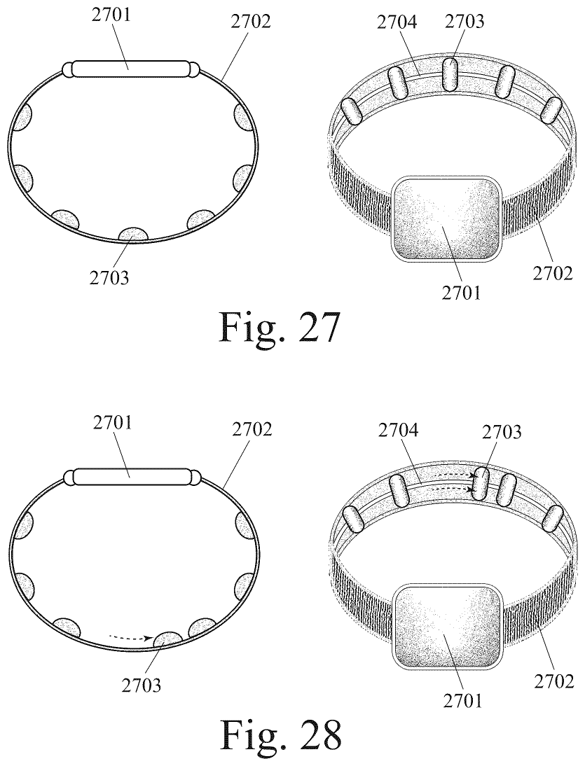

FIGS. 27 and 28 show lateral and oblique views, at two different times, of a wearable device (e.g. a band, smart watch, or watch band) with circumferentially-movable neuromuscular activity (e.g. EMG) sensors. Left portions of these figures show lateral views. Right portions of these figures show oblique views. FIG. 27 shows the device before a neuromuscular activity (EMG) sensor has been moved. FIG. 28 shows the device after a neuromuscular activity (EMG) sensor has been moved.

FIGS. 27 and 28 show a wearable device (e.g. a band, smart watch, or watch band) with circumferentially-movable neuromuscular activity (e.g. EMG) sensors comprising: a band (or strap) 2702 which is configured to be worn around at least 50% of a circumference of a person's wrist (and/or forearm); a channel, track, or ring 2704 around a circumference of the band (or strap); a plurality of circumferentially-movable neuromuscular activity (e.g. EMG) sensors (including sensor 2703) on the band, wherein one or more of the neuromuscular activity sensors can be selectively moved along the channel, track, or ring; and a display screen (and/or watch housing) 2701 which is held on the wrist (and/or forearm) by the band. In an example, the device can further comprise a battery, an amplifier, a data processor, and/or a data transceiver.

In an example, one or more neuromuscular activity (EMG) sensors can be manually moved along the channel, track, or ring. In an example, the device can further comprise an electromagnetic actuator which automatically moves one or more neuromuscular activity (EMG) sensors along the channel, track, or ring. In an example, distances between sensors on the band can be adjusted by selectively moving them along the channel, track, or ring in order to more accurately record the activity of specific muscles. In an example, distances between sensors on the band can be adjusted by selectively moving them along the channel, track, or ring in order to customize the sensor configuration to the anatomy of a specific person.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, neuromuscular activity (e.g. EMG) sensors can be distributed around the entire (circumferential or partially-circumferential) inner perimeter of the band. In an example, neuromuscular activity (e.g. EMG) sensors can be distributed around the entire circumference of a person's wrist (and/or forearm). In an example, neuromuscular activity (e.g. EMG) sensors can collectively span 100% of the circumference of the person's wrist (and/or forearm). In an example, neuromuscular activity (e.g. EMG) sensors can be on ventral and lateral portions of the perimeter of the band, wherein the ventral portion is the portion which spans the ventral surface of the person's wrist (and/or forearm) and the lateral portions span the sides of the person's wrist (and/or forearm) between the ventral and dorsal surfaces of the person's wrist (and/or forearm). In an example, neuromuscular activity (e.g. EMG) sensors can collectively span between 30% and 80% of the circumference of the person's wrist (and/or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

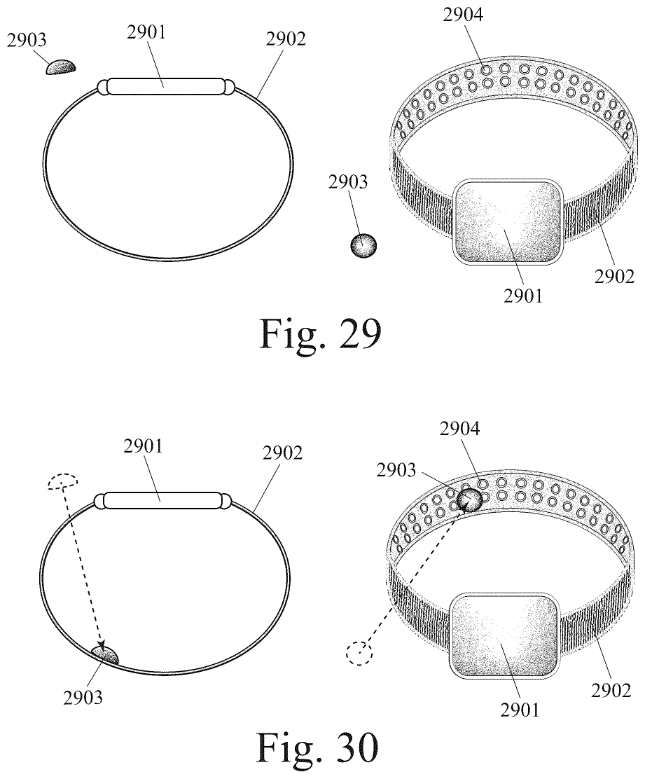

FIGS. 29 and 30 show lateral and oblique views, at two different times, of a wearable device (e.g. a band, smart watch, or watch band) with one or more modular neuromuscular activity (e.g. EMG) sensors. Left portions of these figures show lateral views. Right portions of these figures show oblique views. FIG. 29 shows the device before a modular neuromuscular activity (EMG) sensor has been attached to the device. FIG. 30 shows the device after a modular neuromuscular activity (EMG) sensor has attached to the device.

FIGS. 29 and 30 show a wearable device (e.g. a band, smart watch, or watch band) with modular neuromuscular activity (e.g. EMG) sensors comprising: a band (or strap) 2902 which is configured to be worn around at least 50% of a circumference of a person's wrist (and/or forearm); one or more modular neuromuscular activity (e.g. EMG) sensors 2903 which can be removably-attached to different attachment locations (including location 2904) on the band; and a display screen (and/or watch housing) 2901 which is held on the wrist (and/or forearm) by the band. In an example, the device can further comprise a battery, an amplifier, a data processor, and/or a data transceiver.

In an example, a modular neuromuscular activity (e.g. EMG) sensor can be removably-attached to a band at different locations at different times by an attachment mechanism selected from the group consisting of: clasp, clip, hook, magnet, pin, plug, port, and snap. In an example, a band can have an array of ports into which one or more sensors can be inserted. In an example, a band can have a ring-and-row array of attachment locations (e.g. ports) to which one or more sensors can be removably attached. In an example, this modularity can enable configuring a plurality of sensors on the band to more accurately record the activity of specific muscles. In an example, this modularity can enable customizing a configuration of sensors to the anatomy of a specific person.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

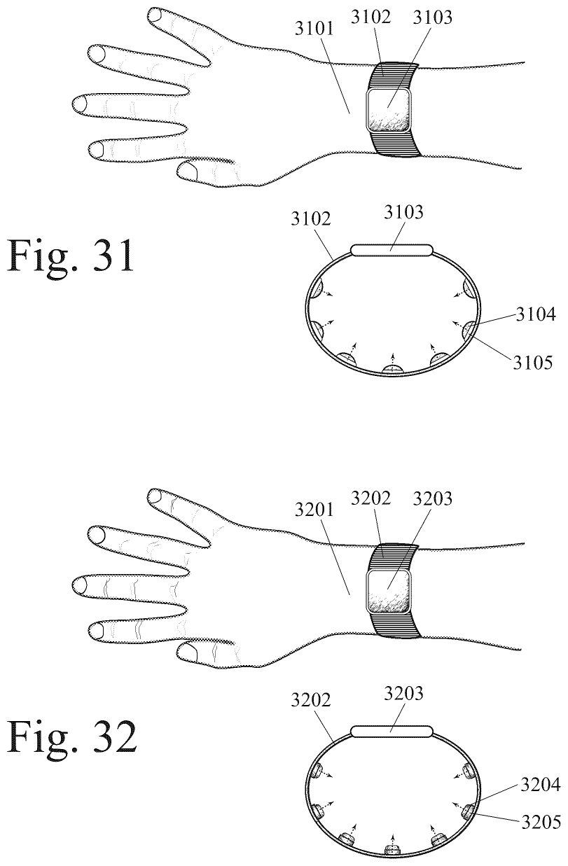

FIG. 31 shows two views a wearable device (e.g. a band, smart watch, or watch band) with neuromuscular activity (e.g. EMG) sensors whose protrusion distances toward a person's wrist (and/or forearm) can be selectively-adjusted by expansion of one or more expandable (e.g. inflatable) chambers. The upper portion of FIG. 31 shows a dorsal (e.g. top down) view of this device. The lower portion of FIG. 31 shows a lateral (e.g. side) view of this device.

FIG. 31 shows a wearable device (e.g. a band, smart watch, or watch band) with neuromuscular activity (e.g. EMG) sensors comprising: a band (or strap) 3102 which is configured to be worn around at least 50% of a circumference of a person's wrist (and/or forearm) 3101; a plurality of neuromuscular activity (EMG) sensors (including 3104) on the band; a plurality of expandable (e.g. inflatable) chambers (including 3105) between the sensors and the band, wherein selective expansion (e.g. inflation) of one or more of the expandable chambers pushes one or more of the sensors away from the band toward the person's wrist (and/or forearm); and a display screen (and/or watch housing) 3103 which is held on the wrist (and/or forearm) by the band. In an example, the device can further comprise a battery, an amplifier, a data processor, and/or a data transceiver.

In an example, a selected set of one or more neuromuscular activity (EMG) sensors can have a first configuration in which they protrude a first distance from the band and second configuration in which they protrude a second distance from the band, wherein the second distance is greater than the first distance. In an example, the selected set of one or more neuromuscular activity (EMG) sensors can be changed from its first configuration to its second configuration by expansion (e.g. inflation) of one or more expandable chambers between the sensors and the band. In an example, there can be one expandable chamber for each sensor. In an example, an expandable chamber can be expanded by being filled with a flowable substance (e.g. a gas, liquid, or gel). In an example, one or more sensors can be changed back from their second configurations to their first configurations by shrinking (e.g. deflating) expandable chambers associated with them.

In an example, the distance between a neuromuscular activity (EMG) sensor on a band and a person's wrist (and/or forearm) can be adjusted by expansion or shrinkage of an expandable chamber between the sensor and the band. In an example, the distance between a neuromuscular activity (EMG) sensor on a band and a person's wrist (and/or forearm) can be adjusted by inflation or deflation of an expandable chamber between the sensor and the band. In an example, the distance between a neuromuscular activity (EMG) sensor on a band and a person's wrist (and/or forearm) can be adjusted by pumping a flowable substance into, or out of, an expandable chamber between the sensor and the band.

In an example, pressure between a neuromuscular activity (EMG) sensor and a person's wrist (and/or forearm) can be adjusted by expansion or shrinkage of an expandable chamber between the sensor and the band. In an example, pressure between a neuromuscular activity (EMG) sensor and a person's wrist (and/or forearm) can be adjusted by inflation or deflation of an expandable chamber between the sensor and the band. In an example, pressure between a neuromuscular activity (EMG) sensor and a person's wrist (and/or forearm) can be adjusted by pumping a flowable substance into, or out of, an expandable chamber between the sensor and the band.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

FIG. 32 shows two views a wearable device (e.g. a band, smart watch, or watch band) with neuromuscular activity (e.g. EMG) sensors whose protrusion distances toward a person's wrist (and/or forearm) can be selectively-adjusted by activation of hydraulic pistons or electromagnetic solenoids. The upper portion of FIG. 32 shows a dorsal (e.g. top down) view of this device. The lower portion of FIG. 32 shows a lateral (e.g. side) view of this device.

FIG. 32 shows a wearable device (e.g. a band, smart watch, or watch band) with neuromuscular activity (e.g. EMG) sensors comprising: a band (or strap) 3202 which is configured to be worn around at least 50% of a circumference of a person's wrist (and/or forearm) 3201; a plurality of neuromuscular activity (EMG) sensors (including 3204) on the band; a plurality of hydraulic pistons or electromagnetic solenoids (including 3205) between the sensors and the band, wherein selective activation of one or more of the pistons or solenoids pushes one or more of the sensors away from the band toward the person's wrist (and/or forearm); and a display screen (and/or watch housing) 3203 which is held on the wrist (and/or forearm) by the band. In an example, the device can further comprise a battery, an amplifier, a data processor, and/or a data transceiver.

In an example, a selected set of one or more neuromuscular activity (EMG) sensors can have a first configuration in which they protrude a first distance from the band and second configuration in which they protrude a second distance from the band, wherein the second distance is greater than the first distance. In an example, the selected set of one or more neuromuscular activity (EMG) sensors can be changed from its first configuration to its second configuration by activation of one or more hydraulic pistons or electromagnetic solenoids between the sensors and the band. In an example, there can be one piston or solenoid for each sensor.

In an example, the distance between a neuromuscular activity (EMG) sensor on a band and a person's wrist (and/or forearm) can be adjusted by activation of a piston or solenoid between the sensor and the band. In an example, the pressure between a neuromuscular activity (EMG) sensor on a band and a person's wrist (and/or forearm) can be adjusted by activation of a piston or solenoid between the sensor and the band.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

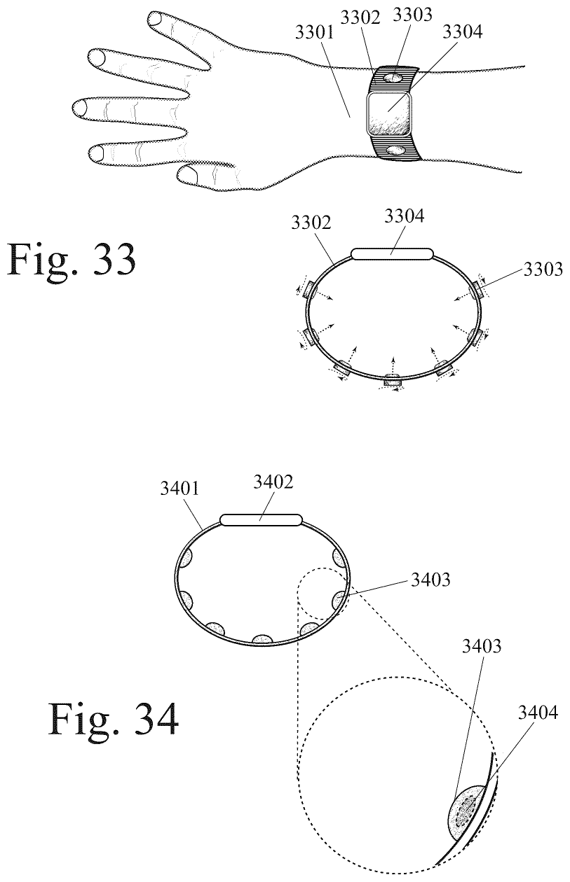

FIG. 33 shows two views a wearable device (e.g. a band, smart watch, or watch band) with threaded neuromuscular activity (e.g. EMG) sensors whose protrusion distances (out from a band) toward a person's wrist (and/or forearm) can be selectively-adjusted by rotation. The upper portion of FIG. 33 shows a dorsal (e.g. top down) view of this device. The lower portion of FIG. 33 shows a lateral (e.g. side) view of this device.

FIG. 33 shows a wearable device (e.g. a band, smart watch, or watch band) with neuromuscular activity (e.g. EMG) sensors comprising: a band (or strap) 3302 which is configured to be worn around at least 50% of a circumference of a person's wrist (and/or forearm) 3301; a plurality of helically-threaded neuromuscular activity (EMG) sensors (including 3303) on the band, wherein rotation of a helically-threaded neuromuscular activity (EMG) sensor changes the distance by which the sensor protrudes out from the band toward the person's wrist (and/or forearm); and a display screen (and/or watch housing) 3304 which is held on the wrist (and/or forearm) by the band. In an example, the device can further comprise a battery, an amplifier, a data processor, and/or a data transceiver.

In an example, a selected set of one or more threaded neuromuscular activity (EMG) sensors can have a first configuration in which they protrude a first distance out from the band and second configuration in which they protrude a second distance out from the band, wherein the second distance is greater than the first distance. In an example, the selected set of one or more neuromuscular activity (EMG) sensors can be changed from their first configuration to their second configuration by being rotated. In an example, helical-threads on a sensor can engage corresponding helical threads on a hole (e.g. opening) in the band through which the sensor is inserted, when the sensor is rotated within the hole. In an example, this rotation can be done manually. In an example, this rotation can be done automatically by an electromagnetic actuator.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

FIG. 34 shows lateral and selected close-up views a wearable device (e.g. a band, smart watch, or watch band) with neuromuscular activity (e.g. EMG) sensors, wherein there are (expandable) fluid-filled chambers within the sensors. The upper-left portion of FIG. 34 shows a lateral view of this device. The lower-right portion of FIG. 34 shows a close-up cross-sectional view of one sensor on the device.

FIG. 34 shows a wearable device (e.g. a band, smart watch, or watch band) with neuromuscular activity (e.g. EMG) sensors comprising: a band (or strap) 3401 which is configured to be worn around at least 50% of a circumference of a person's wrist (and/or forearm); a plurality of neuromuscular activity (EMG) sensors (including 3403) on the band, wherein there are (expandable) fluid-filled chambers (including 3404) within the sensors; and a display screen (and/or watch housing) 3402 which is held on the wrist (and/or forearm) by the band. In an example, the device can further comprise a battery, an amplifier, a data processor, and/or a data transceiver.

In an example, a selected set of one or more neuromuscular activity (EMG) sensors can have a first configuration in which they protrude a first distance out from the band and second configuration in which they protrude a second distance out from the band, wherein the second distance is greater than the first distance. In an example, the selected set of one or more neuromuscular activity (EMG) sensors can be changed from their first configuration to their second configuration by expansion of fluid-filled chambers inside them. In an example, a fluid-filled chamber can be expanded by pumping more fluid into the chamber. In an example, fluid can be manually pumped into a fluid-filled chamber. In an example, fluid can be automatically pumped into a fluid-filled chamber (e.g. by an electromagnetic actuator).

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

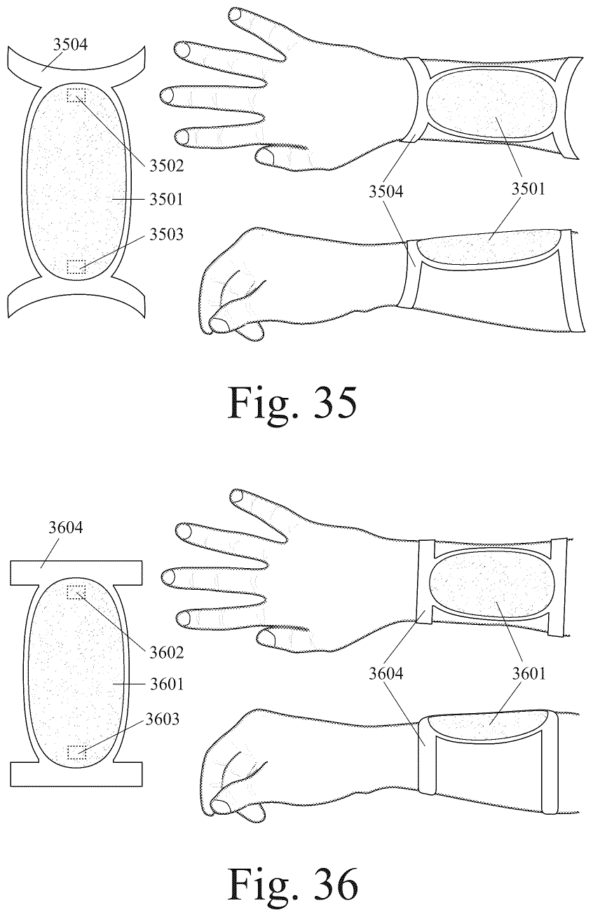

FIG. 35 shows an example of a wearable computing device for the wrist and/or arm comprising: longitudinal arcuate display member 3501; data control unit 3502; sensor 3503; and longitudinal flared-two-strap attachment member 3504. The left portion of FIG. 35 shows a detailed top-down view of the device by itself. The upper-right portion of FIG. 35 shows a top-down view of this device on a person's forearm. The lower-right portion of FIG. 35 shows a side view of this device on a person's forearm. In an example, sensor 3503 and/or other sensors which are part of the device can be electromyography (EMG) sensors. In an example, the configuration and/or movement of a person's fingers, hand, and/or arm causes electromagnetic signals from the person's muscles which are detected by the EMG sensors.

In an example of a wearable computing device for the wrist and/or arm can comprise: a longitudinal arcuate display member; a data control unit; electromyography (EMG) sensors; and a longitudinal flared-two-strap attachment member. In an example, electromyography (EMG) sensors can be on the longitudinal flared-two-strap attachment member. In an example, electromyography (EMG) sensors can be around the circumference of the longitudinal flared-two-strap attachment member. In an example, electromyography (EMG) sensors can be on the ventral portion of the longitudinal flared-two-strap attachment member.

In an example, a longitudinal flared-two-strap attachment member can be configured to hold a longitudinal arcuate display member, a data control unit, and one or more sensors within three inches of the surface of a person's body. In an example, a longitudinal flared-two-strap attachment member can further comprise two straps or bands which each span the circumference of the person's wrist and/or arm. This holds the device onto a person's arm at two different locations along the longitudinal axis of the person's forearm.

In an example, these straps or bands can be flexible. In an example, these straps or bands can each have portions which interconnect so as to the fasten longitudinal flared-two-strap attachment member around the person's wrist and/or arm in two different longitudinal locations. In an example, these straps or bands can further comprise clips, clasps, snaps, buckles, or hook-and-eye connectors. In an example, these straps or bands can be stretched or expanded to a sufficiently-large circumference so that they can be slipped over a person's hand onto the person's wrist and/or arm. Relevant variations discussed elsewhere in this disclosure or priority-linked disclosures can also be applied to this example.

FIG. 36 shows an example of a wearable computing device for the wrist and/or arm comprising: longitudinal arcuate display member 3601; data control unit 3602; sensor 3603; and longitudinal two-strap attachment member 3604. The left portion of FIG. 36 shows a detailed top-down view of the device by itself. The upper-right portion of FIG. 36 shows a top-down view of this device worn on a person's forearm. The lower-right portion of FIG. 36 shows a side view of this device worn on a person's forearm. This example is like the example shown in FIG. 35 except that the two straps or bands of the longitudinal two-strap attachment member do not flare outwards. Accordingly, the distance between the two straps or bands is substantially the same on both opposing sides of the arm. In an example, sensor 3603 and/or other sensors which are part of the device can be electromyography (EMG) sensors. In an example, the configuration and/or movement of a person's fingers, hand, and/or arm causes electromagnetic signals from the person's muscles which are detected by the EMG sensors.

In an example, a wearable computing device for the wrist and/or arm can comprise: a longitudinal arcuate display member; a data control unit; electromyography (EMG) sensors; and a longitudinal two-strap attachment member. In an example, electromyography (EMG) sensors can be on the longitudinal two-strap attachment member. In an example, electromyography (EMG) sensors can be around the circumference of the longitudinal two-strap attachment member. In an example, electromyography (EMG) sensors can be on the ventral portion of the longitudinal two-strap attachment member. Relevant variations discussed elsewhere in this disclosure or priority-linked disclosures can also be applied to this example.

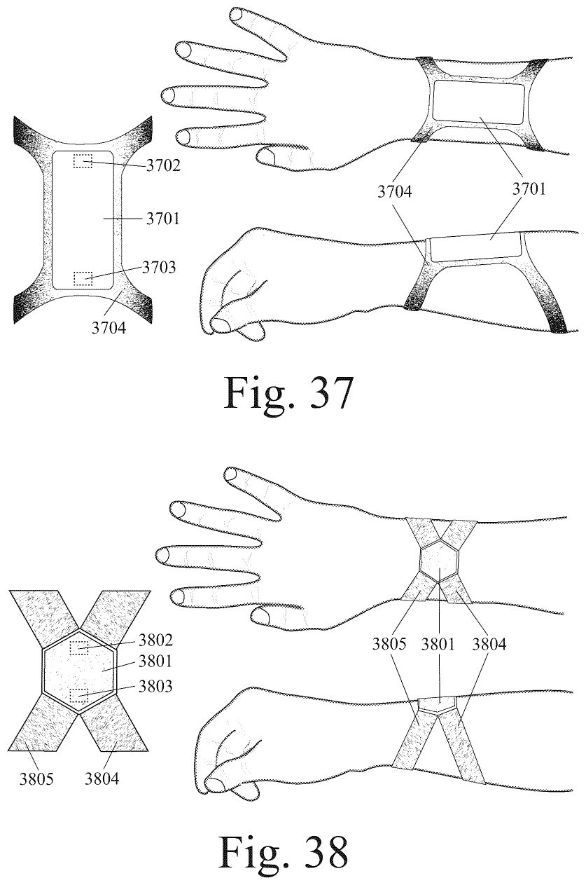

FIG. 37 shows an example of a wearable computing device for the wrist and/or arm comprising: rounded-rectangle display member 3701; data control unit 3702; sensor 3703; and longitudinal flared-two-strap attachment member 3704. The left portion of FIG. 37 shows a detailed top-down view of the device by itself. The upper-right portion of FIG. 37 shows a top-down view of this device on a person's forearm and the lower-right portion of FIG. 37 shows a side view of this device on a person's forearm. In an example, sensor 3703 and/or other sensors which are part of the device can be electromyography (EMG) sensors. In an example, the configuration and/or movement of a person's fingers, hand, and/or arm causes electromagnetic signals from the person's muscles which are detected by the EMG sensors.

In an example, a wearable computing device for the wrist and/or arm can comprise: a rounded-rectangular display member; a data control unit; electromyography (EMG) sensors; and a longitudinal flared two-strap attachment member. In an example, electromyography (EMG) sensors can be on the longitudinal flared two-strap attachment member. In an example, electromyography (EMG) sensors can be around the circumference of the longitudinal flared two-strap attachment member. In an example, electromyography (EMG) sensors can be on the ventral portion of the longitudinal flared two-strap attachment member.

In an example, a longitudinal flared-two-strap attachment member can hold a rounded-rectangle display member, a data control unit, and one or more sensors within three inches of the surface of a person's body. In an example, a longitudinal flared-two-strap attachment member can further comprise two straps or bands which each span the circumference of the person's wrist and/or arm. This holds the device onto the person's arm at two different locations along the longitudinal axis of the person's forearm. In an example, these straps or bands can be flexible. In an example, these straps or bands can each have portions which interconnect (such as with clips, clasps, snaps, buckles, or hook-and-eye) to fasten the device to the person's wrist and/or arm at two different places. In an example, these straps or bands can be stretched or expanded so that they can slip over a person's hand onto their wrist. Relevant variations discussed elsewhere in this disclosure or priority-linked disclosures can also be applied to this example.

FIG. 38 shows an example of a wearable computing device for the wrist and/or arm comprising: hexagonal display member 3801; data control unit 3802; sensor 3803; and straps 3804 and 3805. The left portion of FIG. 38 shows a detailed top-down view of the device by itself. The upper-right portion of FIG. 38 shows a top-down view of this device on a person's wrist. The lower-right portion of FIG. 38 shows a side view of this device on a person's wrist. A person's wrist or hand is considered to be part of their arm. In an example, sensor 3803 and/or other sensors which are part of the device can be electromyography (EMG) sensors. In an example, the configuration and/or movement of a person's fingers, hand, and/or arm causes electromagnetic signals from the person's muscles which are detected by the EMG sensors.

In an example, a wearable computing device for the wrist and/or arm can comprise: a hexagonal display member; a data control unit; electromyography (EMG) sensors; and a two straps. In an example, electromyography (EMG) sensors can be on the two straps. In an example, electromyography (EMG) sensors can be around the circumferences of the two straps. In an example, electromyography (EMG) sensors can be on the ventral portions of the two straps.

In an example, straps can be flexible straps or bands. In an example, portions of a strap can connect with each other (such as with a clip, buckle, snap, or hook-and-eye mechanism) to fasten it around a person's wrist and/or arm. In an example, straps can be sufficiently resilient or rigid that they fasten securely around a person's wrist and/or arm even though they span less than 100% of the circumference of the person's wrist and/or arm. In an example, straps can be made with flexible metal or a resilient polymer. In an example, straps can be stretchable, elastic, or expandable. In an example, straps can be made with a stretchable or elastic material, such as stretchable or elastic fabric. In an example, straps can comprise a series or chain of expandable, interconnected links. In an example, straps can be stretched or expanded to slip over a person's hand onto a person's wrist. Relevant variations discussed elsewhere in this disclosure or priority-linked disclosures can also be applied to this example.

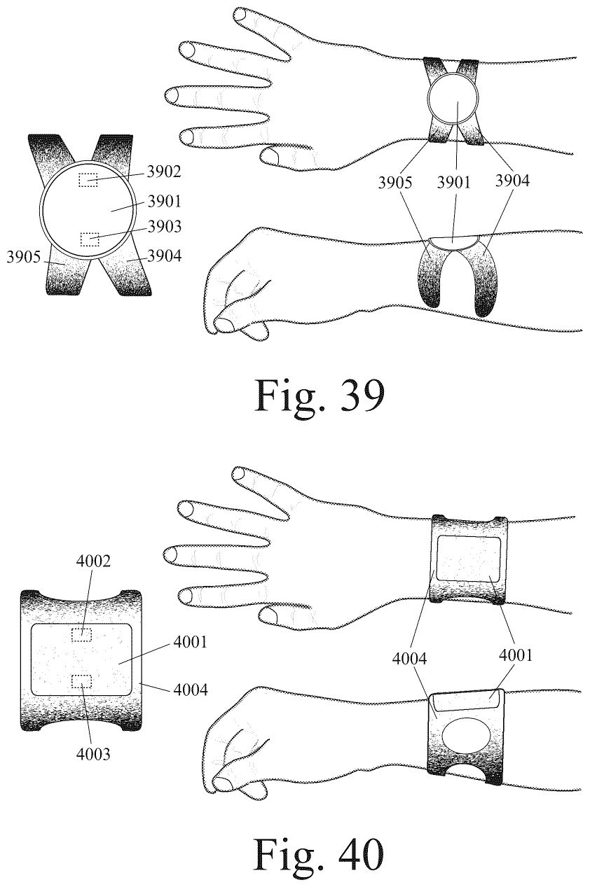

FIG. 39 shows an example of a wearable computing device for the wrist and/or arm comprising: arcuate display member 3901; data control unit 3902; sensor 3903; and flared circumferential attachment members 3904 and 3905. Within FIG. 39, the left portion shows a detailed top-down view of the device by itself, the upper-right portion shows a top-down view of this device on a person's wrist, and the lower-right portion shows a side view of this device on a person's wrist. A person's wrist, hand, finger, forearm, and upper arm are considered to be parts of their arm. In an example, sensor 3903 and/or other sensors which are part of the device can be electromyography (EMG) sensors. In an example, the configuration and/or movement of a person's fingers, hand, and/or arm causes electromagnetic signals from the person's muscles which are detected by the EMG sensors.

In an example, a wearable computing device for the wrist and/or arm can comprise: an arcuate display member; a data control unit; electromyography (EMG) sensors; and flared circumferential attachment members. In an example, electromyography (EMG) sensors can be on the flared circumferential attachment members. In an example, electromyography (EMG) sensors can be around the circumferences of the flared circumferential attachment members. In an example, electromyography (EMG) sensors can be on the ventral portions of the flared circumferential attachment members.

In an example, flared circumferential attachment members can each span between 50% and 95% of the circumference of a person's wrist and/or arm. In an example, flared circumferential attachment members can be sufficiently flexible that they can be flexed to fit around a person's arm, but are also sufficiently resilient to hold the device securely on the arm once they are fitted around the arm. In an example, flared circumferential attachment members can be made with flexible metal or a resilient polymer. In an example, flared circumferential attachment members can each span the entire circumference of the person's wrist and/or arm. In an example, portions of a flared circumferential attachment member can connect with each other (such as with a clip, buckle, snap, or hook-and-eye mechanism) to fasten it around a person's wrist and/or arm. In an example, flared circumferential attachment members can be stretched or expanded in order to slip over a person's hand onto a person's wrist. Relevant variations discussed elsewhere in this disclosure or priority-linked disclosures can also be applied to this example.

FIG. 40 shows an example of a wearable computing device for the wrist and/or arm comprising: arcuate display member 4001; data control unit 4002; sensor 4003; and holey attachment member 4004. Within FIG. 40, the left portion shows a top-down view of the device alone, the upper-right portion shows a top-down view of the device on a forearm, and the lower-right portion shows a lateral view of the device on a forearm. In an example, sensor 4003 and/or other sensors which are part of the device can be electromyography (EMG) sensors. In an example, the configuration and/or movement of a person's fingers, hand, and/or arm causes electromagnetic signals from the person's muscles which are detected by the EMG sensors.

In an example, a wearable computing device for the wrist and/or arm can comprise: an arcuate display member; a data control unit; electromyography (EMG) sensors; and a holey attachment member. In an example, electromyography (EMG) sensors can be on the holey attachment member. In an example, electromyography (EMG) sensors can be around the circumferences of the holey attachment member. In an example, electromyography (EMG) sensors can be on the ventral portions of the holey attachment member.

In an example, a holey attachment member can further comprise at least one hole which spans at least 50% of the width of the holey attachment member, wherein this width is a distance along the longitudinal axis of the forearm. In this example, a holey attachment member can hold an arcuate display member, a data control unit, and sensors within three inches of the surface of a person's body. In various examples, a holey attachment member: (a) can have buckles, snaps, adhesive, hook-and-eye mechanisms or other connecting elements so as to be fastened around the circumference of the forearm; (b) can be stretched or expanded around the hand to slip onto the forearm; (c) or can span between 50% and 95% of the circumference of the forearm and be flexible enough to bend around the forearm. Relevant variations discussed elsewhere in this disclosure or priority-linked disclosures can also be applied to this example.

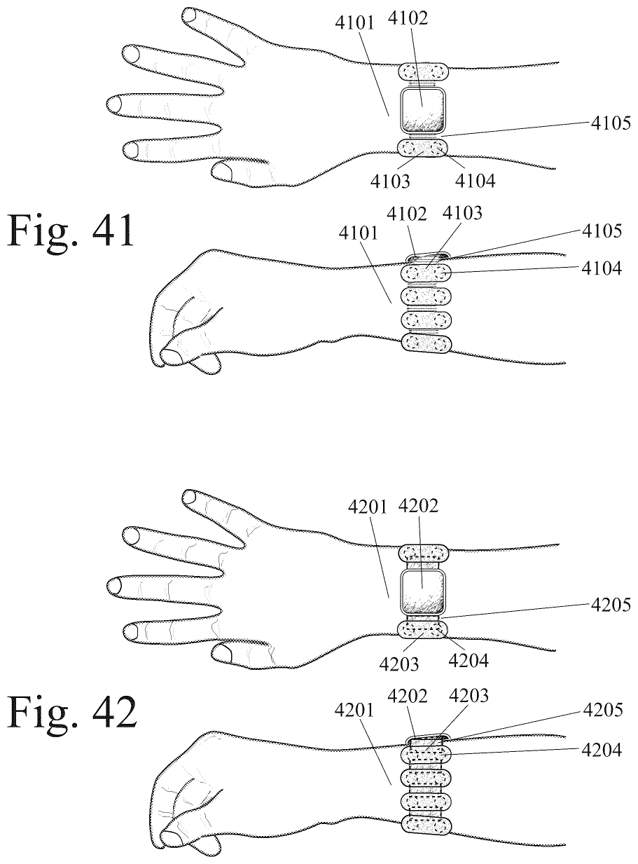

FIG. 41 shows two views of a wearable device (e.g. a band, smart watch, or watch band) with neuromuscular activity (e.g. EMG) sensors comprising: an annular (or partially-annular) sequence of rigid housings (including 4103) and stretchable (e.g. elastic) sections (including 4105) which is configured to be worn around at least 50% of a circumference of a person's wrist (and/or forearm) 4101, wherein the sequence alternates between rigid housings and stretchable sections around the person's wrist (and/or forearm); a plurality of neuromuscular activity (e.g. EMG) sensors (including sensor 4104) on the rigid housings; and a display screen (and/or watch housing) 4102 which is held on the wrist (and/or forearm) by the annular sequence. In an example, the device can further comprise a battery, an amplifier, a data processor, and/or a data transceiver. The upper portion of FIG. 41 shows a dorsal (e.g. top-down) view of the device and the lower portion of FIG. 41 shows a lateral (e.g. side) view of the device.

In an example, rigid housings can be made from metal. In an example, rigid housings can be made from a rigid polymer. In an example, stretchable sections can be made from fabric. In an example, stretchable sections can be made from elastic and/or air-permeable fabric. In an example, stretchable sections can further comprise flexible electroconductive pathways (e.g. sinusoidal wires or electroconductive threads) which transmit power to rigid housings and/or transmit data from the sensors. In an example, rigid housings can be wider than stretchable sections. In an example, stretchable sections can be piezoelectric. In an example, stretchable sections can be made with piezoelectric material, wherein stretchability and/or lengths of stretchable sections can be adjusted by transmission of electrical energy.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

FIG. 42 shows two views of a wearable device (e.g. a band, smart watch, or watch band) with neuromuscular activity (e.g. EMG) sensors comprising: an annular (or partially-annular) sequence of rigid housings (including 4203) and telescoping sections (including 4205) which is configured to be worn around at least 50% of a circumference of a person's wrist (and/or forearm) 4201, wherein the sequence alternates between rigid housings and telescoping sections around the person's wrist (and/or forearm); a plurality of neuromuscular activity (e.g. EMG) sensors (including sensor 4204) on the rigid housings; and a display screen (and/or watch housing) 4202 which is held on the wrist (and/or forearm) by the annular sequence. In an example, the device can further comprise a battery, an amplifier, a data processor, and/or a data transceiver. The upper portion of FIG. 42 shows a dorsal (e.g. top-down) view of the device and the lower portion of FIG. 42 shows a lateral (e.g. side) view of the device.

The adjective “telescoping” is colloquial generalization of the way in which the length of an old-fashioned telescope (or “spy glass”) is changed by changing the extent to which multiple cylindrical segments of the telescope overlapped. When the multiple cylindrical segments overlap more (e.g. are collapsed onto each other), then the overall length of the telescope decreases. When the multiple cylindrical segments overlap less (e.g. are extended out from each other), then the overall length of the telescope increases.

More generally, a telescoping section can comprise at least two components, wherein a first of the two components can be removably-inserted into a second of the two components. When the first component is inserted into the second component to a greater extent, then the overall length of the telescoping section is decreased. When the second component is inserted into the second component to a lesser extent, then the overall length of the telescoping section is increased. Although the components of old-fashioned telescopes (e.g. “spy glasses”) were cylindrical, the telescoping concept can be more generally applied to telescoping components with different-shaped perimeters (e.g. arcuate, polygonal, etc.).

In an example, components of telescoping sections in this device can connected by springs, pistons, or other tensile components so that they are held in tension and force is required to change the extent to which one component is inserted into another component. For example, components can be held in tension with each other so that the annular sequence can be (temporarily) expanded by the application of (temporary) force. For example, the diameter of the annular sequence can be temporarily increased by pulling so that it can be slipped around a person's hand to be placed on the person's wrist (and/or forearm). In an example, the amount of tension in springs, pistons, or other tensile components can be adjusted in order to change the amount of force required to expand the annular sequence. In an example, the amount of tension in springs, pistons, or other tensile components can be adjusted in order to change the fit (e.g. tighter or looser) of the annular sequence on a person's wrist (and/or forearm).

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

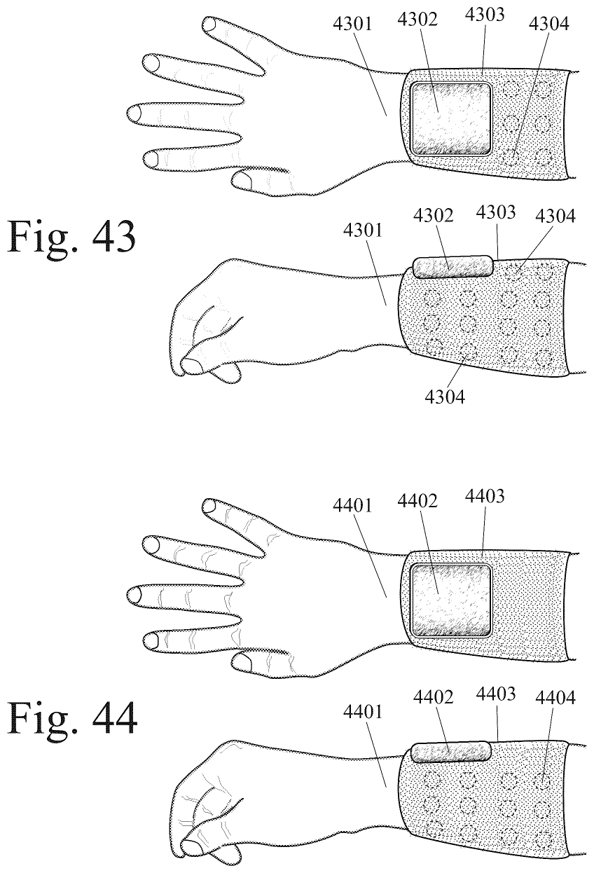

FIG. 43 shows two views of a wearable device (e.g. a band, smart watch, or watch band) with neuromuscular activity (e.g. EMG) sensors comprising: a stretchable fabric sleeve (or cuff) 4303 which is configured to be worn around a person's wrist (and/or forearm) 4301; a plurality of neuromuscular activity (e.g. EMG) sensors (including sensor 4304) on the sleeve, wherein there is at least one sensor on a portion of the sleeve which spans the dorsal surface of the person's wrist (and/or forearm); and a display screen (and/or watch housing) 4302 which is held on the wrist (and/or forearm) by the sleeve. In an example, the device can further comprise a battery, an amplifier, a data processor, and/or a data transceiver. The upper portion of FIG. 43 shows a dorsal (e.g. top-down) view of the device and the lower portion of FIG. 43 shows a lateral (e.g. side) view of the device.

In an example, neuromuscular activity (EMG) sensors can be distributed around the entire circumference of a person's wrist (and/or forearm). In an example, there can be neuromuscular activity (EMG) sensors on a portion of the sleeve which spans the ventral surface of the person's wrist (and/or forearm), a portion of the sleeve which spans the dorsal surface of the person's wrist (and/or forearm), and the portions of the sleeve which span the lateral (between ventral and dorsal) surfaces of the person's wrist (and/or forearm). In an example, neuromuscular activity (EMG) sensors can be distributed around a person's wrist (and/or forearm) in at least four circumferential rings. In an example, neuromuscular activity (EMG) sensors can be distributed around a person's wrist (and/or forearm) in at least one circumference ring which intersects the display screen (and/or watch housing) and in also at least one circumferential ring which is at least one inch proximal to the proximal edge of the display screen (and/or watch housing)

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes.

In an example, neuromuscular activity (EMG) sensors can be formed by weaving, sewing, braiding, or embroidering electroconductive yarns, threads, or fibers into the fabric used to create the sleeve. In an example, neuromuscular activity (EMG) sensors can be formed by selectively weaving, sewing, braiding, or embroidering electroconductive yarns, threads, or fibers into selected areas of the fabric used to create the sleeve before the sleeve is formed. In an example, neuromuscular activity (EMG) sensors can be formed by sewing, braiding, or embroidering electroconductive yarns, threads, or fibers onto the sleeve after the sleeve is formed. In an example, neuromuscular activity (EMG) sensors can be formed by printing electroconductive ink onto the sleeve. In an example, neuromuscular activity (EMG) sensors can be formed by adhering electroconductive strips or patches onto the sleeve. In an example, the sleeve can be made with elastic material. In an example, the sleeve can be made with elastic yarns, threads, or fibers.

In an example, a display screen (and/or watch housing) can be an integral part of a device. In an example, a device including the sleeve, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch sleeve which can hold a separate device (e.g. separate smart watch housing) on a person's wrist (or forearm). In an example, a watch sleeve with neuromuscular activity (e.g. EMG) sensors can be used to hold different types and/or brands of watch housings on a person's wrist (and/or forearm). In an example, a watch housing can be removably attached to a sleeve. Relevant variations discussed elsewhere in this disclosure or priority-linked disclosures can also be applied to this example.

FIG. 44 shows two views of a wearable device (e.g. a band, smart watch, or watch band) with neuromuscular activity (e.g. EMG) sensors comprising: a stretchable fabric sleeve (or cuff) 4403 which is configured to be worn around a person's forearm 4401; a plurality of neuromuscular activity (e.g. EMG) sensors (including sensor 4404) on the portions of the sleeve which span dorsal and lateral surfaces of the person's forearm; and a display screen (and/or watch housing) 4402 which is held on the forearm by the sleeve. In an example, the device can further comprise a battery, an amplifier, a data processor, and/or a data transceiver. The upper portion of FIG. 44 shows a dorsal (e.g. top-down) view of the device and the lower portion of FIG. 44 shows a lateral (e.g. side) view of the device.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes.

In an example, neuromuscular activity (EMG) sensors can be formed by weaving, sewing, braiding, or embroidering electroconductive yarns, threads, or fibers into the fabric used to create the sleeve. In an example, neuromuscular activity (EMG) sensors can be formed by selectively weaving, sewing, braiding, or embroidering electroconductive yarns, threads, or fibers into selected areas of the fabric used to create the sleeve before the sleeve is formed. In an example, neuromuscular activity (EMG) sensors can be formed by sewing, braiding, or embroidering electroconductive yarns, threads, or fibers onto the sleeve after the sleeve is formed. In an example, neuromuscular activity (EMG) sensors can be formed by printing electroconductive ink onto the sleeve. In an example, neuromuscular activity (EMG) sensors can be formed by adhering electroconductive strips or patches onto the sleeve. In an example, the sleeve can be made with elastic material. In an example, the sleeve can be made with elastic yarns, threads, or fibers.

In an example, a display screen (and/or watch housing) can be an integral part of a device. In an example, a device including the sleeve, sensors, and display screen can collectively comprise a smart watch. In an example, a device can comprise a forearm-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch sleeve which can hold a separate device (e.g. separate smart watch housing) on a person's forearm (or forearm). In an example, a watch sleeve with neuromuscular activity (e.g. EMG) sensors can be used to hold different types and/or brands of watch housings on a person's forearm. In an example, a watch housing can be removably attached to a sleeve. Relevant variations discussed elsewhere in this disclosure or priority-linked disclosures can also be applied to this example.

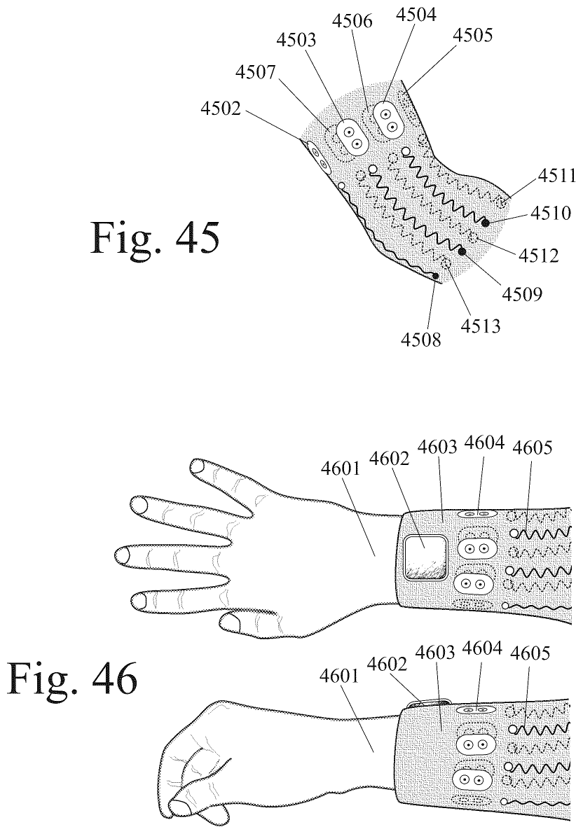

FIG. 45 shows a wearable device (e.g. a band, cuff, or sleeve) for measuring body motion and/or muscle activity comprising: a plurality of EMG sensors (4502, 4503, 4504, 4505, 4506, and 4507) which are distributed around a circumference of a person's arm (or wrist), wherein the EMG sensors collect electromagnetic energy data concerning neuromuscular activity of a set of muscles; and a plurality of bending-based (e.g. strain-based) motion sensors (4508, 4509, 4510, 4511, 4512, and 4513) distributed around the circumference of the person's arm (or wrist). In an example, the wearable device can further comprise a data processing unit which jointly analyzes both electromagnetic energy data from the EMG sensors and motion data from the motion sensors in order to measure and/or model body motion and/or muscle activity. In an example, data from EMG sensors and motion sensors can be jointly analyzed using an Artificial Neural Network (ANN) or other form of machine learning. Relevant variations discussed elsewhere in this disclosure or priority-linked disclosures can also be applied to this example.

FIG. 46 shows two views of a wearable device (e.g. a band, smart watch, or watch band) with neuromuscular activity (e.g. EMG) sensors comprising: a stretchable fabric sleeve (or cuff) 4603 which is configured to be worn around a person's forearm 4601; a plurality of EMG sensors (including 4604) which are distributed around a circumference of the person's forearm, wherein the EMG sensors collect electromagnetic energy data concerning neuromuscular activity of a set of muscles; a plurality of bending-based (e.g. strain-based) motion sensors (including 4605) distributed around the circumference of the person's forearm; and a display screen (and/or watch housing) 4602 which is held on the forearm by the sleeve (or cuff). In an example, the device can further comprise a battery, an amplifier, a data processor, and/or a data transceiver. The upper portion of FIG. 46 shows a dorsal (e.g. top-down) view of the device and the lower portion of FIG. 46 shows a lateral (e.g. side) view of the device. In an example, data from EMG sensors and motion sensors can be jointly analyzed using an Artificial Neural Network (ANN) or other form of machine learning. Relevant variations discussed elsewhere in this disclosure or priority-linked disclosures can also be applied to this example.

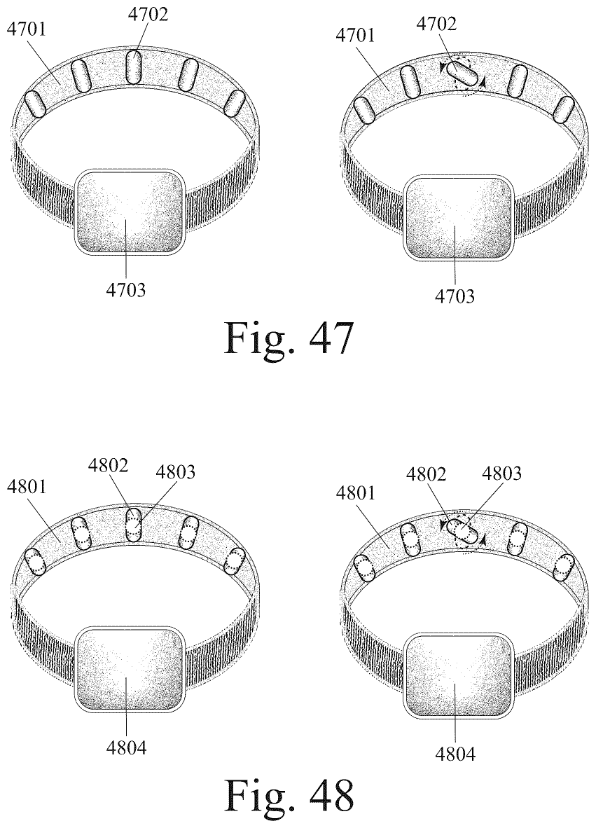

FIG. 47 shows two oblique views, at two different times, of a wearable device (e.g. a band, smart watch, or watch band) with selectively-rotatable neuromuscular activity (e.g. EMG) sensors. FIG. 47 shows the wearable device (e.g. a band, smart watch, or watch band) with selectively-rotatable neuromuscular activity (e.g. EMG) sensors comprising: a band (or strap) 4701 which is configured to be worn around at least 50% of a circumference of a person's wrist (and/or forearm); a plurality of selectively-rotatable neuromuscular activity (e.g. EMG) sensors (including sensor 4702) on the band; and a display screen (and/or watch housing) 4703 which is held on the wrist (and/or forearm) by the band. In an example, the device can further comprise a battery, an amplifier, a data processor, and/or a data transceiver. The left portion of FIG. 47 shows the device before the selected sensor has been rotated. The right portion of FIG. 48 shows the device before after the selected sensor has been rotated.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have longitudinal (e.g. oblong or rectangular) cross-sectional shapes. In an example, a sensor can have a first configuration in which its longitudinal axis is orthogonal to a circumference of a band and a second configuration in which its longitudinal axis intersects the circumference at an acute angle. In an example, one or more selected sensor can be changed from their first configurations to their second configurations by one or more electromagnetic actuators.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

FIG. 48 shows a wearable device (e.g. a band, smart watch, or watch band) with neuromuscular activity (e.g. EMG) sensors which is like the one shown in FIG. 47, except that it is further specified that the sensors rotate around their centers.

FIG. 48 shows two oblique views, at two different times, of a wearable device (e.g. a band, smart watch, or watch band) with selectively-rotatable neuromuscular activity (e.g. EMG) sensors. FIG. 48 shows the wearable device (e.g. a band, smart watch, or watch band) with selectively-rotatable neuromuscular activity (e.g. EMG) sensors comprising: a band (or strap) 4801 which is configured to be worn around at least 50% of a circumference of a person's wrist (and/or forearm); a plurality of selectively-rotatable neuromuscular activity (e.g. EMG) sensors (including sensor 4802) on the band, wherein a sensor rotates around its center (such as 4803); and a display screen (and/or watch housing) 4804 which is held on the wrist (and/or forearm) by the band. In an example, the device can further comprise a battery, an amplifier, a data processor, and/or a data transceiver. The left portion of FIG. 48 shows the device before the selected sensor has been rotated. The right portion of FIG. 48 shows the device before after the selected sensor has been rotated. Relevant variations discussed elsewhere in this disclosure or priority-linked disclosures can also be applied to this example.

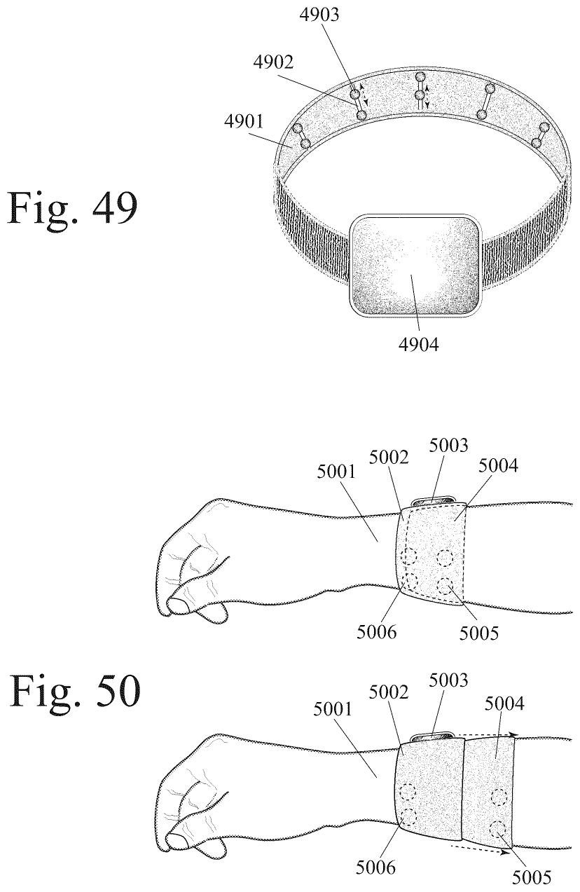

FIG. 49 shows an oblique view of a wearable device (e.g. a band, smart watch, or watch band) with neuromuscular activity (e.g. EMG) sensors comprising: a band (or strap) 4901 which is configured to be worn around at least 50% of a circumference of a person's wrist (and/or forearm); a plurality of lateral channels or tracks (including 4902) which span portions of the band laterally; a plurality of neuromuscular activity (EMG) sensors (including 4903), wherein one or more of the sensors can be selectively-moved along the lateral channels or tracks; and a display screen (and/or watch housing) 4904 which is held on the wrist (and/or forearm) by the band. In an example, the device can further comprise a battery, an amplifier, a data processor, and/or a data transceiver.

In an example, a lateral channel or track can span a portion of a width of the band. In an example, a lateral channel or track can span an entire width of the band. In an example, a lateral channel or track can span between 50% and 80% of a width of the band. In an example, a lateral channel or track can be orthogonal to a circumference of the band. In an example, a sensor can slide along a lateral channel or track. In an example, there can be one sensor on each lateral channel or track. In an example, there can be two or more sensors on the same lateral channel or track. In an example, a sensor can be manually slid along a lateral channel or track. In an example, a sensor can be automatically slid along a lateral channel or track by an electromagnetic actuator.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes.

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

FIG. 50 shows two lateral (side) views, at two different times, of a wearable device (e.g. a band, smart watch, or watch band) with neuromuscular activity (e.g. EMG) sensors comprising: a proximal band 5004 which is configured to be worn around at least 50% of a circumference of a person's wrist (and/or forearm) 5001; a distal band 5002 which is configured to be worn around at least 50% of a circumference of the person's wrist (and/or forearm) 5001, wherein the proximal band and the distal band have a first (retracted) configuration in which the proximal band and the distal band overlap by a first amount, wherein the proximal band and the distal band have a second (extended) configuration in which the proximal band and the distal band overlap by a second amount, and wherein the second amount is less than the first amount; a first plurality of neuromuscular activity (e.g. EMG) sensors (including sensor 5005) on the proximal band; a second plurality of neuromuscular activity (e.g. EMG) sensors (including sensor 5006) on the distal band; and a display screen (and/or watch housing) 5003 on the distal band. In an example, the device can further comprise a battery, an amplifier, a data processor, and/or a data transceiver. The upper portion of FIG. 50 shows this device in the first (retracted) configuration. The upper portion of FIG. 50 shows this device in the second (extended) configuration.

In this example, the proximal band is closer to the person's shoulder and the distal band is farther from the person's shoulder. In an example, the proximal band can overlap the distal band to a first extent in the first configuration and the proximal band can overlap the distal band to a second extent in the second configuration, wherein the second extent is less than the first extent. In an example, the proximal band can be inserted into, under, or over the distal band to a first extent in the first configuration and the proximal band can be inserted into, under, or over the distal band to a second extent in the second configuration, wherein the second extent is less than the first extent.

In an example, the proximal band and the distal band can together span a first distance on the person's wrist (and/or forearm) in the first configuration and a second distance on the person's wrist (and/or forearm) in the second configuration, wherein the second distance is greater than the first distance. In an example, the proximal and distal bands can together comprise a multi-section telescoping band (or sleeve). In an example, distances between sensors and/or electrode pairs in sensors can be selectively changed (e.g. adjusted) by changing the extent to which the proximal and distal band overlap.

In an example, neuromuscular activity sensors can be electromyography (EMG) sensors. In an example, neuromuscular activity sensors can comprise electrodes. In an example, neuromuscular activity sensors can comprise pairs of electrodes. In an example, neuromuscular activity sensors can be capacitive EMG sensors. In an example, neuromuscular activity sensors can be resistive EMG sensors. In an example, neuromuscular activity sensors can be surface EMG sensors. In an example, neuromuscular activity sensors can have circular cross-sectional shapes. In an example, neuromuscular activity sensors can have longitudinal (e.g. oblong or rectangular) cross-sectional shapes, wherein their longitudinal axes are orthogonal to the circumference of a band and/or a person's wrist (or forearm).

In an example, a display screen (and/or watch housing) can be an integral part of a device. In an example, a device including the band, sensors, and display screen can collectively comprise a smart watch. In an example, a device can comprise a wrist-worn computing device and/or motion recognition device with a display screen which is more generic than that which is currently categorized as a smart watch. In an example, a watch housing, a battery, and/or other components in a such a housing can be in electromagnetic communication and/or power transmission connection with neuromuscular activity (e.g. EMG) sensors.

In an example, a display screen (and/or watch housing) can be separate from a device. In an example, a device can comprise a watch band (or strap) which can hold a separate device (e.g. separate smart watch housing) on a person's wrist (or forearm). In an example, a watch band (or strap) with neuromuscular activity (e.g. EMG) sensors can be used to hold different types and/or brands of watch housings on a person's wrist (and/or forearm). In an example, a watch housing can be removably attached to a band (or strap). In an example, a band which functions as a watch band (or strap) can span between 50% and 90% of the circumference of a person's wrist (and/or forearm). In variations on this example, a strap, bracelet, cuff, and/or sleeve can function as a band in a device. Relevant variations discussed elsewhere in this disclosure or priority-linked disclosures can also be applied to this example.

I claim:

1. A wearable device with neuromuscular activity sensors comprising:
a band which is configured to be worn around at least 50% of a circumference of a person's wrist or forearm, wherein the average width of a portion of the band on the ventral surface of the person's wrist or forearm is at least 20% greater than the average width of a portion of the band on the dorsal surface of the person's wrist or forearm; and
a plurality of neuromuscular activity sensors on the band.

2. The device in claim 1 wherein the device further comprises a display screen or watch housing.

3. The device in claim 2 wherein the width of the band tapers toward the display screen or watch housing.

4. The device in claim 1 wherein the band bifurcates into proximal and distal branches.

5. A wearable device with neuromuscular activity sensors comprising:
a band which is configured to be worn around at least 50% of a circumference of a person's wrist or forearm;
a length-adjusting mechanism on an end of the band; and
a plurality of neuromuscular activity sensors on the band.

6. The device in claim 5 wherein the device further comprises a display screen or watch housing.

7. The device in claim 6 wherein there is a length-adjusting mechanism on each end of the band where the band is connected to the display screen or watch housing.

8. The device in claim 5 wherein a length-adjusting mechanism comprises a roller or axle around which an end of the band loops to overlap a portion of the rest of the band.

9. The device in claim 5 wherein a length-adjusting mechanism is an elastic and/or stretchable section.

10. The device in claim 5 wherein a length-adjusting mechanism is a telescoping section.

11. The device in claim 5 wherein a length-adjusting mechanism is a housing in which an end of the band is coiled.

12. A wearable device with neuromuscular activity sensors comprising:
a band which is configured to be worn around at least 50% of a circumference of a person's wrist or forearm;
a channel or track on the band; and
a plurality of neuromuscular activity sensors on the band, wherein one or more of the neuromuscular activity sensors can be selectively moved along the channel or track.

13. The device in claim 12 wherein the device further comprises a display screen or watch housing.

14. The device in claim 12 wherein the channel or track is around a portion of the circumference of the band.

15. The device in claim 12 wherein the channel, track, or ring is centered within a width of the band.

16. The device in claim 12 wherein the channel or track is across a portion of the width of the band.

17. The device in claim 12 wherein the sensors are manually moved along the channel or track.

18. The device in claim 12 wherein the sensors are automatically moved along the channel or track by an actuator.

19. The device in claim 12 wherein the sensors are moved to more accurately record the activity of specific muscles.

20. The device in claim 12 wherein the sensors are moved to customize the sensor configuration to the anatomy of a specific person.
